(19) United States
(12) Patent Application Publication
(10) Pub. No.: US 2023/0192845 A1
(43) Pub. Date: Jun. 22, 2023

(54) CD96-BINDING AGENTS AS IMMUNOMODULATORS

(71) Applicants: BLINK BIOMEDICAL SAS, Marseille (FR); TALIX THERAPEUTICS NV, Leuven (BE)

(72) Inventors: Florence Renart-Depontieu, Saint-Priest (FR); Sachiko Takami, Toyama (JP); Germain Margall-Ducos, Marseille (FR); Nicola Beltraminel-Li, Marseille (FR); Pierre Garrone, Lyon (FR); Anne Rogel, Southampton Hampshire (GB); Aymen Al-Shamkhani, Southampton Hampshire (GB); Xavier Preville, St. Louis (FR)

(73) Assignees: BLINK BIOMEDICAL SAS, Marseille (FR); TALIX THERAPEUTICS NV, Leuven (BE)

(21) Appl. No.: 16/636,947

(22) PCT Filed: Aug. 10, 2018
(86) PCT No.: PCT/EP2018/071746

§ 371 Date: Feb. 6, 2020

(30) Foreign Application Priority Data
Aug. 11, 2017 (EP) 17185944.0

(51) Int. Cl. C07K 16/28; A61K 39/395; A61K 45/06; A61P 35/00

(52) U.S. Cl. CPC C07K 16/2803; A61K 39/3955; A61K 45/06; A61P 35/00; C07K 2317/74; C07K 2317/34; C07K 2317/565; C07K 2317/75; C07K 2317/92; C07K 2317/33; C07K 2317/24

(57) ABSTRACT

Provided is a CD96-binding agent, wherein the agent is capable of stimulating activation and/or proliferation of T cells upon binding to CD96. Also provided are isolated nucleic acids and cells that produce the agent and uses thereof.

### SEQUENCE LISTING

This application incorporates by reference the material in the ASCII text file “2020-05-17 Substitute Sequence Listing BBX0001PA.txt” of 109,065 bytes created on May 17, 2020, and filed herewith.

### FIELD OF THE INVENTION

The invention relates to agents that modulate (increase or inhibit) immune cell function. In one aspect, the agents bind to CD96 and stimulate activation and/or proliferation of T cells. The agents can find use in various therapeutic methods, in particular in the treatment of cancer or infectious diseases. In one aspect, the agents are antibodies or fragments thereof that activate signaling via CD96, e.g. anti-CD96 agonist antibodies. The invention includes isolated antibodies and derivatives and fragments thereof, pharmaceutical formulations comprising one or more mouse or chimeric anti-hCD96 monoclonal antibodies; and cell lines that produce these recombinant monoclonal antibodies.

### BACKGROUND OF THE INVENTION

CD96 (TACTILE for T cell activation, increased late expression) belongs to immunoglobulin superfamily receptors located on the surface of NK and T cells. The Ig superfamily also includes CD226 (DNAM-1, for DNAX accessory molecule-1) (Shibuya, A., et al. DNAM-1, a novel adhesion molecule involved in the cytolytic function of T lymphocytes. Immunity 4, 573-581 (1996)), TIGIT (T cell immunoglobulin and ITIM domain) (Yu, X, et al. the surface protein TIGIT suppresses T cell activation by promoting the generation of mature immunoregulatory dendritic cells. Nature immunology 10, 48-57 (2009)) and CRTAM (Class I restricted T cell-associated molecules) (Kennedy, J., et al. A molecular analysis of NKT cells: identification of class-I restricted T cell-associated molecule (CRTAM). Journal of leukocyte biology 67, 725-734 (2000)).

Human CD96 has been originally found on T cells and described as not to be expressed by B cells, monocytes, granulocytes, platelets and red blood cells (Wang et al., Identification and molecular cloning of tactile. A novel human T cell activation antigen that is a member of the Ig gene superfamily. J. Immunology 148, 2600-2608 (1992)). Despite being cloned 20 years ago (Wang, 1992), the function of human CD96 immuno-receptor remains to be controversial.

CD96 shares CD155 ligand with DNAM-I and TIGIT (Seth S., et al. The murine pan T cell marker CD96 is an adhesion receptor for CD155 and nectin-1. Biochemical and biophysical research communications 364, 959-965 (2007)). It has also been reported that CD96 interacts with CD111 (nectin-1) in the mouse and plays a role in promoting NK and T cell adhesion (Fuchs et al. Cutting edge: CD96 (tactile) promotes NK cell-target cell adhesion by interacting with the poliovirus receptor (CD155). J Immunol 172, 3994-3998 (2004)). In mice, CD96 is described as a negative immune-regulator of NK cells which competes with CD226 for binding to CD155 (Chan et al. The receptors CD96 and CD226 oppose each other in the regulation of natural killer cell functions. Nature Immunology, (2014)). Conversely, in humans, CD96 is described either as a weak (co)activator of NK cells cytotoxicity (Fuchs, 2004, Stanietsky et al. The interaction of TIGIT with PVR and PVRL2 inhibits human NK cell cytotoxicity. Proc Natl Acad Sci USA. 106(42):17858-17863 (2009)) or as an inhibitor of NK cells which inhibits IFN-γ production upon interaction with CD155 (WO 2015024060, Blake et al., Molecular Pathways: Targeting CD96 and TIGIT for Cancer Immunotherapy, Clin Cancer Res. 22(21):5183-5188 (2016)). No data have been published on the role of CD96 on modulation (inhibition or activation) of mouse and human T cell activity. Therefore, since the biology of human CD96 is poorly described, several mechanisms of actions of this receptor have been evaluated leading to the identification that human CD96 is a co-activating receptor on human T cells.

In the mouse, CD96 is described as a negative immune-regulator mainly on NK cells and mainly by competing with CD226 binding for the CD155 interaction (Chan, 2014; Blake, 2016). Inhibition of CD96 decreases tumor growth, metastasis and protects mice in different cancer models (Chan, 2014; Blake, 2016). The described effect of CD96 is mainly on NK cells, IFN-γ production and is dependent on the function of CD226. CD226 has been described to be expressed on TCRαβ⁺ T cells, TCRγδ⁺ T cells, NK cells, monocytes and a subset of B cells but not on granulocytes and erythrocytes (Shibuya, 1996).

Conversely, in human, CD96 is described as an adhesion molecule and as a weak co-activator of NK cell cytotoxicity (Wang, 1992; Fuchs, 2004). The Smyth's group has shown that CD96 may also act as weak inhibitor of CD155-induced IFN-γ secretion by human NK cells (WO2015024060).

No data have been published on the role of CD96 on mouse and human T cells, except on a role of mCD96 in favoring T cell adhesion and transmigration through CD155-expressing cells and in transmigration (Seth, 2007). Therefore, the biology of human CD96 is controversial and poorly described.

Accordingly, there is a need for improved agents and methods targeting CD96 for use in the treatment of disease.

### SUMMARY OF THE INVENTION

The aim of the invention is to provide CD96-binding agents capable of modulating immune cell activation for therapeutic uses.

The present invention describes for the first time that human CD96 can function as a co-stimulatory receptor on T cells, and discloses the identification of the first CD96-binding agents (e.g. anti-CD96 antibodies) capable of stimulating T cell activity and/or proliferation. Thus, in one aspect the present invention provides agonistic anti-CD96 antibodies, e.g. antibodies capable of activating intracellular signaling in T cells upon CD96 engagement. A CD96 agonist according to the invention is an agonist that indirectly or directly stimulates cells, e.g. T-cells, and/or indirectly or directly induces proliferation of cells, e.g. T-cells.

In one aspect the present invention provides a CD96-binding agent, wherein the agent is capable of stimulating activation and/or proliferation of T cells upon binding to CD96.

In one embodiment, the CD96-binding agent is capable of stimulating activation and/or proliferation of T cells in combination with a T cell co-stimulatory agent. Preferably the CD96-binding agent is a CD96 agonist. In one embodiment, binding of the agent to CD96 induces dimerization or multimerization of CD96. Preferably the T cells are CD4+ or CD8+ cells. In another embodiment, the CD96-binding agent is capable of stimulating activation and/or proliferation of T cells in humans.

Preferably the agent binds to human CD96, more preferably human CD96 variant 1 (SEQ ID NO: 267) and/or human CD96 variant 2 (SEQ ID NO: 268) and/or to rhesus CD96 (SEQ ID NO: 272). In one embodiment, the agent specifically binds to at least one epitope in an extracellular domain D1 (SEQ ID NO: 269), domain D2 v1 or v2 isoform (SEQ ID NO: 270 or 273), domain D3 (SEQ ID NO: 271) or domain D4 (SEQ ID NO: 274) of human CD96. For instance, the agent may specifically bind to an epitope shared by domains D1 and D2, or domains D2 and D3 of human CD96.

In a preferred embodiment, the agent is capable of binding to human CD96 with a dissociation constant (KD) of less than 50 nM, preferably less than 10 nM, most preferably less than 1 nM. For instance, the agent may be capable of binding to human CD96 with a dissociation constant (KD) of 0.01 to 50 nM, preferably 0.1 to 10 nM, most preferably 0.1 to 1 nM.

In another embodiment, the CD96-binding agent at least partially inhibits binding of CD96 to CD155. For instance, the CD96-binding agent may inhibit the binding of human CD96 to human CD155 with an IC50 value of less than 50 nM, preferably less than 25 nM, most preferably less than 10 nM. More preferably the CD96-binding agent inhibits the binding of human CD96 to human CD155 with an IC50 value of 0.01 to 50 nM, preferably 0.1 to 25 nM, most preferably 1 to 10 nM.

In an alternative embodiment, the CD96-binding agent does not inhibit binding of CD96 to CD155, or the CD96-binding agent inhibits the binding of CD96 to CD155 only weakly. For instance, in one embodiment the CD96-binding agent has an IC50 value for inhibition of the binding of human CD96 to human CD155 of 50 nM or higher, preferably 100 nM or above, most preferably 200 nM or above.

In one aspect, the CD96-binding agent is selected from the list comprising an antibody, an antibody fragment, an antibody mimetic, an antibody mimetic fragment, a nanobody or a small molecule inhibitor.

In a particular aspect, the CD96-binding agent is an antibody or an antibody fragment thereof. In a further aspect, the antibody is a chimeric, humanized or fully human antibody. In another embodiment, the antibody is an antibody fragment, preferably wherein the antibody fragment is a F(ab′)2 or a Fab fragment. In another embodiment, the antibody is a monospecific antibody, a bispecific antibody or a multispecific antibody.

In another aspect, the CD96-binding agent is an antibody mimetic or an antibody mimetic fragment thereof. In particular, said antibody mimetic is selected from the group comprising affibody molecules, affilins, affimers, affitins, alphabodies, anticalins, avimers, DARPins, fynomers, monobodies, aptamers, beta-hairpin mimetics, non-immunoglobulin scaffolds, or fusion proteins.

In still another aspect, the CD96-binding agent is a nanobody. In another further embodiment, the CD96-binding agent is an antibody-drug conjugate. In another further aspect, the CD96-binding agent is an antibody-scaffold fusion format, for example a Mabfilin or an Fabfilin.

In preferred embodiments, the antibody comprises a heavy chain having 3 CDR sequences set forth in SEQ ID NOs: 1-3, 13-15, 61-63, 73-75, 85-87, 109-111, 145-147, 157-159, 169-171, 181-183, 193-195, 205-207 or 217-219 (Kabat annotation) or SEQ ID NOs: 4-6, 16-18, 64-66, 76-78, 88-90, 112-114, 148-150, 160-162, 172-174, 184-186, 196-198, 208-210 or 220-222 (IMGT annotation) and/or a light chain having 3 CDR sequences set forth in SEQ ID NOs: 7-9, 19-21, 67-69, 79-81, 91-93, 115-117, 151-153, 163-165, 175-177, 187-189, 199-201, 211-213 or 223-225 (Kabat annotation) or SEQ ID NOs: 10-12, 22-24, 70-72, 82-84, 94-96, 118-120, 154-156, 166-168, 178-180, 190-192, 202-204, 214-216 or 226-228 (IMGT annotation). All SEQ IDs and corresponding sequences are listed in Table 5.

In another embodiment, the antibody comprises a heavy chain variable domain and/or a light chain variable domain comprising an amino acid sequence set forth in any one of SEQ ID NOs: 229-232, 239-244, 247-248, or 253-266.

In another aspect, the invention provides an isolated nucleic acid encoding the amino acid sequence of the CD96-binding agent as described above. Also provided is an isolated cell that produces the CD96-binding agent.

In another aspect, the invention provides a pharmaceutical composition comprising the CD96-binding agent as described above and a pharmaceutical acceptable excipient or carrier.

In another aspect, the invention provides a CD96-binding agent or pharmaceutical composition as described above for use for the treatment of a disease in a patient. Preferably the disease is a cancer or an infectious disease.

In one embodiment, the treatment is a monotherapy The agonist CD96-binding agent may be used alone to stimulate T cells to proliferate in vivo in a tumor micro-environment or in chronic infectious disease environment. Examples of agonist antibodies that can be used alone to stimulate T cells in a such a way, include but are not restricted to anti-CD28, anti-CD27, anti-CD137, anti-GITR, anti-OX40 and anti-ICOS (Sanmamed et al. Agonists of co-stimulation in cancer immunotherapy directed against CD137, OX40, GITR, CD27, CD28 and ICOS. Seminars in oncology. 42(4):640-655 (2015)).

In another embodiment the treatment is a combination therapy. For instance, the agent may be used in combination with a stimulating agent, a T-cell stimulating or proliferation agent, a T cell co-stimulating agent or with an immune checkpoint inhibitor.

In a further embodiment, the CD96-binding agent may be used in combination with a T-cell stimulating or proliferation agent. T-cell stimulating or proliferation agents include but are not restricted to: anti-CD3 monoclonal antibody, phorbol myristate acetate and ionomycin, super antigens such as staphyloccocal enterotoxins, toxic shock syndrome toxin 1, exfoliative toxin, streptococcal pyrogenic toxin or agglutinin such as wheat germ agglutinin or phyto-hemagglutinin.

In another embodiment, the CD96-binding agent may be used in combination with a T-cell co-stimulating agent. T-cell co-stimulating agents include but are not restricted to: anti-CD28, anti-ICOS, anti-CD226, anti-CD40L, anti-CD27, anti-HVEM, anti-OX40, anti CD137 or anti-GITR monoclonal antibodies.

In another embodiment, the CD96-binding agent may be used in combination with a stimulating agent. Stimulating agents include but are not restricted to: anti-CD40 or anti-HVEM monoclonal antibodies.

In still another embodiment, the CD96-binding agent may be used in combination with an immune checkpoint inhibitor. Immune checkpoint inhibitors include but are not restricted to: anti-CEACAM1, anti-TIM3, anti-CD80, anti-BTLA, anti-CD160, anti-VISTA, anti-PD1, anti-HVEM, anti-CTLA4, anti-PDL1 or anti-TIGIT monoclonal antibodies.

In another instance, the CD96-binding agent may be used in combination with a cancer cell or immunosuppressive cell killing agent. For example, the cancer cell or immunosuppressive cell killing agent may be radiotherapy or a chemotherapeutic agent. Chemotherapeutic agents include but are not restricted to: alkylating agents, nitrogen mustards, nitrosoureas, platinum agents, antimetabolites, natural products, anti-tumor antibiotics, anthracyclines, epipodophyllotoxins, vinca alkaloids, taxanes and camptothecin analogs. The cancer cell or immunosuppressive cell killing agent may be an antibody drug conjugate associated with any type of chemotherapeutic as listed above. In another embodiment, the cancer cell or immunosuppressive cell killing agent may be an inhibitor/antagonist/decoy of a signaling pathway. Targeted signaling pathways include but are not restricted to: EGFR and MAP kinase, PI3K, Akt, mTOR, ALK and ROS, cellular metabolism, autophagy, apoptosis, and angiogenesis. In a further embodiment, the cancer cell or immunosuppressive cell killing agent may be an inhibitor or antagonist of the DNA damage repair system. Pathways of DNA repair system include but are not restricted to: homologous recombination, mismatch repair, nucleotide excision repair, DNA strand crosslink repair and non-homologous end joining. In another embodiment, the CD96-binding agent may be used in combination with an agent to treat infectious diseases. Treatments for infectious diseases include but are not restricted to: antibiotics, antivirals, antifungals and anti-parasitics.

In another aspect, the invention provides the use of a CD96-binding agent or pharmaceutical composition according to the different embodiments of the invention for stimulation of the activation and/or proliferation of T cells upon binding to CD96. In a further embodiment, said use is characterized in that the CD96-binding agent is capable of proliferating T cells in combination with a T cell co-stimulatory agent and/or T cell proliferation agent. In still a further embodiment, the CD96-binding agent is a CD96 agonist. In still a further embodiment, said use is characterized in that binding of the agent to CD96 induces dimerization or multimerization of CD96. In another embodiment, said use is characterized in that the T cells are CD4⁺ and/or CD8⁺ T cells.

### BRIEF DESCRIPTION OF FIGURES

FIG. 1: Expression (A, B & C) and co-expression (D and E) of CD96 and CD226 on human PBMC subsets. The anti-CD96 (clone 628211) and anti-CD226 (clone DX11) antibodies were used to study the co-expression of CD96 and CD226 on CD56⁺ (NK & NKT cells), CD3⁺ (T and NKT cells), CD3⁺CD4⁺ T cells, CD3⁺CD8⁺ T cells, CD14⁺ (Monocytes) and CD19⁺ (B cells) by flow cytometry. A and C) % of cells expressing CD96 and CD226 in the different cell populations. B) MFI of CD96 in the different populations. D) % of cells co-expressing CD96 and CD226 for the indicated cell subsets. E) % of cells expressing CD96 among the CD226 positive cells for each of the indicated cell subsets. Data obtained from 4-13 independent samples from healthy donors.

FIG. 2: Expression of CD96 and CD226 on human naïve and memory CD4+ and CD8+ T cells. The expression of CD226 and CD96 was analyzed by multiple staining by FCM on CD3+CD4+ and CD3+CD8+ T cells gated from purified PBMCs (n=4). Memory T cells were further identified by the expression of CD45RO antigen. The median fluorescence intensity (MedFI) of CD96 and CD226 on the different T cell subsets is shown in the lower panels.

FIG. 3: Increased expression of CD96 and CD226 on anti-CD3-activated human CD4+ and CD8+ memory T cells. PBMC purified from healthy donors were activated with soluble anti-CD3 mAb OKT3. At different times indicated, the cells were collected and stained with anti-CD4, anti-CD8 and anti-CD45RO antibody, and with anti-CD96 or anti-CD226 antibodies. The mean fluorescence intensity (MFI) of CD96 and CD226 on CD45RO+ memory and CD45RO− naïve cells is measured by FCM on CD4+ and CD8+ T cells.

FIG. 4: Expression of hCD96 and hCD226 on human circulating CD4+ Tregs and conventional CD4+ T cells of healthy individuals. The expression of CD226 and CD96 was analyzed by flow cytometry on conventional and regulatory CD4+ T cells gated from purified PBMCs (n=4). Flow cytometry overlay histogram of CD226 (left) and CD96 (right) expression on conventional (thin dashed line: isotype control, thick plain line: anti-CD226 or anti-CD96) and regulatory T cells (filled gray: isotype control, thick dashed line: anti-CD226 or anti-CD96) are shown. The median of fluorescence of CD226 (left) or CD96 (right) on CD25hi CD12710 CD4+ regulatory T cells (open squares) and CD2510 CD127+CD4+ conventional T cells (open circles) are shown.

FIG. 5: Homo-dimerization and hetero-dimerization of hCD96 by FRET assay. CHO were transfected with (A) hCD226-His, (B) hCD226-His and hTIGIT-Myc, (C) hCD96-HA and (D) hCD96-HA and hCD226-His. After 24 hours the cells were labelled with (A) anti-hCD226-PE (clone DX11) and anti-hCD226-APC (clone DX11), (B) anti-hCD226-PE (clone DX11) and anti-hTIGIT-APC (clone 741182), (C) anti-HA-PE (clone GG8-1F3.3.1) and anti-HA-APC (clone GG8-1F3.3.1) and (D) anti-hCD226-PE (clone DX11) and anti-HA-APC (clone GG8-1F3.3.1), either separately then pooled (right panel, negative control of FRET) or simultaneously (left panel).

FIG. 6: Binding profiles of anti-hCD96 antibodies on human CD96-transfected CHO cells (hCD96v2, short isoform). CHO cells expressing hCD96v2 were incubated with serial dilution of the indicated mIgG1 or mIgG2a anti-CD96 or isotype control antibodies. After incubation at +4° C. and washings, binding of antibodies was revealed by addition of PE-conjugated anti-mIgG antibody and quantified by flow cytometry. The results are expressed as the difference of mean fluorescence intensity (delta MFI) between cells stained with antibodies and cells without antibodies. Dose response curves were obtained from GraphPad prism software using a non-linear regression fit with a variable slope model (4 parameters). To simplify the reading, results from the various anti-hCD96 candidates are shown on 4 different panels.

FIG. 7: Binding profiles of anti-CD96 antibodies on CHO cells transfected with rhesus CD96. Anti-CD96 antibodies or the corresponding isotype control mAbs were tested for their capacity to recognize rhesus CD96 transiently expressed on CHO cells by flow cytometry. Each histogram represents the overlay of the fluorescence intensity obtained for each antibody tested at 1 (black line) and 10 μg/ml (thick dashed black line) and compared to the fluorescence obtained with an isotypic control (filled grey histogram).

FIG. 8: Inhibition of hCD155 binding to hCD96 expressed on CHO cells by anti-CD96 antibodies (mean of 2 independent experiments). CHO cells expressing hCD96v2 were incubated with serial dilution of the indicated anti-CD96 or isotype control antibodies in the presence of biotinylated hCD155-Fc. After incubation at +4° C. and washings, the binding of hCD155 to CHO cells was revealed by addition of FITC-conjugated Streptavidin and quantified by flow cytometry. The results are expressed as the % of inhibition of CD155 binding to CD96. Dose response curves were obtained from GraphPad prism software using a non-linear regression fit with a variable slope model (4 parameters). To simplify the reading, results from the various anti human CD96 clones are shown on 4 different panels.

FIG. 9: (A) Binding of anti-hCD96 candidates and benchmarks on human T (left panel) and NK cells (right panel) isolated from healthy donors. The results show the difference of the mean fluorescence intensity (delta MFI) between the fluorescence measured with each anti-CD96 antibody and the MFI measured with the isotype control antibody (antibodies tested at 10 μg/mL). For each antibody, individual data points represent the values obtained with cells isolated from 3 healthy donors. (B) Expression of CD96 on tumor infiltrating lymphocytes extracted from head and neck tumors obtained from 4 patients (HN314, 315, 306 and 316). For each lymphocyte subset evaluated (CD8+ T cells, NK cells, Treg cells and conventional CD4+ T cells), overlay histograms show background staining by isotype control (gray shaded) and anti-CD96 mAb (thick black line).

FIG. 10: Mapping of the CD96 epitopes recognized by the different anti-CD96 candidates. Expression plasmids containing either the complete sequence of hCD96v2 or different extracellular domains (D1 to D3) of hCD96v2 or combinations thereof (D1D2, D1D3 or D2D3) were transiently transfected in CHO-S cells. Each transient transfect was indirectly stained with the different anti-CD96 candidates and analyzed by flow cytometry. Represented is the median % of stained CHO-S transfect obtained from 3 to 4 independent experiments for each anti-CD96 tested.

FIG. 11: Immobilized anti-CD96 candidate BL006-4-20 co-stimulates human CD4+ and CD8+ T cell activation with an anti-CD3 antibody. T cell activation was measured by the expression of CD25 on CD4+ and CD8+ T cells in PBMC isolated from a representative healthy donor (Donor #1) and cultured in the presence of soluble OKT3 at 0.1 or 1 ng/mL, with or without the anti-CD96 candidate BL006-4-20 or an isotype control mAb coated at 10 μg/mL. CD25 expression was measured by FCM after 3 days of culture on CD3+/CD4+ and CD3+/CD8+ gated T cells. Vertical dashed lines mark threshold for CD25 positivity on each set of histograms.

FIG. 12: Immobilized anti-CD96 candidates co-stimulate human CD4+ and CD8+ T cell activation with an anti-CD3 antibody. T cells activation is measured by the expression of CD25 on CD4+ and CD8+ T cells by FCM at day 3 after culture initiation of PBMC isolated from a representative healthy donor (Donor #1). PBMC were cultured with 0.1 or 1 ng/mL of soluble anti-CD3 OKT3 with (grey bars) or without (white bars) 10 μg/mL of immobilized anti-CD96 candidates. Isotype control antibody was tested immobilized at 10 μg/mL (white bars). The results are expressed as the % of CD25 positive cells within the CD3+/CD4+ and CD3+/CD8+ T cell populations. On the grafts, dashed lines mark the % of CD25+ cells after stimulation by OKT3 only and plain lines mark the % of CD25+ cells after stimulation by OKT3 and isotype control antibody.

FIG. 13: Co-stimulation of CD4+ and CD8+ T cell proliferation by anti-hCD96 candidates BL006-4-20. PBMC from a healthy donor were labelled with CFSE and cultured in the presence of 0.1, 1 or 10 ng/mL OKT3 on plates coated with 10 μg/mL anti-CD96 BL006-4-20 antibody or control isotype antibody. After 3 days, the cells were collected and the dilution of CFSE was measured by FCM on CD4+ and CD8+ T cells. Vertical dashed lines mark threshold for CFSE dilution on each set of histograms.

FIG. 14: Co-stimulation of CD4+ and CD8+ T cell proliferation by anti-hCD96 candidates. PBMC from 2 healthy donors were labelled with CFSE and cultured in the presence of 0.1 ng/mL OKT3 (white bars) on plates coated with 10 μg/mL anti-CD96 antibodies (grey bars), reference anti-CD96 antibody (NK92.39, hatched bars) or control isotype antibodies (white bars). After 3 days, the cells were collected, the dilution of CFSE was measured by FCM on CD4+ and CD8+ T cells and the % of dividing cells were determined. Results are expressed as mean+/−SD of triplicates from 2 experiments with 2 independent donors (Donor #2 and #3). On the grafts, dashed lines mark the % of dividing cells after stimulation by OKT3 only and plain lines mark the % of dividing cells after stimulation by OKT3 and isotype control antibody.

FIG. 15: Co-stimulation of CD4+ and CD8+ T cell proliferation by anti-hCD96 candidates with sub-optimal concentration of OKT3. PBMC from a healthy donor were stained with CFSE and cultured on plastic-immobilized anti-CD96 mAbs, isotype control, or reference anti-CD96 antibody (NK92.39) (all at 10 μg/mL), in the presence of 0.1 ng/mL soluble OKT3. The % of CD4+ and CD8+ dividing T cells was measured by FCM at day 3, 4 and 5 after initiation of the culture by measuring the % of cells showing dilution of CFSE. Results are expressed as mean+/−SD of triplicates. Error bars not appearing on the graft indicate that the error bar is smaller than the symbol.

FIG. 16: Co-stimulation of CD4+ T cell proliferation by anti-CD96 candidate antibodies. PBMC from a healthy donor were stained with CFSE and cultured in the presence of 0.1, 0.3 or 1 ng/mL soluble OKT3 on plastic-immobilized anti-CD96 mAbs (grey bars) or isotype control murine IgG2a (white bar) mAb (10 μg/mL), or with soluble anti-CD28 mAb (hatched bar), reference anti-CD96 (NK92.39, hatched bar) or isotype control murine IgG 1 (white bar) mAb (all at 10 μg/mL). The % of CD4+ dividing T cells was measured by FCM at day 4 after initiation of the culture by measuring the % of cells showing dilution of CFSE. Upper panels show the % of dividing cells, median panels show the intensity of the CFSE measured for each culture condition and the lower panels are the summary of the results obtained for the 3 OKT3 concentrations tested. To simplify the reading of the lower panels, results from the various anti human CD96 candidates are shown on 4 different panels. Results are expressed as mean+/−SD of triplicates. Error bars not appearing on the graft indicate that the error bar is smaller than the symbol.

FIG. 17: Co-stimulation of CD8+ T cell proliferation by anti-CD96 candidate antibodies. PBMC from a healthy donor were stained with CFSE and cultured in the presence of 0.1, 0.3 or 1 ng/mL soluble OKT3 on plastic-immobilized anti-CD96 mAbs (grey bars) or isotype control murine IgG2a (white bar) mAb (10 μg/mL), or with soluble anti-CD28 mAb (hatched bars), reference anti-CD96 (NK92.39, hatched bar) or isotype control murine IgG 1 (white bar) mAb (all at 10 μg/mL). The % of CD8+ dividing T cells was measured by FCM at day 4 after initiation of the culture by measuring the % of cells showing dilution of CFSE. Upper panels show the % of dividing cells, median panels show the intensity of the CFSE measured for each culture condition and the lower panels are the summary of the results obtained for the 3 OKT3 concentrations tested. To simplify the reading of the lower panels, results from the various anti human CD96 candidates are shown on 4 different panels. Results are expressed as mean+/−SD of triplicates. Error bars not appearing on the graft indicate that the error bar is smaller than the symbol.

FIG. 18: Co-stimulation of purified T cells by anti-CD96 candidate antibodies. PBMCs from healthy donors were used to purify T cells by magnetic cell sorting. Purified CD4+ and CD8+ T cells were stained with CFSE and cultured with or without 2 μL/mL tetrameric CD3/CD28 antibodies (ImmunoCult™) on plastic immobilized anti CD96 mAbs (grey bars) or isotype control murine IgG2a (white bar) mAb (10 μg/mL). The % of CD4+ or CD8+ dividing T cells was measured by FCM at day 5 after initiation of the culture by measuring the % of cells showing dilution of CFSE. Results are expressed as mean+/−SD of triplicates obtained from 2 donors in 2 independent experiments. On the grafts, dashed lines mark the % of dividing cells after stimulation by ImmunoCult™ only and plain lines mark the % of dividing cells after stimulation by ImmunoCult™+isotype control antibody.

FIG. 19: Co-stimulation of CD4+ and CD8+ T cell proliferation by anti-hCD96 candidates with sub-optimal concentration of OKT3 and blockade of CD226 or CD155. PBMC from a healthy donor were stained with CFSE and cultured on plastic-immobilized anti-CD96 mAbs or isotype control (10 μg/mL), in the presence of 1 ng/mL soluble OKT3 and 25 μg/mL of soluble blocking antibodies to CD226 (DX11) or CD155 (SKII.4) or isotype control where indicated. CD4+ and CD8+ cell division was estimated by FCM at day 4 after initiation of the culture by measuring the median of fluorescence intensity (MedFI) of CFSE on CD4+ or CD8+ T cells. Results are expressed as mean+/−SD of triplicates. On the grafts, dashed lines mark the CFSE MedFI after stimulation by OKT3 only and plain lines mark the CFSE MedFI after stimulation by OKT3+isotype control antibody.

FIG. 20: Temporal requirement of the CD96 signal. PBMCs from a healthy donor were used to purify T cells by magnetic cell sorting. Purified CD4+ and CD8+ T cells were stained with CFSE and cultured with 2 μL/mL tetrameric CD3/CD28 antibodies (white bars, ImmunoCult™) on plastic immobilized anti CD96 mAbs (grey bars) or isotype control murine IgG2a (dotted bar) mAb (10 μg/mL), either simultaneously (DO) or at day 1, 2 or 3 (D1, D2 and D3 respectively). The % of CD4+ or CD8+ dividing T cells was measured by FCM at day 5 after initiation of the culture by measuring the % of cells showing dilution of CFSE. Results are expressed as mean+/−SD of triplicates. On the grafts, dashed lines mark the % of dividing cells after stimulation by ImmunoCult™ only and plain lines mark the % of dividing cells after stimulation by ImmunoCult™+isotype control antibody.

FIG. 21: Co-stimulation of CD4+ and CD8+ T cell proliferation by soluble chimeric IgG1 anti-hCD96 candidates (CHG1) with sub-optimal concentrations of OKT3. PBMC from healthy donors were stained with CFSE and cultured in the presence of 0.1 or 0.3 ng/mL soluble OKT3 with soluble anti-CD96 mAbs (grey plain bars) or 2 different isotype controls (white bar) mAb (1 μg/mL). For each CHG1 clone evaluated in this experiment, a corresponding Fc-silent version (grey hatched bars, CHS1) has been tested. After 4 days, the cells were collected, the dilution of CFSE was measured by FCM on CD4+ and CD8+ T cells and the % of dividing cells were determined. Results are expressed as mean+/−SD of triplicates obtained from 2 to 4 different healthy donors. On the grafts, dashed lines mark the % of dividing cells after stimulation by OKT3 only and plain lines mark the % of dividing cells after stimulation by OKT3 and isotype control antibody.

FIG. 22: Co-stimulation of tumor infiltrating lymphocytes (TILs) proliferation by plate bound chimeric IgG1 anti-hCD96 candidate with sub-optimal concentrations of Immunocult™. Surgically removed head and neck tumors from 2 patients were processed for TILs extraction. Recovered TILs were cultured in the presence of increasing concentrations of Immunocult™ (white bars). A sub-optimal concentration of Immunocult (3 μL/mL) was used to test the co-stimulating effect of plate bound candidate BL006-19-134CHG1 (5 μg/mL). After 5 days of co-culture, TILs proliferation was assessed by 3[H] Thymidine incorporation (16 hours pulse). Results are expressed as mean+/−SD of triplicates. On the graphs, dashed horizontal lines mark the CPM after stimulation by 3 μL/mL OKT3 Immunocult™ only.

### DETAILED DESCRIPTION

The present invention provides CD96-binding agents that activate T cells. In a preferred embodiment, the CD96-binding agents are antibodies or fragments thereof. However alternative CD96-binding agents are also within the scope of the invention, e.g. aptamers, oligonucleotides, mimetics, peptides or small molecular weight compounds, or combinations thereof, which bind to CD96, preferably human CD96. In one embodiment, the CD96-binding agent is a soluble receptor for CD96, e.g. a soluble fragment of CD155 (in monomeric or oligomeric form) that binds to CD96.

The CD96 binding agents stimulate activation and/or proliferation of T cells. By this it is meant that T cells (or a subset thereof) show increased activation and/or proliferation in the presence of the CD96-binding agent, either in vitro or in vivo. T cell activation and/or proliferation can be measured by standard assays, as described e.g. in the examples below. For instance, T cell activation may be measured by the increased expression of a T cell activation marker, e.g. CD25, CD69, CD137 or CD107a, and T cell proliferation may be measured by the analysis of dye dilution in dividing T cells previously labelled with a fluorescent dye, e.g. CFSE. In some embodiments the T cells may be e.g. helper or cytotoxic T cells (CD4+ or CD8+ T cells).

The increased activation and/or proliferation of T cells may occur either in the presence of the CD96-binding agent alone, as for example in an in vivo tumor environment, or in the presence of the CD96-binding agent in combination with a further agent. For instance, in one embodiment the CD96-binding agent co-stimulates T cell activation/proliferation, i.e. the CD96-binding agent stimulates T cell activation and/or proliferation in combination with a further agent (a T cell co-stimulatory agent).

Preferably T cell activation and/or proliferation (e.g. as determined by expression of a T cell activation marker and/or fluorescent dye dilution) is increased by at least 5%, at least 10%, at least 15%, at least 20%, at least 25%, at least 30%, at least 40%, at least 50%, at least 75% or at least 100% in the presence of the CD96 binding agent (e.g. in the presence of the CD96-binding agent and T cell co-stimulatory agent), as compared to T cell activation and/or proliferation in the absence of the CD96-binding agent (e.g. in the presence of a T cell co-stimulatory agent alone). Preferably the CD96-binding agent stimulates T cell activation and/or proliferation with an EC50 value of less than 100 nM, less than 10 nM, less than 5 nM or less than 1 nM.

In a preferred embodiment, the CD96-binding agent is a CD96 agonist. A CD96 agonist is an agent which binds to CD96 (preferably human CD96) and stimulates signaling via CD96, thereby promoting T cell activation and/or proliferation. For instance, the agonist may bind to CD96 and produce a conformational change in the CD96 protein, thereby promoting intracellular signaling via CD96. In some embodiments, the CD96-binding agent may induce dimerization or multimerization of CD96, e.g. binding of the agent to CD96 may result in association of CD96 into homodimers or multimers, or association of CD96 into heterodimers or multimers in combination with a further cell surface protein such as CD226. Dimerization or multimerization of CD96 may be detected using standard techniques, e.g. using fluorescence resonance energy transfer (FRET) as described in the examples below.

In some embodiments, the CD96-binding agent is an antibody, preferably a monoclonal antibody. In one aspect, the present invention is directed to mouse, chimeric, humanized or fully human monoclonal antibodies capable of binding specifically to CD96, and cell lines that produce such antibodies.

In some other aspects, the CD96-binding agent is an antibody mimetic or an antibody mimetic fragment. Antibody mimetics are organic compounds or scaffold proteins that can specifically bind to various antigens. Examples of antibody mimetics include affibody molecules, affilins, affimers, affitins, alphabodies, anticalins, avimers, DARPins, fynomers, monobodies, aptamers, beta-hairpin mimetics, non-immunoglobulin scaffolds, or fusion proteins.

The term “antibody” is used in the broadest sense and specifically covers monoclonal antibodies (including full length monoclonal antibodies), polyclonal antibodies, multi-specific antibodies (e.g., bi-specific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

“Native antibodies and immunoglobulins” are usually heterotetrameric glycoproteins of about 150,000 dalton, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia C, Novotny J, Bruccoleri R, and Karplus M, Domain association in immunoglobulin molecules. The packing of variable domains, J Mol. Biol. 186:651-63 (1985); Novotny J and Haber E., Structural invariants of antigen binding: comparison of immunoglobulin VL-VH and VL-VL domain dimers., Proc. Natl. Acad. Sci. U.S.A. 82: 4592-96 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (for KABAT annotation see Kabat E. A. Sequences of Proteins of Immunological Interest, Fifth Edition, National Institutes of Health, Bethesda, Md. (1991) or for IMGT annotation, see http://www.imgt.org). The constant domains are not involved directly in the binding of an antibody to an antigen, but exhibit various effector functions, such as participation of the antibody in antibody-dependent cellular toxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen recognition and binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that of a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv, see Plückthun A. Antibodies from Escherichia coli, in “The Pharmacology of Monoclonal Antibodies”, by Rosenburg and Moore eds., Springer-Verlag, New York, vol. 113, pp. 269-315 (1994).

The Fab fragment also contains the constant domain of the light chain and the first constant domain of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

There are five major classes of immunoglobulins: IgA, IgD, IgE, IgG, and IgM, and several of these can be further divided into subclasses (isotypes), e.g., IgG1, lgG2, lgG3, lgG4, IgA1, and lgA2. The heavy-chain constant domains that correspond to the different classes of immunoglobulins are called α, δ, ε, γ, and μ, respectively. The subunit structures and three-dimensional configurations of different classes of immunoglobulins are well known.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture or mammalian cell lines, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made in an immortalized B cell or hybridoma thereof or may be made by recombinant DNA methods.

The monoclonal antibodies herein include hybrid and recombinant antibodies produced by splicing a variable (including hypervariable) domain of an anti-CD96 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)2, and Fv), so long as they exhibit the desired biological activity.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

The CD96-binding agents (e.g. antibodies) of the present invention may be used in isolated form. An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In some embodiments, the antibody will be purified (1) to greater than 75% by weight of antibody as determined by the Lowry method, and most preferably more than 80%, 90% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

In one aspect, the present invention provides mouse and chimeric or humanized mouse-human monoclonal antibodies binding to human CD96 with a dissociation constant (K_{D}) in the range from 0.01 to 50 nM, e.g. 0.18 to 42.3 nM. Some of the antibodies co-stimulate the activation and the proliferation of human CD4+ and CD8+ T cells. These agonistic antibodies find use for stimulating immune responses in patients with cancer or infectious diseases. Some of the disclosed antibodies disrupt or inhibit the CD96-CD155 interaction and could have a functional role in the regulation of T cell function.

Anti-human CD96 (hCD96) antibodies were generated by immunization of mice upon priming with the DNA coding for hCD96 and boosting with a cell line displaying the human CD96. B cells isolated from spleens and lymph nodes were then screened by ISAAC (ImmunoSpot Array Assay on Chip) to isolate a large panel of antibodies binding to hCD96. Thanks to the large diversity of antibodies generated, anti-hCD96 candidates with novel biological activities not previously disclosed were identified.

Nineteen anti-human CD96 antibodies have been produced and characterized in vitro. Nine candidates strongly inhibited the interaction between CD96 and CD155 with an IC50 in the range of 5.92 to 19.42 nM and with maximal inhibition capacity of 98 to 100%. Three mAbs including candidate BL006-4-20 inhibited CD96/CD155 interaction with a lower maximal inhibition capacity inferior to 50% (partial inhibitors).

Anti-CD96 candidates with strong co-stimulating activity on human CD4+ and CD8+ T cell activation and proliferation were identified. Thirteen candidates (see Table 4) displayed co-stimulation of peripheral blood T cells activated with sub-optimal concentrations of anti-CD3 antibody OKT3. Of importance, the benchmark anti-hCD96 NK92.39 antibody used by other groups (Fuchs et al., 2004; WO2015024060) did not show significant co-stimulating activity in the same in vitro assays.

Some of the anti-CD96 antibodies of the invention bind to human and rhesus CD96, while others are restricted to human. Cross-reactivity represents an advantage in terms of preclinical development, e.g. the ability to undergo efficacy and safety testing in the same model.

Thus, novel antibodies with specific activating activity on human CD96-expressing T cells have been identified in the present invention. These antibodies can, therefore, be used as therapeutics to stimulate T cell responses either alone or in combination with checkpoint inhibitors or other therapeutic drugs to treat cancer.

TABLE 1
Selected candidates with candidate no, antibody name,
variable region of heavy chain (VH) SEQ ID NOs and
variable region of light chain (VL) SEQ ID NOs:

| | | Variable | Variable |
|---|---|---|---|
| | | Region of | Region of |
| | | Heavy chain | Light Chain |
| | | (herein also | (herein also |
| | | referred to | referred to |
| Candidate | | as “VH”) | as “VL”) |
| NO | Antibody name | SEQ ID NO | SEQ ID NO |
| 1 | BL006-4-20G5-20K5 | 229 | 230 |
| 2 | BL006-4-52G6-52K7 | 231 | 232 |
| 3 | BL006-4-61G5-61K1 | 233 | 234 |
| 4 | BL006-8-3G9-3K4 | 235 | 236 |
| 5 | BL006-11-5G1-5K3 | 237 | 238 |
| 6 | BL006-19-14G3-14K1 | 239 | 240 |
| 7 | BL006-19-134G5-134K4 | 241 | 242 |
| 8 | BL006-19-183G3-183K4 | 243 | 244 |
| 9 | BL006-2-19G13-19K15 | 245 | 246 |
| 10 | BL006-4-31G1-31K3 | 247 | 248 |
| 11 | BL006-9-1G1-1K1 | 249 | 250 |
| 12 | BL006-9-15G1-15K5 | 251 | 252 |
| 13 | BL006-19-21G1-21K1 | 253 | 254 |
| 14 | BL006-19-29G5-29K1 | 255 | 256 |
| 15 | BL006-19-55G10-55K7 | 257 | 258 |
| 16 | BL006-19-190G4-190K6 | 259 | 260 |
| 17 | BL006-19-352G9-352K8 | 261 | 262 |
| 18 | BL006-19-363G1-363K1 | 263 | 264 |
| 19 | BL006-19-370G9-370K16 | 265 | 266 |

TABLE 2
Selected candidates with candidate no, antibody name, CDRs of heavy chain (VH)
SEQ ID NOs and CDRs of light chain (VL) SEQ ID NOs (RABAT annotations):

| | | SEQ ID | SEQ ID | SEQ ID | SEQ ID | SEQ ID | SEQ ID |
|---|---|---|---|---|---|---|---|
| | | NO of | NO of | NO of | NO of | NO of | NO of |
| | | CDR1 of | CDR2 of | CDR3 of | CDR1 of | CDR2 of | CDR3 of |
| NO | Antibody name | VH | VH | VH | VL | VL | VL |
| 1 | BL006-4-20G5-20K5 #1 | 1 | 2 | 3 | 7 | 8 | 9 |
| 2 | BL006-4-52G6-52K7 #4 | 13 | 14 | 15 | 19 | 20 | 21 |
| 3 | BL006-4-61G5-61K1 #7 | 25 | 26 | 27 | 31 | 32 | 33 |
| 4 | BL006-8-3G9-3K4 #6 | 37 | 38 | 39 | 43 | 44 | 45 |
| 5 | BL006-11-5G1-5K3 #5 | 49 | 50 | 51 | 55 | 56 | 57 |
| 6 | BL006-19-14G3-14K1 #8 | 61 | 62 | 63 | 67 | 68 | 69 |
| 7 | BL006-19-134G5-134K4 #2 | 73 | 74 | 75 | 79 | 80 | 81 |
| 8 | BL006-19-183G3-183K4 #3 | 85 | 86 | 87 | 91 | 92 | 93 |
| 9 | BL006-2-19G13-19K15 | 97 | 98 | 99 | 103 | 104 | 105 |
| 10 | BL006-4-31G1-31K3 | 109 | 110 | 111 | 115 | 116 | 117 |
| 11 | BL006-9-1G1-1K1 | 121 | 122 | 123 | 127 | 128 | 129 |
| 12 | BL006-9-15G1-15K5 | 133 | 134 | 135 | 139 | 140 | 141 |
| 13 | BL006-19-21G1-21K1 | 145 | 146 | 147 | 151 | 152 | 153 |
| 14 | BL006-19-29G5-29K1 | 157 | 158 | 159 | 163 | 164 | 165 |
| 15 | BL006-19-55G10-55K7: | 169 | 170 | 171 | 175 | 176 | 177 |
| 16 | BL006-19-190G4-190K6 | 181 | 182 | 183 | 187 | 188 | 189 |
| 17 | BL006-19-352G9-352K8 | 193 | 194 | 195 | 199 | 200 | 201 |
| 18 | BL006-19-363G1-363K1 | 205 | 206 | 207 | 211 | 212 | 213 |
| 19 | BL006-19-370G9-370K16 | 217 | 218 | 219 | 223 | 224 | 225 |

TABLE 3
Selected candidates with candidate no, antibody name, CDRs of heavy chain
(VH) SEQ ID NOs and CDRs of light chain (VL) SEQ ID NOs (IMGT annotations):

| | | SEQ ID | SEQ ID | SEQ ID | SEQ ID | SEQ ID | SEQ ID |
|---|---|---|---|---|---|---|---|
| | | NO of | NO of | NO of | NO of | NO of | NO of |
| | | CDR1 of | CDR2 of | CDR3 of | CDR1 of | CDR2 of | CDR3 of |
| NO | Antibody name | VH | VH | VH | VL | VL | VL |
| 1 | BL006-4-20G5-20K5 #1 | 4 | 5 | 6 | 10 | 11 | 12 |
| 2 | BL006-4-52G6-52K7 #4 | 16 | 17 | 18 | 22 | 23 | 24 |
| 3 | BL006-4-61G5-61K1 #7 | 28 | 29 | 30 | 34 | 35 | 36 |
| 4 | BL006-8-3G9-3K4 #6 | 40 | 41 | 42 | 46 | 47 | 48 |
| 5 | BL006-11-5G1-5K3 #5 | 52 | 53 | 54 | 58 | 59 | 60 |
| 6 | BL006-19-14G3-14K1 #8 | 64 | 65 | 66 | 70 | 71 | 72 |
| 7 | BL006-19-134G5-134K4 #2 | 76 | 77 | 78 | 82 | 83 | 84 |
| 8 | BL006-19-183G3-183K4 #3 | 88 | 89 | 90 | 94 | 95 | 96 |
| 9 | BL006-2-19G13-19K15 | 100 | 101 | 102 | 106 | 107 | 108 |
| 10 | BL006-4-31G1-31K3 | 112 | 113 | 114 | 118 | 119 | 120 |
| 11 | BL006-9-1G1-1K1 | 124 | 125 | 126 | 130 | 131 | 132 |
| 12 | BL006-9-15G1-15K5 | 136 | 137 | 138 | 142 | 143 | 144 |
| 13 | BL006-19-21G1-21K1 | 148 | 149 | 150 | 154 | 155 | 156 |
| 14 | BL006-19-29G5-29K1 | 160 | 161 | 162 | 166 | 167 | 168 |
| 15 | BL006-19-55G10-55K7: | 172 | 173 | 174 | 178 | 179 | 180 |
| 16 | BL006-19-190G4-190K6 | 184 | 185 | 186 | 190 | 191 | 192 |
| 17 | BL006-19-352G9-352K8 | 196 | 197 | 198 | 202 | 203 | 204 |
| 18 | BL006-19-363G1-363K1 | 208 | 209 | 210 | 214 | 215 | 216 |
| 19 | BL006-19-370G9-370K16 | 220 | 221 | 222 | 226 | 227 | 228 |

The antibodies of the invention may have at least one of the following characteristics: stimulation (including co-stimulation) of T cell activation and/or proliferation, binding to (e.g. human or rhesus) CD96, agonism of (e.g. human) CD96, induction of dimerization or multimerization on (e.g. human) CD96 on T cells, inhibition of binding of (e.g. human) CD155 to CD96, and inhibition of tumor growth, e.g. in xenograft mouse models.

In the following paragraphs, the candidate antibodies are referred to by the names given in Table 1 to 3 which in some cases may be abbreviated for convenience, e.g. BL006-4-31G1-31K3 may be referred to as BL006-4-31, BL006-19-183G3-183K4 may be referred to as BL006-19-183 and so on. In some embodiments, the antibody is one of the candidates described in Tables 1 to 3 or a variant thereof, e.g. an antibody comprising one or more CDR sequences or variable regions from one of the above antibodies.

In a preferred embodiment, the antibody has a high affinity for binding to human CD96. For instance, in one embodiment the antibody binds to human CD96 (e.g. variant 2, short isoform, SEQ ID NO: 268) e.g. with an EC50 value of less than 10 nM. The EC50 value may, for example be determined by flow cytometry analysis on CHO cells transfected with human CD96 variant 2. 11 candidates were found to bind strongly to CD96 (BL006-4-31, BL006-19-183, BL006-19-190, BL006-19-134, BL006-19-21, BL006-19-55, BL006-19-352, BL006-19-363, BL006-19-370, BL006-19-14, BL006-19-29). Thus, in some embodiments, the antibody is one of the above candidates or a variant thereof, e.g. an antibody comprising one or more CDR sequences or variable regions from one of the above antibodies. Preferred antibodies comprise one or more CDR sequences (e.g. 3 heavy chain and 3 light chain CDR sequences) or a heavy and/or light chain variable domain selected from SEQ ID NO:s: 109-120, 247, 248 (BL006-4-31); 85-96, 243, 244 (BL006-19-183); 181-192, 259, 260 (BL006-19-190); 73-84, 241, 242 (BL006-19-134); 145-156, 253, 254 (BL006-19-21); 169-180, 257, 258 (BL006-19-55); 193-204, 261, 262 (BL006-19-352); 205-216, 263, 264 (BL006-19-363); 217-228, 265, 266 (BL006-19-370); 61-72, 239, 240 (BL006-19-14); and 157-168, 255, 256 (BL006-19-29).

In another embodiment, the antibody has a high affinity for the long form of human CD96, i.e. variant 1, SEQ ID NO: 267, e.g. the antibody binds with an EC50 value of less than 10 nM determined by flow cytometry analysis on CHO cells transfected with human CD96 variant 1. 8 candidates were also found to strongly bind to the long form of human CD96 (CD96v1) (BL006-19-183, BL006-19-14, BL006-19-29, BL006-2-19, BL006-11-5, BL006-4-61, BL006-9-1, BL006-8-3, BL006-9-15). Thus, in some embodiments, the antibody is one of the above candidates or a variant thereof, e.g. an antibody comprising one or more CDR sequences or variable regions from one of the above antibodies. Preferred antibodies comprise one or more CDR sequences (e.g. 3 heavy chain and 3 light chain CDR sequences) or a heavy and/or light chain variable domain selected from SEQ ID NO:s: 85-96, 243, 244 (BL006-19-183); 61-72, 239, 240 (BL006-19-14); 157-168, 255, 256 (BL006-19-29); 97-108, 245, 246 (BL006-2-19); 49-60, 237, 238 (BL006-11-5); 25-36, 233, 234 (BL006-4-61); 121-132, 249, 250 (BL006-9-1); 37-48, 235, 236 (BL006-8-3); 133-144, 251, 252 (BL006-9-15).

In another embodiment, the antibody binds strongly to human T cells and/or NK cells. Thus, in some embodiments, the antibody is one of the above candidates or a variant thereof, e.g. an antibody comprising one or more CDR sequences or variable regions from one of the above antibodies. Preferred antibodies comprise one or more CDR sequences (e.g. 3 heavy chain and 3 light chain CDR sequences) or a heavy and/or light chain variable domain selected SEQ ID NO:s: 1-12, 229, 230 (BL006-4-20G5), 13-24, 231, 232 (BL006-4-52G6), 109-120, 247, 248 (BL006-4-31); 85-96, 243, 244 (BL006-19-183); 181-192, 259, 260 (BL006-19-190); 73-84, 241, 242 (BL006-19-134); 145-156, 253, 254 (BL006-19-21); 169-180, 257, 258 (BL006-19-55); 193-204, 261, 262 (BL006-19-352); 205-216, 263, 264 (BL006-19-363); 217-228, 265, 266 (BL006-19-370); 61-72, 239, 240 (BL006-19-14); and 157-168, 255, 256 (BL006-19-29).

In another embodiment, the antibody has a high affinity for rhesus CD96, e.g. the antibody binds with an EC50 value of less than 10 nM determined by flow cytometry analysis in CHO cells transfected with rhesus CD96 (SEQ ID NO: 272). 8 antibodies strongly recognized rhesus CD96 (BL006-4-31, BL006-19-352, BL006-19-14, BL006-19-29, BL006-4-52, BL006-2-19, BL006-4-61, BL006-9-1). Thus, in some embodiments, the antibody is one of the above candidates or a variant thereof, e.g. an antibody comprising one or more CDR sequences or variable regions from one of the above antibodies. Preferred antibodies comprise one or more CDR sequences (e.g. 3 heavy chain and 3 light chain CDR sequences) or a heavy and/or light chain variable domain selected from SEQ ID NO:s: 109-120, 247, 248 (BL006-4-31); 193-204, 261, 262 (BL006-19-352); 61-72, 239, 240 (BL006-19-14); 157-168, 255, 256 (BL006-19-29); 13-24, 231, 232 (BL006-4-52); 97-108, 245, 246 (BL006-2-19); 25-36, 233, 234 (BL006-4-61); 121-132, 249, 250 (BL006-9-1).

Thus, in preferred embodiments, the antibody binds to one or more forms of CD96, e.g. to human CD96 variants 1 and 2 or to human CD96 and rhesus CD96. Preferred antibodies that cross-react with human and rhesus CD96 comprise one or more CDR sequences (e.g. 3 heavy chain and 3 light chain CDR sequences) or a heavy and/or light chain variable domain selected from SEQ ID NOs: 61-72, 239, 240 (BL006-19-14); 109-120, 247, 248 (BL006-4-31); 157-168, 255, 256 (BL006-19-29); and 193-204, 261, 262 (BL006-19-352).

In another embodiment, the antibody inhibits the binding of CD155 to CD96. For instance, the antibody may inhibit the binding of hCD155 to hCD96v2 expressed on CHO cells, as determined by flow cytometry, with an IC50 value of less than 20 nM. 9 candidates strongly inhibited the binding of hCD155 to hCD96v2 with IC50 values ranging from 5.9 to 19.4 nM (candidates BL006-4-31, BL006-19-21, BL006-19-183, BL006-19-190, BL006-19-134, BL006-19-55, BL006-19-352, BL006-19-363, BL006-19-370). Those 9 anti-CD96 candidates were similar to the clone 628211 and NK92.39 antibodies in this assay. Thus, in some embodiments, the antibody is one of the above candidates or a variant thereof, e.g. an antibody comprising one or more CDR sequences or variable regions from one of the above antibodies. Preferred antibodies comprise one or more CDR sequences (e.g. 3 heavy chain and 3 light chain CDR sequences) or a heavy and/or light chain variable domain selected from SEQ ID NO:s: 109-120, 247, 248 (BL006-4-31); 145-156, 253, 254 (BL006-19-21); 85-96, 243, 244 (BL006-19-183); 181-192, 259, 260 (BL006-19-190); 73-84, 241, 242 (BL006-19-134); 169-180, 257, 258 (BL006-19-55); 193-204, 261, 262 (BL006-19-352); 205-216, 263, 264 (BL006-19-363); 217-228, 265, 266 (BL006-19-370).

In an alternative embodiment, the antibody partially inhibits the binding of CD155 to CD96. By “partially inhibits” or “partial inhibitor” it is meant that the antibody inhibits less than 100% of the available CD155/CD96 binding sites at an excess concentration of antibody, e.g. the antibody maximally inhibits less than 95%, less than 90%, less than 80%, less than 70%, less than 60% or less than 50% of CD155/CD96 binding. For instance, 3 candidates (BL006-4-20, BL006-19-14 and BL006-19-29) were found to be partial inhibitors of CD155 binding to CD96, with a maximal inhibition capacity inferior to 50%, but with a relative IC50 value between 2.67 and 5.24 nM. Thus, in some embodiments, the antibodies comprise one or more CDR sequences (e.g. 3 heavy chain and 3 light chain CDR sequences) or a heavy and/or light chain variable domain selected from SEQ ID NOs: 1-12, 229, 230 (BL006-4-20); 61-72, 239, 240 (BL006-19-14); and 157-168, 255, 256 (BL006-19-29).

In an alternative embodiment, the antibody does not inhibit the binding of CD155 to CD96. For instance, 6 candidates (BL006-4-52, BL006-4-61, BL006-11-5, BL006-8-3, BL006-9-1 and BL006-9-15) did not inhibit the CD96/CD155 interaction. Preferred antibodies comprise one or more CDR sequences (e.g. 3 heavy chain and 3 light chain CDR sequences) or a heavy and/or light chain variable domain selected from SEQ ID NOs: 13-24, 231, 232 (BL006-4-52); 25-36, 233, 234 (BL006-4-61); 49-60, 237, 238 (BL006-11-5); 37-48, 235, 236 (BL006-8-3); 121-132, 249, 250 (BL006-9-1) and 133-144, 251, 252 (BL006-9-15).

In another embodiment, the antibody does bind to the D1 domain of the CD96 protein. For instance, 1 candidate (BL006-19-183) binds to the D1 domain of CD96. Preferred antibody comprises one or more CDR sequences (e.g. 3 heavy chain and 3 light chain CDR sequences) or a heavy and/or light chain variable domain selected from SEQ ID Nos: 85-96, 243, 244 (BL006-19-183).

In another embodiment, the antibody binds to the D1 and D2 domains of the CD96 protein. For instance, 9 candidates (BL006-4-31, BL006-4-20, BL006-19-190, BL006-19-21, BL006-19-55, BL006-19-370, BL006-19-363, BL006-19-352 and BL006-19-134) bind to the D1 and D2 domains of CD96. Preferred antibodies comprise one or more CDR sequences (e.g. 3 heavy chain and 3 light chain CDR sequences) or a heavy and/or light chain variable domain selected from SEQ ID Nos: 109-120, 247, 248 (BL006-4-31); 1-12, 229, 230 (BL006-4-20); 181-192, 259, 260 (BL006-19-190); 145-156, 253, 254 (BL006-19-21); 169-180, 257, 258 (BL006-19-55); 217-228, 265, 266 (BL006-19-370); 205-216, 263, 264 (BL006-19-363); 193-204, 261, 262 (BL006-19-352) and 73-84, 241, 242 (BL006-19-134).

In another embodiment, the antibody binds to the D2 and D3 domains of the CD96 protein. For instance, 2 candidates (BL006-19-14 and BL006-19-29) bind to the D2 and D3 domains of CD96. Preferred antibodies comprise one or more CDR sequences (e.g. 3 heavy chain and 3 light chain CDR sequences) or a heavy and/or light chain variable domain selected from SEQ ID Nos: 61-72, 239, 240 (BL006-19-14) and 157-168, 255, 256 (BL006-19-29).

In another embodiment, the antibody binds to the D3 domains of the CD96 protein. For instance, 2 candidates (BL006-4-52 and BL006-2-19) bind to the D3 domain of CD96. Preferred antibodies comprise one or more CDR sequences (e.g. 3 heavy chain and 3 light chain CDR sequences) or a heavy and/or light chain variable domain selected from SEQ ID Nos: 13-24, 231, 232 (BL006-4-52) and 97-108, 245, 246 (BL006-2-19).

In another embodiment, the antibody binds to the D4 domain of the CD96 protein. For instance, 5 candidates (BL006-4-61, BL006-8-3, BL006-9-1, BL006-9-15 and BL006-11-5) bind to the D4 domain of CD96, but did not show T cell activation or proliferation. Preferred antibodies comprise one or more CDR sequences (e.g. 3 heavy chain and 3 light chain CDR sequences) or a heavy and/or light chain variable domain selected from SEQ ID Nos: 25-36, 233, 234 (BL006-4-61); 37-48, 235, 236 (BL006-8-3); 121-132, 249, 250 (BL006-9-1); 133-144, 251, 252 (BL006-9-15) and 49-60, 237, 238 (BL006-11-5).

In another embodiment, the antibody according to anyone of the embodiments of the present invention is capable of co-activating the proliferation of T cells and bind an epitope either present in the D1 domain, or in the D1 and D2 domains, or in the D2 and D3 domains or in the D3 domain. For instance, such antibody may be capable of co-activating CD4+ and/or CD8+ T cells with a suboptimal concentration of a T cell stimulatory or proliferation agent, e.g. an anti-CD3 antibody. The percentage of activated cells may be measured e.g. by measuring increased expression of a T cell marker such as CD25, the dilution of CFSE staining and/or the incorporation of ³[H]-thymidine that are correlated to the number of dividing cells. Preferably the co-stimulation activity of the antibody is observed at low concentrations of anti-CD3 antibody (e.g. 0.1 ng/ml or below). BL006-4-31, BL006-4-20, BL006-19-14, BL006-19-190, BL006-19-21, BL006-19-55, BL006-19-370, BL006-19-363, BL006-19-352, BL006-19-29, BL006-19-183, BL006-19-134, and BL006-4-52 were found to show T cell stimulatory activity. Thus, in some embodiments, the antibody is a chimeric, humanized or engineered version of one of the above candidate. In another embodiment, the antibody is used as a soluble form. Thus, in some embodiments, the antibody is one of the above candidates or a variant thereof, e.g. an antibody comprising one or more CDR sequences or variable regions from one of the above antibodies. Preferred antibodies comprise one or more CDR sequences (e.g. 3 heavy chain and 3 light chain CDR sequences) or a heavy and/or light chain variable domain selected from SEQ ID NO:s: 109-120, 247, 248 (BL006-4-31); 1-12, 229, 230 (BL006-4-20); 61-72, 239, 240 (BL006-19-14); 181-192, 259, 260 (BL006-19-190); 145-156, 253, 254 (BL006-19-21); 169-180, 257, 258 (BL006-19-55); 217-228, 265, 266 (BL006-19-370); 205-216, 263, 264 (BL006-19-363); 193-204, 261, 262 (BL006-19-352); 157-168, 255, 256 (BL006-19-29); 85-96, 243, 244 (BL006-19-183); 73-84, 241, 242 (BL006-19-134); and 13-24, 231, 232 (BL006-4-52).

Preferred candidates are one or more of the antibodies defined above, including humanized and further engineered antibodies derived therefrom. The CDRs (in KABAT- (Table 2) and IMGT- (Table 3) annotations) and variable regions (Table 1) of exemplary antibodies (referred herein usually by candidate NOs or antibody name) are provided (IMGT annotations preferred). Antibodies of interest include these provided combinations, as well as fusions of the variable regions to appropriate constant regions or fragments of constant regions, e.g. to generate F(ab)′ antibodies. Variable regions of interest include at least one CDR sequence of the provided anti-CD96 antibodies, where a CDR may be 3, 4, 5, 6, 7, 8, 9, 10, 11, 12 or more amino acids. Alternatively, antibodies of interest include a variable region as set forth in the provided antibodies, or pairs of variable regions sequences as set forth herein. These antibodies may be full length antibodies, e.g. having a human immunoglobulin constant region of any isotype, e.g. lgG1, lgG2, lgG3, lgG4, IgA. Preferably the antibody is an IgG1, i.e. the antibody comprises a human IgG1 constant region such as e.g. a Fc part that enhances the agonism and other effector functions of the antibodies of the invention (Zhang D., Goldberg M. V. and Chiu M. L., Fc engineering approaches to enhance the agonism and effector functions of anti-OX40 antibody, The journal of biological chemistry, 291(53):27134-27146 (2016); Saxena A. and Wu, D., Advances in therapeutic Fc engineering—Modulation of IgG-associated effector functions and serum half-life, Frontiers in Immunology, 12(7):580 (2016)). Other preferred antibodies of the invention are antibodies conferring multimerization of the antibodies.

TABLE 4
Summary of anti-human CD96 candidate properties

| | | Binding to | Binding to | Binding to | Inhibition of | Binding to | T cell |
|---|---|---|---|---|---|---|---|
| | | hCD96 | hCD96 | rhesus | CD96/CD155 | human | activation/ |
| NO | Antibody name | short form | long form | CD96 | interaction | T cells | proliferation |
| 1 | BL006-4-20G5-20K5 #1 | + | +/− | − | Yes, Partial | ++ | Yes |
| 2 | BL006-4-52G6-52K7 #4 | + | + | ++ | No | + | Yes |
| 3 | BL006-4-61G5-61K1 #7 | + | ++ | ++ | No | + | No |
| 4 | BL006-8-3G9-3K4 #6 | + | ++ | − | No | + | No |
| 5 | BL006-11-5G1-5K3 #5 | + | ++ | − | No | +/− | No |
| 6 | BL006-19-14G3-14K1 #8 | ++ | + | ++ | Yes, Partial | ++ | Yes |
| 7 | BL006-19-134G5-134K4 #2 | ++ | +/− | − | Yes | ++ | Yes |
| 8 | BL006-19-183G3-183K4 #3 | ++ | + | − | Yes | ++ | Yes |
| 9 | BL006-2-19G13-19K15 | +/− | ++ | ++ | nd | + | No |
| 10 | BL006-4-31G1-31K3 | ++ | + | ++ | Yes | ++ | Yes |
| 11 | BL006-9-1G1-1K1 | + | ++ | ++ | No | Nd | No |
| 12 | BL006-9-15G1-15K5 | +/− | ++ | − | No | +/− | No |
| 13 | BL006-19-21G1-21K1 | ++ | + | + | Yes | ++ | Yes |
| 14 | BL006-19-29G5-29K1 | ++ | + | ++ | Yes, Partial | ++ | Yes |
| 15 | BL006-19-55G10-55K7: | ++ | +/− | + | Yes | ++ | Yes |
| 16 | BL006-19-190G4-190K6 | ++ | +/− | − | Yes | ++ | Yes |
| 17 | BL006-19-352G9-352K8 | ++ | +/− | ++ | Yes | ++ | Yes |
| 18 | BL006-19-363G1-363K1 | ++ | +/− | − | Yes | ++ | Yes |
| 19 | BL006-19-370G9-370K16 | ++ | +/− | − | Yes | ++ | Yes |

−: no binding by FCM; +/−: weak binding by FCM; +: binding by FCM; ++ strong binding by FCM.

The CD96-binding agents (e.g. antibodies) may be coupled to a further active agent, e.g. in the form of a conjugate. The conjugate may be e.g. a heterogeneous molecule formed by the covalent attachment of one or more active agent (e.g. antibody fragment(s)) to one or more polymer molecule(s), wherein the heterogeneous molecule is water soluble, i.e. soluble in physiological fluids such as blood, and wherein the heterogeneous molecule is free of any structured aggregate. A conjugate of interest is PEG. In the context of the foregoing definition, the term “structured aggregate” refers to (1) any aggregate of molecules in aqueous solution having a spheroid or spheroid shell structure, such that the heterogeneous molecule is not in a micelle or other emulsion structure, and is not anchored to a lipid bilayer, vesicle or liposome; and (2) any aggregate of molecules in solid or insolubilized form, such as a chromatography bead matrix, that does not release the heterogeneous molecule into solution upon contact with an aqueous phase. Accordingly, the term “conjugate” as defined herein encompasses the aforementioned heterogeneous molecule in a precipitate, sediment, bio-erodible matrix or other solid capable of releasing the heterogeneous molecule into aqueous solution upon hydration of the solid.

The CD96-binding agent (e.g. antibody) may be epitope tagged. The term “epitope tagged” when used herein refers to an anti-CD96 antibody fused to an “epitope tag”. The epitope tag polypeptide has enough residues to provide an epitope against which an antibody can be made, yet is short enough such that it does not interfere with activity of the CD96 antibody. The epitope tag preferably is sufficiently unique so that the antibody specific for the epitope does not substantially cross-react with other epitopes. Suitable tag polypeptides generally have at least 6 amino acid residues and usually between about 8-50 amino acid residues (preferably between about 9-30 residues). Examples include the c-myc tag and the 8F9, 3C7, 6E10, G4, B7 and 9E10 antibodies thereto (Evan G. I., Lewis G. K., Ramsay G., et al. “Isolation of monoclonal antibodies specific for human c-myc proto-oncogene product”, Mol. Cell. Biol. 5(12):3610-3616 (1985)); and the Herpes Simplex virus glycoprotein D (gD) tag and its antibody (Paborsky L. R., Fendly B. M., Fisher K. L., et al. “Mammalian cell transient expression of tissue factor for the production of antigen”, Protein Engineering 3(6):547-553 (1990)).

Further labels may be attached to the CD96-binding agent. The word “label” when used herein refers to a detectable compound or composition which is conjugated directly or indirectly to the agent (e.g. antibody). The label may itself be detectable by itself (e.g., radioisotope labels or fluorescent labels) or, in the case of an enzymatic label, may catalyze chemical alteration of a substrate compound or composition which is detectable.

In some embodiments, the CD96-binding agent may be attached to a solid phase. By “solid phase” is meant a non-aqueous matrix to which the agent (e.g. antibody) of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

The CD96-binding agents (e.g. antibodies) disclosed herein can be used in the treatment of disease, particularly in mammals (e.g. humans). “Treatment” refers to both therapeutic treatment and prophylactic or preventative measures. Those in need of treatment include those already with the disorder as well as those in which the disorder is to be prevented. “Mammal” for purposes of treatment refers to any animal classified as a mammal, including humans, domestic and farm animals, and zoo, sports, or pet animals, such as dogs, horses, cats, cows, etc. Preferably, the mammal is a human.

The CD96-binding agents (e.g. monoclonal antibodies) of the invention can be used to promote T cell activation and/or proliferation, e.g. in the treatment of cancer and infectious diseases. For example, CD96-binding agent (e.g. antibody) compositions may be administered to increase T cell activation and/or proliferation in cancer immunotherapy.

In another embodiment, the anti-CD96 binding agent, may be used as an adjuvant to a prophylactic or therapeutic vaccine in order to further stimulate the T cell response to cancer or chronic infectious disease associated antigens. Examples of vaccines include but are not restricted to peptide vaccines, proteinous vaccines, viral-based vaccines, DNA-based vaccines, RNA-based vaccines, autologous dendritic cell-based vaccines, allogeneic tumor cell vaccines and dendritic-cell based vaccines (Melero et al. Therapeutic vaccines for cancer: an overview of clinical trials. Nature reviews. Clinical oncology. 11(9):509-524 (2014))

Pharmaceutical compositions for use in the treatment of cancer comprising the CD96-binding agent (e.g. a humanized or chimeric monoclonal antibody) of the invention and optionally pharmaceutically suitable excipients or carrier are also provided.

In a preferred embodiment, the CD96-binding agent (e.g. antibody) of the invention can be used in treating, delaying the progression of, preventing relapse of or alleviating a symptom of a cancer or other neoplastic condition, as a monotherapy, or in combinations with other anti-cancer agent(s) (combination therapy). As used herein, the terms “cancer” “neoplasm” and “tumor” are interchangeable. Examples of cancer include, without limitation, gastric cancer, breast cancer, lung cancer, ovarian cancer, cervical cancer, prostate cancer, bladder cancer, colorectal cancer, pancreatic cancer, liver cancer, renal cancer, thyroid cancer, brain cancer, head and neck cancer, hematological cancer, carcinoma, melanoma, leiomyoma, leiomyosarcoma, glioma, glioblastoma, etc. The “hematological cancer” refers to a cancer of the blood, and includes leukemia, lymphoma and myeloma among others. Solid tumors include, for example, gastric tumor, breast tumors, lung tumors, ovarian tumors, prostate tumors, bladder tumors, colorectal tumors, pancreatic tumors, liver tumors, kidney tumors, thyroid tumor, brain tumor, head and neck tumors, esophageal tumors and melanoma tumors, etc. Symptoms associated with cancers and other neoplastic disorders include, but are not limited to, inflammation, fever, general malaise, pain, loss of appetite, weight loss, edema, headache, fatigue, rash, anemia, muscle weakness and muscle fatigue.

The combination therapy can include one or more CD96-binding agents (e.g. antibodies) of the invention co-formulated with, and/or co-administered with, one or more additional therapeutic agents, e.g., chemotherapeutic or anti-neoplastic agents, such as cytokine and growth factor inhibitors, immunosuppressants, anti-inflammatory agents, metabolic inhibitors, enzyme inhibitors, and/or cytotoxic or cytostatic agents. The term “combination” in this context means that the agents are given substantially contemporaneously, either simultaneously or sequentially. Exemplary chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, saproin, tamoxifen, taxol, topotecan hydrochloride, vinblastine, vincristine and vinorelbine tartrate.

The CD96-binding agents (e.g. antibodies) of the invention can be combined with an effective dose of an agent that increases patient hematocrit, for example erythropoietin stimulating agents (ESA). Such agents are known and used in the art, including, for example, Aranesp®, Epogen® NF/Procrit® NF, Omontys®, Procrit®, etc.

In other embodiments, the CD96-binding agents (e.g. antibodies) of the invention can be combined with an effective dose of other antibodies that have been used in treatment of cancer including, without limitation the following FDA approved monoclonal antibodies: rituximab (Rituxan®, CD20: chimeric IgG1), trastuzumab (Herceptin®, HER2: chimeric IgG1), alemtuzumab (Campath®, CD52: humanized IgG1), ibritumomab tiuxetan (Zevalin®, CD20: murine, IgG1, radiolabeled (Yttrium 90), tositumomab-I-131 (Bexxar®: CD20, murine, IgG2a, radiolabeled (Iodine 131)), cetuximab (Erbitux®, EGFR: chimeric, IgG1), bevacizumab (Avastin®, VEGF: humanized, IgG4), panitumumab (Vectibix®, EGFR: human IgG2), ofatumumab (Arzerra®, CD20: human IgG1), ipilimumab (Yervoy®, CTLA-4: human IgG1), brentuximab vedotin (Adcetris®, CD30: chimeric, IgG1, drug-conjugate), pertuzumab (Perjeta®, HER2: humanized IgG1), adotrastuzumab emtansine (Kadcyla®, HER2: humanized, IgG1, drug-conjugate), obinutuzumab (Gazyva®, CD20: humanized and glycol-engineered), nivolumab and pembrolizumab (anti-PD-1s), etc. Trastuzumab targets the HER-2 antigen. This antigen is seen on 25% to 35% of breast cancers and on metastatic gastric cancers. Trastuzumab is approved for the treatment of HER2-overexpressing breast cancers and for HER2-overexpressing metastatic gastric and gastroesophageal junction adenocarcinoma. Cetuximab is used for the treatment of metastatic colorectal cancer, metastatic non-small cell lung cancer and head and neck cancer. Nivolumab and pembrolizumab have been recently approved to treat metastatic melanoma and non-small cell lung cancer. They are tested in clinical trials for lung cancer, renal-cell cancer, lymphoma and mesothelioma. Other cancer drug that are currently tested in clinical trials or researched may also be combined.

In some embodiments, the anti-CD96 antibody may be used in combination with a T cell stimulating or proliferation agent, e.g. an anti-CD3 antibody. In some other embodiments, the anti-CD96 antibody may be used in combination with a T cell co-stimulatory agent, e.g. an agent that binds to a T cell co-stimulatory molecule such as CD28. For instance, the T cell co-stimulatory agent may be an anti-CD28, an anti-ICOS, an anti-CD226, an anti-CD40, an anti-OX40, an anti-CD137, an anti-GITR, or an anti-CD27 antibody.

In some embodiments, the anti-CD96 antibody may be combined with a checkpoint inhibitor. For instance, the checkpoint inhibitor may be an inhibitor of the programmed death-1 (PD-1) pathway, e.g. an anti-PD1 antibody such as nivolumab, atezolizumab, avelumab or pembrolizumab. In another embodiment, the checkpoint inhibitor is an anti-cytotoxic T-lymphocyte-associated antigen 4 (CTLA4) antibody, e.g. ipilimumab or tremelimumab.

Immune checkpoints are inhibitory pathways that slow down or stop immune reactions and prevent excessive tissue damage from uncontrolled activity of immune cells. By “checkpoint inhibitor” is meant to refer to any small molecule chemical compound, antibody, nucleic acid molecule, or polypeptide, or fragment thereof, that inhibits the inhibitory pathways, allowing more extensive immune activity. In certain embodiments, the checkpoint inhibitor is an inhibitor of the programmed death-1 (PD-1) pathway, for example an anti-PD1 antibody, such as, but not limited to nivolumab, atezolizumab, avelumab or pembrolizumab. In other embodiments, the checkpoint inhibitor is an anti-cytotoxic T-lymphocyte-associated antigen (CTLA-4) antibody. In additional embodiments, the checkpoint inhibitor is targeted at another member of the CD28/CTLA4 Ig superfamily such as BTLA, LAGS, ICOS, PDL1 or KIR (Page D. B., Postow M. A., Callahan M. K., Allison J. P. and Wolchok, J, Immune modulation in cancer with antibodies, Annual Review of Medicine, 65:185-202 (2014)). In further additional embodiments, the checkpoint inhibitor is targeted at a member of the TNFR superfamily such as CD40, OX40, 4-1BB, GITR, or CD27. In some cases, targeting a checkpoint inhibitor is accomplished with an inhibitory antibody or similar molecule. In other cases, it is accomplished with an agonist for the target; examples of this class include the stimulatory targets 4-1BB, OX40 and GITR.

Preferred combinations are combinations of a CD96 antibody of the invention and i) an immune check-point inhibitor or ii) an antibody against a tumor associated antigen. Exemplified combinations are herein described for Herceptin® and Erbitux®, wherein the combination with Herceptin® is preferred due to its additive, cooperative, or possibly synergistic effect. Other agents may also be useful to be combined.

The CD96-binding agents (e.g. monoclonal antibodies) of the invention may be used in vitro in immunoassays in which they can be utilized in liquid phase or bound to a solid phase carrier. In addition, the monoclonal antibodies in these immunoassays can be detectably labeled in various ways. Examples of types of immunoassays which can utilize monoclonal antibodies of the invention are flow cytometry, e.g. FACS, MACS, immunohistochemistry, competitive and non-competitive immunoassays in either direct or indirect formats; and the like. Detection of the antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The CD96-binding agents (e.g. monoclonal antibodies) of the invention can be bound to many different carriers and used to detect the presence of CD96-expressing cells. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

There are many different labels and methods of labeling known to those of ordinary skill in the art, which find use as tracers in therapeutic methods, for use in diagnostic methods, and the like. For diagnostic purposes a label may be covalently or non-covalently attached to an antibody of the invention or a fragment thereof, including fragments consisting or comprising of CDR sequences. Examples of the types of labels which can be used in the present invention include enzymes, radioisotopes, fluorescent compounds, colloidal metals, chemiluminescent compounds, and bio-luminescent compounds. Those of ordinary skill in the art will know of other suitable labels for binding to the CD96-binding agents (e.g. monoclonal antibodies) of the invention, or will be able to ascertain such, using routine experimentation. Furthermore, the binding of these labels to the CD96-binding agents (e.g. monoclonal antibodies) of the invention can be done using standard techniques common to those of ordinary skill in the art.

In some embodiments CD96-binding agent (e.g. monoclonal antibody or a fragment thereof) is attached to a nanoparticle, e.g. for use in imaging. Useful nanoparticles are those known in the art, for example including without limitation, Raman-silica-gold-nanoparticles (R-Si-Au-NP). The R-Si-Au-NPs consist of a Raman organic molecule, with a narrow-band spectral signature, adsorbed onto a gold core. Because the Raman organic molecule can be changed, each nanoparticle can carry its own signature, thereby allowing multiple nanoparticles to be independently detected simultaneously by multiplexing. The entire nanoparticle is encapsulated in a silica shell to hold the Raman organic molecule on the gold nanocore. Optional polyethylene glycol (PEG)-ylation of R-Si-Au-NPs increases their bioavailability and provides functional “handles” for attaching targeting moieties (see Thakor A. S., Luong R., Paulmurugan R., et al. et al., The fate and toxicity of raman-active silica-gold nanoparticles in mice, Sci. Transl. Med. 3(79): 79ra33 (2011); Jokerst IV., Miao Z., Zavaleta C., Cheng Z., and Gambhir S. S., Affibody-functionalized gold-silica nanoparticles for raman molecular imaging of the epidermal growth factor receptor, Small. 7(5):625-633 (2011); Gao J., Chen K, Miao Z., Ren G., Chen X, Gambhir S. S. and Cheng Z., Affibody-based nanoprobes for HER2-expressing cell and tumor imaging, Biomaterials 32(8):2141-2148 (2011); each herein specifically incorporated by reference).

For purposes of the invention, CD96 may be detected by the CD96-binding agents (e.g. monoclonal antibodies) of the invention when present in biological fluids and on tissues, in vivo or in vitro. Any sample containing a detectable amount of CD96 can be used. A sample can be a liquid such as urine, saliva, cerebrospinal fluid, blood, serum and the like, or a solid or semi-solid such as tissues, feces, and the like, or, alternatively, a solid tissue such as those commonly used in histological diagnosis.

Another labeling technique which may result in greater sensitivity consists of coupling the antibodies to low molecular weight haptens. These haptens can then be specifically detected by means of a second reaction. For example, it is common to use haptens such as biotin, which reacts with avidin, or dinitrophenol, pyridoxal, or fluorescein, which can react with specific anti-hapten antibodies.

As a matter of convenience, the CD96-binding agent (e.g. antibody) of the present invention can be provided in a kit, i.e., a packaged combination of reagents in predetermined amounts with instructions for performing the diagnostic assay. Where the CD96-binding agent (e.g. antibody) is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

Therapeutic formulations comprising one or more CD96-binding agents (e.g. antibodies) of the invention are prepared for storage by mixing the CD96-binding agent (e.g. antibody) having the desired degree of purity with optional physiologically acceptable carriers, excipients or stabilizers (Remington's Pharmaceutical Sciences, 16th edition, Osol, A. Ed. (1980)), in the form of lyophilized formulations or aqueous solutions. The composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the CD96-binding agent (e.g. antibody) to be administered will be governed by such considerations, and is the minimum amount necessary to prevent the disease.

The therapeutic dose may be at least about 0.01 mg per kg body weight, at least about 0.05 mg per kg body weight; at least about 0.1 mg per kg body weight, at least about 0.5 mg per kg body weight, at least about 1 mg per kg body weight, at least about 2.5 mg per kg body weight, at least about 5 mg per kg body weight, at least about 10 mg per kg body weight, and not more than about 100 mg per kg body weight with a preference of 0.1 to 20 mg per kg body weight. It will be understood by one of skill in the art that such guidelines will be adjusted for the molecular weight of the active agent, e.g. in the use of antibody fragments, or in the use of antibody conjugates. The dosage may also be varied for localized administration, e.g. intranasal, inhalation, etc., or for systemic administration, e.g., i.m., i.p., i.v., s.c., and the like.

The CD96-binding agent (e.g. antibody) need not be, but is optionally formulated with one or more agents that potentiate activity, or that otherwise increase the therapeutic effect. These are generally used in the same dosages and with administration routes as used hereinbefore or about from 1 to 99% of the heretofore employed dosages.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

The CD96-binding agent (e.g. antibody) is administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the CD96-binding agent (e.g. antibody) is suitably administered by pulse infusion, particularly with declining doses of the agent.

For the prevention or treatment of disease, the appropriate dosage of CD96-binding agent (e.g. antibody) will depend on the type of disease to be treated, as defined above, the severity and course of the disease, whether the agent is administered for preventive purposes, previous therapy, the patient's clinical history and response to the agent, and the discretion of the attending physician. The agent is suitably administered to the patient at one time or over a series of treatments.

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the CD96-binding agent (e.g. antibody). The label on, or associated with, the container indicates that the composition is used for treating the condition of choice. The article of manufacture may further comprise a second container comprising a pharmaceutically-acceptable buffer, such as phosphate-buffered saline, Ringer's solution and/or dextrose solution. It may further include other materials desirable from a commercial and user standpoint, including other buffers, diluents, filters, needles, syringes, and package inserts with instructions for use.

The invention now being fully described, it will be apparent to one of ordinary skill in the art that various changes and modifications can be made without departing from the spirit or scope of the invention.

TABLE 5
Overview of Seq Id numbers. When indicated,
X indicates ‘any’ amino acid, or
X can be absent.

| Seq | | | |
|---|---|---|---|
| ID | | | |
| NO | Name | | Sequence |
| 1 | BL006-4-20G5-20K5 | heavy chain, CDR1-Kabat | SYGMS |
| 2 | BL006-4-20G5-20K5 | heavy chain, CDR2-Kabat | TITHGGSYIYYPDSVKG |
| 3 | BL006-4-20G5-20K5 | heavy chain, CDR3-Kabat | HADYARFAY |
| 4 | BL006-4-20G5-20K5 | heavy chain, CDR1-IMGT | GFTFSSYG |
| 5 | BL006-4-20G5-20K5 | heavy chain, CDR2-IMGT | ITHGGSYI |
| 6 | BL006-4-20G5-20K5 | heavy chain, CDR3-IMGT | IRHADYARFAY |
| 7 | BL006-4-20G5-20K5 | light chain, CDR1-Kabat | RPSQDISNYLN |
| 8 | BL006-4-20G5-20K5 | light chain, CDR2-Kabat | YTSRLHS |
| 9 | BL006-4-20G5-20K5 | light chain, CDR3-Kabat | QQGNTLPWT |
| 10 | BL006-4-20G5-20K5 | light chain, CDR1-IMGT | QDISNY |
| 11 | BL006-4-20G5-20K5 | light chain, CDR2-IMGT | XYTS |
| 12 | BL006-4-20G5-20K5 | light chain, CDR3-IMGT | QQGNTLPWT |
| 13 | BL006-4-52G6-52K7 | heavy chain, CDR1-Kabat | SYWMH |
| 14 | BL006-4-52G6-52K7 | heavy chain, CDR2-Kabat | QIDPSDSYTNYNQKFKG |
| 15 | BL006-4-52G6-52K7 | heavy chain, CDR3-Kabat | GNYGPYLGNYFDY |
| 16 | BL006-4-52G6-52K7 | heavy chain, CDR1-IMGT | GYSFTSYW |
| 17 | BL006-4-52G6-52K7 | heavy chain, CDR2-IMGT | IDPSDSYT |
| 18 | BL006-4-52G6-52K7 | heavy chain, CDR3-IMGT | ASGNYGPYLGNYFDY |
| 19 | BL006-4-52G6-52K7 | light chain, CDR1-Kabat | KSSQSLLHSTNQKNYLA |
| 20 | BL006-4-52G6-52K7 | light chain, CDR2-Kabat | WASTRES |
| 21 | BL006-4-52G6-52K7 | light chain, CDR3-Kabat | QQYYNYPYT |
| 22 | BL006-4-52G6-52K7 | light chain, CDR1-IMGT | QSLLHSTNQKNY |
| 23 | BL006-4-52G6-52K7 | light chain, CDR2-IMGT | XWAS |
| 24 | BL006-4-52G6-52K7 | light chain, CDR3-IMGT | QQYYNYPYT |
| 25 | BL006-4-61G5-61K1 | heavy chain, CDR1-Kabat | NYWMN |
| 26 | BL006-4-61G5-61K1 | heavy chain, CDR2-Kabat | QIRFKSDNYATHYAESVKG |
| 27 | BL006-4-61G5-61K1 | heavy chain, CDR3-Kabat | XFAY |
| 28 | BL006-4-61G5-61K1 | heavy chain, CDR1-IMGT | GFTFSNYW |
| 29 | BL006-4-61G5-61K1 | heavy chain, CDR2-IMGT | IRFKSDNYAT |
| 30 | BL006-4-61G5-61K1 | heavy chain, CDR3-IMGT | TEFAY |
| 31 | BL006-4-61G5-61K1 | light chain, CDR1-Kabat | SASSRVNSRFLN |
| 32 | BL006-4-61G5-61K1 | light chain, CDR2-Kabat | GTSNLAS |
| 33 | BL006-4-61G5-61K1 | light chain, CDR3-Kabat | QQYQSDPPIT |
| 34 | BL006-4-61G5-61K1 | light chain, CDR1-IMGT | SRVNSRF |
| 35 | BL006-4-61G5-61K1 | light chain, CDR2-IMGT | XGTS |
| 36 | BL006-4-61G5-61K1 | light chain, CDR3-IMGT | QQYQSDPPIT |
| 37 | BL006-8-3G9-3K4 | heavy chain, CDR1-Kabat | DYEME |
| 38 | BL006-8-3G9-3K4 | heavy chain, CDR2-Kabat | AIDPETGGTAYNQKFKG |
| 39 | BL006-8-3G9-3K4 | heavy chain, CDR3-Kabat | EGDGSYGAY |
| 40 | BL006-8-3G9-3K4 | heavy chain, CDR1-IMGT | GYRFTDYE |
| 41 | BL006-8-3G9-3K4 | heavy chain, CDR2-IMGT | IDPETGGT |
| 42 | BL006-8-3G9-3K4 | heavy chain, CDR3-IMGT | TREGDGSYGAY |
| 43 | BL006-8-3G9-3K4 | light chain, CDR1-Kabat | RSSQSIVYSNGNTYLE |
| 44 | BL006-8-3G9-3K4 | light chain, CDR2-Kabat | KVSNRFS |
| 45 | BL006-8-3G9-3K4 | light chain, CDR3-Kabat | FQGSHAPWT |
| 46 | BL006-8-3G9-3K4 | light chain, CDR1-IMGT | QSIVYSNGNTY |
| 47 | BL006-8-3G9-3K4 | light chain, CDR2-IMGT | XKVS |
| 48 | BL006-8-3G9-3K4 | light chain, CDR3-IMGT | FQGSHAPWT |
| 49 | BL006-11-5G1-5K3 | heavy chain, CDR1-Kabat | SYAMS |
| 50 | BL006-11-5G1-5K3 | heavy chain, CDR2-Kabat | AINTDGASTYFPATVKD |
| 51 | BL006-11-5G1-5K3 | heavy chain, CDR3-Kabat | PLEPGSWFAY |
| 52 | BL006-11-5G1-5K3 | heavy chain, CDR1-IMGT | GFTFSSYA |
| 53 | BL006-11-5G1-5K3 | heavy chain, CDR2-IMGT | INTDGAST |
| 54 | BL006-11-5G1-5K3 | heavy chain, CDR3-IMGT | ARPLEPGSWFAY |
| 55 | BL006-11-5G1-5K3 | light chain, CDRl-Kabat | RSSQSIVHSNGNTYLE |
| 56 | BL006-11-5G1-5K3 | light chain, CDR2-Kabat | KVSNRFS |
| 57 | BL006-11-5G1-5K3 | light chain, CDR3-Kabat | FQGSHVPYT |
| 58 | BL006-11-5G1-5K3 | light chain, CDR1-IMGT | QSIVHSNGNTY |
| 59 | BL006-11-5G1-5K3 | light chain, CDR2-IMGT | XKVS |
| 60 | BL006-11-5G1-5K3 | light chain, CDR3-IMGT | FQGSHVPYT |
| 61 | BL006-19-14G3-14K1 | heavy chain, CDRl-Kabat | TFGMGVG |
| 62 | BL006-19-14G3-14K1 | heavy chain, CDR2-Kabat | HIWWDDDKFYNPALKS |
| 63 | BL006-19-14G3-14K1 | heavy chain, CDR3-Kabat | YYGSLSFDV |
| 64 | BL006-19-14G3-14K1 | heavy chain, CDR1-IMGT | GFSLDTFGMG |
| 65 | BL006-19-14G3-14K1 | heavy chain, CDR2-IMGT | IWWDDDK |
| 66 | BL006-19-14G3-14K1 | heavy chain, CDR3-IMGT | AHYYGSLSFDV |
| 67 | BL006-19-14G3-14K1 | light chain, CDRl-Kabat | KSSQSLLDSDGKTYLN |
| 68 | BL006-19-14G3-14K1 | light chain, CDR2-Kabat | LVSKLDS |
| 69 | BL006-19-14G3-14K1 | light chain, CDR3-Kabat | LQATHFPWT |
| 70 | BL006-19-14G3-14K1 | light chain, CDR1-IMGT | QSLLDSDGKTY |
| 71 | BL006-19-14G3-14K1 | light chain, CDR2-IMGT | XLVS |
| 72 | BL006-19-14G3-14K1 | light chain, CDR3-IMGT | LQATHFPWT |
| 73 | BL006-19-134G5-134K4 | heavy chain, CDRl-Kabat | TYWMH |
| 74 | BL006-19-134G5-134K4 | heavy chain, CDR2-Kabat | MIHPNSDTTLYNEKFRS |
| 75 | BL006-19-134G5-134K4 | heavy chain, CDR3-Kabat | TGTNFDY |
| 76 | BL006-19-134G5-134K4 | heavy chain, CDR1-IMGT | DYTFTTYW |
| 77 | BL006-19-134G5-134K4 | heavy chain, CDR2-IMGT | IHPNSDTT |
| 78 | BL006-19-134G5-134K4 | heavy chain, CDR3-IMGT | VVTGTNFDY |
| 79 | BL006-19-134G5-134K4 | light chain, CDRl-Kabat | SATSSVSYMY |
| 80 | BL006-19-134G5-134K4 | light chain, CDR2-Kabat | RTSNLAS |
| 81 | BL006-19-134G5-134K4 | light chain, CDR3-Kabat | QQWSSNPLT |
| 82 | BL006-19-134G5-134K4 | light chain, CDR1-IMGT | SSVSY |
| 83 | BL006-19-134G5-134K4 | light chain, CDR2-IMGT | XRTS |
| 84 | BL006-19-134G5-134K4 | light chain, CDR3-IMGT | QQWSSNPLT |
| 85 | BL006-19-183G3-183K4 | heavy chain, CDRl-Kabat | TYGMGVG |
| 86 | BL006-19-183G3-183K4 | heavy chain, CDR2-Kabat | NIWWTEDKYYNSALKS |
| 87 | BL006-19-183G3-183K4 | heavy chain, CDR3-Kabat | MGPEVYSAMDY |
| 88 | BL006-19-183G3-183K4 | heavy chain, CDR1-IMGT | GFSLTTYGMG |
| 89 | BL006-19-183G3-183K4 | heavy chain, CDR2-IMGT | IWWTEDK |
| 90 | BL006-19-183G3-183K4 | heavy chain, CDR3-IMGT | AQMGPEVYSAMDY |
| 91 | BL006-19-183G3-183K4 | light chain, CDRl-Kabat | KASQNVGTAVA |
| 92 | BL006-19-183G3-183K4 | light chain, CDR2-Kabat | SASNRNT |
| 93 | BL006-19-183G3-183K4 | light chain, CDR3-Kabat | HQYSRYPWT |
| 94 | BL006-19-183G3-183K4 | light chain, CDR1-IMGT | QNVGTA |
| 95 | BL006-19-183G3-183K4 | light chain, CDR2-IMGT | XSAS |
| 96 | BL006-19-183G3-183K4 | light chain, CDR3-IMGT | HQYSRYPWT |
| 97 | BL006-2-19G13-19K15 | heavy chain, CDRl-Kabat | SYWMH |
| 98 | BL006-2-19G13-19K15 | heavy chain, CDR2-Kabat | SIYPGSGSTNYDEKFKR |
| 99 | BL006-2-19G13-19K15 | heavy chain, CDR3-Kabat | EMAY |
| 100 | BL006-2-19G13-19K15 | heavy chain, CDR1-IMGT | GYKFTSYW |
| 101 | BL006-2-19G13-19K15 | heavy chain, CDR2-IMGT | IYPGSGST |
| 102 | BL006-2-19G13-19K15 | heavy chain, CDR3-IMGT | TREMAY |
| 103 | BL006-2-19G13-19K15 | light chain, CDRl-Kabat | RSSKSLLYKDGKTYLN |
| 104 | BL006-2-19G13-19K15 | light chain, CDR2-Kabat | WMSTRAS |
| 105 | BL006-2-19G13-19K15 | light chain, CDR3-Kabat | QQVVEYPFT |
| 106 | BL006-2-19G13-19K15 | light chain, CDR1-IMGT | KSLLYKDGKTY |
| 107 | BL006-2-19G13-19K15 | light chain, CDR2-IMGT | XWMS |
| 108 | BL006-2-19G13-19K15 | light chain, CDR3-IMGT | QQVVEYPFT |
| 109 | BL006-4-31G1-31K3 | heavy chain, CDR1-Kabat | DYYIN |
| 110 | BL006-4-31G1-31K3 | heavy chain, CDR2-Kabat | WIFPGSGSSFYSEIFKG |
| 111 | BL006-4-31G1-31K3 | heavy chain, CDR3-Kabat | RNYYGYGAYAMDY |
| 112 | BL006-4-31G1-31K3 | heavy chain, CDR1-IMGT | GFTFTDYY |
| 113 | BL006-4-31G1-31K3 | heavy chain, CDR2-IMGT | IFPGSGSS |
| 114 | BL006-4-31G1-31K3 | heavy chain, CDR3-IMGT | ARRNYYGYGAYAMDY |
| 115 | BL006-4-31G1-31K3 | light chain, CDR1-Kabat | RASQSINFWLS |
| 116 | BL006-4-31G1-31K3 | light chain, CDR2-Kabat | KASNLHT |
| 117 | BL006-4-31G1-31K3 | light chain, CDR3-Kabat | LQGQSYPLT |
| 118 | BL006-4-31G1-31K3 | light chain, CDR1-IMGT | QSINFW |
| 119 | BL006-4-31G1-31K3 | light chain, CDR2-IMGT | XYTS |
| 120 | BL006-4-31G1-31K3 | light chain, CDR3-IMGT | LQGQSYPLT |
| 121 | BL006-9-1G1-1K1 | heavy chain, CDR1-Kabat | SYAMS |
| 122 | BL006-9-1G1-1K1 | heavy chain, CDR2-Kabat | AIQSNGGSTFYPDTVKD |
| 123 | BL006-9-1G1-1K1 | heavy chain, CDR3-Kabat | GPFDY |
| 124 | BL006-9-1G1-1K1 | heavy chain, CDR1-IMGT | GFTFSSYA |
| 125 | BL006-9-1G1-1K1 | heavy chain, CDR2-IMGT | IQSNGGST |
| 126 | BL006-9-1G1-1K1 | heavy chain, CDR3-IMGT | VRGPFDY |
| 127 | BL006-9-1G1-1K1 | light chain, CDR1-Kabat | SASSSVNYIH |
| 128 | BL006-9-1G1-1K1 | light chain, CDR2-Kabat | DTSKLTP |
| 129 | BL006-9-1G1-1K1 | light chain, CDR3-Kabat | HQWRSYPPT |
| 130 | BL006-9-1G1-1K1 | light chain, CDR1-IMGT | SSVNY |
| 131 | BL006-9-1G1-1K1 | light chain, CDR2-IMGT | XDTS |
| 132 | BL006-9-1G1-1K1 | light chain, CDR3-IMGT | HQWRSYPPT |
| 133 | BL006-9-15G1-15K5 | heavy chain, CDR1-Kabat | SYNMH |
| 134 | BL006-9-15G1-15K5 | heavy chain, CDR2-Kabat | AVIPGNGDSFYNQKFKG |
| 135 | BL006-9-15G1-15K5 | heavy chain, CDR3-Kabat | SSSGCIAY |
| 136 | BL006-9-15G1-15K5 | heavy chain, CDR1-IMGT | GYTFTSYN |
| 137 | BL006-9-15G1-15K5 | heavy chain, CDR2-IMGT | VIPGNGDS |
| 138 | BL006-9-15G1-15K5 | heavy chain, CDR3-IMGT | RSSSGCIAY |
| 139 | BL006-9-15G1-15K5 | light chain, CDR1-Kabat | RVSGSVDYDGDSYMN |
| 140 | BL006-9-15G1-15K5 | light chain, CDR2-Kabat | AASNLES |
| 141 | BL006-9-15G1-15K5 | light chain, CDR3-Kabat | LQSNEAPWT |
| 142 | BL006-9-15G1-15K5 | light chain, CDR1-IMGT | GSVDYDGDSY |
| 143 | BL006-9-15G1-15K5 | light chain, CDR2-IMGT | XAAS |
| 144 | BL006-9-15G1-15K5 | light chain, CDR3-IMGT | LQSNEAPWT |
| 145 | BL006-19-21G1-21K1 | heavy chain, CDR1-Kabat | SYWIT |
| 146 | BL006-19-21G1-21K1 | heavy chain, CDR2-Kabat | DIYPGSGHTNYNEKFKS |
| 147 | BL006-19-21G1-21K1 | heavy chain, CDR3-Kabat | PTVEAMDY |
| 148 | BL006-19-21G1-21K1 | heavy chain, CDR1-IMGT | GYTFISYW |
| 149 | BL006-19-21G1-21K1 | heavy chain, CDR2-IMGT | IYPGSGHT |
| 150 | BL006-19-21G1-21K1 | heavy chain, CDR3-IMGT | ARPTVEAMDY |
| 151 | BL006-19-21G1-21K1 | light chain, CDR1-Kabat | SASSSVNYMH |
| 152 | BL006-19-21G1-21K1 | light chain, CDR2-Kabat | DTSKLAS |
| 153 | BL006-19-21G1-21K1 | light chain, CDR3-Kabat | QQWSNNPLT |
| 154 | BL006-19-21G1-21K1 | light chain, CDR1-IMGT | SSVNY |
| 155 | BL006-19-21G1-21K1 | light chain, CDR2-IMGT | XDTS |
| 156 | BL006-19-21G1-21K1 | light chain, CDR3-IMGT | QQWSNNPLT |
| 157 | BL006-19-29G5-29K1 | heavy chain, CDR1-Kabat | TFGMGVS |
| 158 | BL006-19-29G5-29K1 | heavy chain, CDR2-Kabat | HIFWDDDKRYNPSLKS |
| 159 | BL006-19-29G5-29K1 | heavy chain, CDR3-Kabat | GSNWFAY |
| 160 | BL006-19-29G5-29K1 | heavy chain, CDR1-IMGT | GFSLSTFGMG |
| 161 | BL006-19-29G5-29K1 | heavy chain, CDR2-IMGT | IFWDDDK |
| 162 | BL006-19-29G5-29K1 | heavy chain, CDR3-IMGT | TRGSNWFAY |
| 163 | BL006-19-29G5-29K1 | light chain, CDR1-Kabat | RASESVDSYGNSFMH |
| 164 | BL006-19-29G5-29K1 | light chain, CDR2-Kabat | RASNLES |
| 165 | BL006-19-29G5-29K1 | light chain, CDR3-Kabat | QQSNEDPLT |
| 166 | BL006-19-29G5-29K1 | light chain, CDR1-IMGT | ESVDSYGNSF |
| 167 | BL006-19-29G5-29K1 | light chain, CDR2-IMGT | XRAS |
| 168 | BL006-19-29G5-29K1 | light chain, CDR3-IMGT | QQSNEDPLT |
| 169 | BL006-19-55G10-55K7 | heavy chain, CDR1-Kabat | DNWID |
| 170 | BL006-19-55G10-55K7 | heavy chain, CDR2-Kabat | NIFPGGNYTNYNEKFKG |
| 171 | BL006-19-55G10-55K7 | heavy chain, CDR3-Kabat | AVADSQFAY |
| 172 | BL006-19-55G10-55K7 | heavy chain, CDR1-IMGT | GYTFTDNW |
| 173 | BL006-19-55G10-55K7 | heavy chain, CDR2-IMGT | IFPGGNYT |
| 174 | BL006-19-55G10-55K7 | heavy chain, CDR3-IMGT | VRAVADSQFAY |
| 175 | BL006-19-55G10-55K7 | light chain, CDR1-Kabat | SASSRVTYMY |
| 176 | BL006-19-55G10-55K7 | light chain, CDR2-Kabat | RTSNLAS |
| 177 | BL006-19-55G10-55K7 | light chain, CDR3-Kabat | QQWSSNPLT |
| 178 | BL006-19-55G10-55K7 | light chain, CDR1-IMGT | SRVTY |
| 179 | BL006-19-55G10-55K7 | light chain, CDR2-IMGT | XRTS |
| 180 | BL006-19-55G10-55K7 | light chain, CDR3-IMGT | QQWSSNPLT |
| 181 | BL006-19-190G4-190K6 | heavy chain, CDR1-Kabat | NYGMS |
| 182 | BL006-19-190G4-190K6 | heavy chain, CDR2-Kabat | TINSNGGSTYYPDSVKG |
| 183 | BL006-19-190G4-190K6 | heavy chain, CDR3-Kabat | RGAWYFDV |
| 184 | BL006-19-190G4-190K6 | heavy chain, CDR1-IMGT | GFTFSNYG |
| 185 | BL006-19-190G4-190K6 | heavy chain, CDR2-IMGT | INSNGGST |
| 186 | BL006-19-190G4-190K6 | heavy chain, CDR3-IMGT | KRGAWYFDV |
| 187 | BL006-19-190G4-190K6 | light chain, CDR1-Kabat | KASEDIYNRLA |
| 188 | BL006-19-190G4-190K6 | light chain, CDR2-Kabat | SATSLET |
| 189 | BL006-19-190G4-190K6 | light chain, CDR3-Kabat | QQYWTTPTWT |
| 190 | BL006-19-190G4-190K6 | light chain, CDR1-IMGT | EDIYNR |
| 191 | BL006-19-190G4-190K6 | light chain, CDR2-IMGT | XSAT |
| 192 | BL006-19-190G4-190K6 | light chain, CDR3-IMGT | QQYWTTPTWT |
| 193 | BL006-19-352G9-352K8 | heavy chain, CDR1-Kabat | SYWMH |
| 194 | BL006-19-352G9-352K8 | heavy chain, CDR2-Kabat | MIHPNGDITDYNEKFKS |
| 195 | BL006-19-352G9-352K8 | heavy chain, CDR3-Kabat | ASYYGSLY |
| 196 | BL006-19-352G9-352K8 | heavy chain, CDR1-IMGT | GYTFTSYW |
| 197 | BL006-19-352G9-352K8 | heavy chain, CDR2-IMGT | IHPNGDIT |
| 198 | BL006-19-352G9-352K8 | heavy chain, CDR3-IMGT | ARASYYGSLY |
| 199 | BL006-19-352G9-352K8 | light chain, CDR1-Kabat | KASQNVGIAVA |
| 200 | BL006-19-352G9-352K8 | light chain, CDR2-Kabat | SASTRYT |
| 201 | BL006-19-352G9-352K8 | light chain, CDR3-Kabat | QQYGSSPLT |
| 202 | BL006-19-352G9-352K8 | light chain, CDR1-IMGT | QNVGIA |
| 203 | BL006-19-352G9-352K8 | light chain, CDR2-IMGT | XSAS |
| 204 | BL006-19-352G9-352K8 | light chain, CDR3-IMGT | QQYGSSPLT |
| 205 | BL006-19-363G1-363K1 | heavy chain, CDR1-Kabat | TYWIH |
| 206 | BL006-19-363G1-363K1 | heavy chain, CDR2-Kabat | MILPNSGTTIYNEKFRS |
| 207 | BL006-19-363G1-363K1 | heavy chain, CDR3-Kabat | TGTNFDY |
| 208 | BL006-19-363G1-363K1 | heavy chain, CDR1-IMGT | GYTFTTYW |
| 209 | BL006-19-363G1-363K1 | heavy chain, CDR2-IMGT | ILPNSGTT |
| 210 | BL006-19-363G1-363K1 | heavy chain, CDR3-IMGT | AVTGTNFDY |
| 211 | BL006-19-363G1-363K1 | light chain, CDR1-Kabat | SASSSVSYMY |
| 212 | BL006-19-363G1-363K1 | light chain, CDR2-Kabat | RTSNLAS |
| 213 | BL006-19-363G1-363K1 | light chain, CDR3-Kabat | QQWSSNPLT |
| 214 | BL006-19-363G1-363K1 | light chain, CDR1-IMGT | SSVSY |
| 215 | BL006-19-363G1-363K1 | light chain, CDR2-IMGT | XRTS |
| 216 | BL006-19-363G1-363K1 | light chain, CDR3-IMGT | QQWSSNPLT |
| 217 | BL006-19-370G9-370K16 | heavy chain, CDR1-Kabat | NYWIG |
| 218 | BL006-19-370G9-370K16 | heavy chain, CDR2-Kabat | DIYPGGGYTNYNEKFKG |
| 219 | BL006-19-370G9-370K16 | heavy chain, CDR3-Kabat | SGYYDGSPLAMDY |
| 220 | BL006-19-370G9-370K16 | heavy chain, CDR1-IMGT | GYTFTNYW |
| 221 | BL006-19-370G9-370K16 | heavy chain, CDR2-IMGT | IYPGGGYT |
| 222 | BL006-19-370G9-370K16 | heavy chain, CDR3-IMGT | ARSGYYDGSPLAMDY |
| 223 | BL006-19-370G9-370K16 | light chain, CDR1-Kabat | SASSSVSYMH |
| 224 | BL006-19-370G9-370K16 | light chain, CDR2-Kabat | DTSKLAS |
| 225 | BL006-19-370G9-370K16 | light chain, CDR3-Kabat | QQWSSNPPT |
| 226 | BL006-19-370G9-370K16 | light chain, CDR1-IMGT | SSVSY |
| 227 | BL006-19-370G9-370K16 | light chain, CDR2-IMGT | XDTS |
| 228 | BL006-19-370G9-370K16 | light chain, CDR3-IMGT | QQWSSNPPT |
| 229 | BL006-4-20G5-20K5 | variable region heavy chain | EVQLAESGGDLVTPGGSLKLSCAASGFTFSSYGM |
| | | | SWVRQTPDKRLEWVATITHGGSYIYYPDSVKGR |
| | | | FTLSRDNAKNTLYLQMSSLKSEDTAMYYCIRHA |
| | | | DYARFAYWGQGTLVTVSA |
| 230 | BL006-4-20G5-20K5 | variable region light chain | DIQMTQTTSSLSASLGDRVTISCRPSQDISNYLNW |
| | | | YQQKPDGTVKLLIYYTSRLHSGVPSRFSGSGSGT |
| | | | DYSLTISNLEQEDIATYFCQQGNTLPWTFGGGTK |
| | | | LEK |
| 231 | BL006-4-52G6-52K7 | variable region heavy chain | QVQLQQPGAELVKPGASVKLSCKASGYSFTSYW |
| | | | MHWVKQRPGQGLEWIGQIDPSDSYTNYNQKFK |
| | | | GKATLTVDKSSSTAYMQLSSLTSEDSAVYYCAS |
| | | | GNYGPYLGNYFDYWGQGTTLTVPS |
| 232 | BL006-4-52G6-52K7 | variable region light chain | DIVMSQSPSSLVVSVGEKVTMSCKSSQSLLHSTN |
| | | | QKNYLAWYQQKPGQSPKLLIYWASTRESGVPDR |
| | | | FTGSGSGTDFTLTISSVKAEDLVVYYCQQYYNYP |
| | | | YTFGGGTKLEIK |
| 233 | BL006-4-61G5-61K1 | variable region heavy chain | EVKLEESGGGLVQPGGSMKLSCVASGFTFSNYW |
| | | | MNWVRQSPEKGLEWVAQIRFKSDNYATHYAES |
| | | | VKGRFTISRDDSKSSVYLQMHNLRAEDTGIYYCT |
| | | | EFAYWGQGTLVTVSA |
| 234 | BL006-4-61G5-61K1 | variable region light chain | QILLTQSPAIMSASPGEKVTMTCSASSRVNSRFLN |
| | | | WYQQKSGVSPKLWIYGTSNLASGVPARFSGSGS |
| | | | GTSYSLTISSVEAEDVATYYCQQYQSDPPITFGAG |
| | | | TRLELI |
| 235 | BL006-8-3G9-3K4 | variable region heavy chain | QVQLQQSGAELVRPGASVKLSCKASGYRFTDYE |
| | | | MEWVKQTPVHGLEWIGAIDPETGGTAYNQKFKG |
| | | | KATVTADKSSRTAYMELRSLTSEDSAVYYCTRE |
| | | | GDGSYGAYWGQGTLVTVSA |
| 236 | BL006-8-3G9-3K4 | variable region light chain | DVMMTQTPLSLPVSLGDQASISCRSSQSIVYSNG |
| | | | NTYLEWYLQKPGQSPKLLMYKVSNRFSGVPDRF |
| | | | SGSGSGTDFTLKISRVEAEDLGVYYCFQGSHAPW |
| | | | TFGGGTKLEIK |
| 237 | BL006-11-5G1-5K3 | variable region heavy chain | EVQLVESGGGLVKPGGSLKLSCAASGFTFSSYAM |
| | | | SWVRQTPEKRLEWVAAINTDGASTYFPATVKDR |
| | | | FTISRDNAKNTLYLQMSSLRSEDTALYYCARPLE |
| | | | PGSWFAYWGQGTLVTVSA |
| 238 | BL006-11-5G1-5K3 | variable region light chain | DVLMTQTPLSLPVSLGDQASISCRSSQSIVHSNGN |
| | | | TYLEWYLQKPGQSPKVLIYKVSNRFSGVPDRISG |
| | | | SGSGTDFTLKISRVEAGDLGVYYCFQGSHVPYTF |
| | | | GGGTKLEIK |
| 239 | BL006-19-14G3-14K1 | variable region heavy chain | QVTLKESGPGILQPSQTLSLTCSFSGFSLDTFGM |
| | | | GVGWIRQSSGKGLEWLAHIWWDDDKFYNPALKS |
| | | | RLTVSKDTSKNQVFLKIANVDTADTATYYCAHY |
| | | | YGSLSFDVWGTGTTVTVSS |
| 240 | BL006-19-14G3-14K1 | variable region light chain | DVVMTQTPLTLSVTIGQPASISCKSSQSLLDSDGK |
| | | | TYLNWLLQRPGESPKLLIYLVSKLDSGVPDRFTG |
| | | | SGSGTDFTLKISRVEAEDLGVYYCLQATHFPWTF |
| | | | GGGTKLEIK |
| 241 | BL006-19-134G5-134K4 | variable region heavy chain | QVQLQQPGAELVKPGASVKLSCKASDYTFTTYW |
| | | | MHWVKQRPGQGLEWIGMIHPNSDTTLYNEKFRS |
| | | | QATLTVDKSSSTAYIQLSRLTSEDSAVYYCWTG |
| | | | TNFDYWGQGTTLAVSS |
| 242 | BL006-19-134G5-134K4 | variable region light chain | QVVLTQSPAIMSASPGEKVTMTCSATSSVSYMY |
| | | | WYQQKPGSSPKPWIYRTSNLASGVPVRFSGSGSG |
| | | | TSYSLTISSMEAEDAATYYCQQWSSNPLTFGGGT |
| | | | KLELK |
| 243 | BL006-19-183G3-183K4 | variable region heavy chain | QVTLNESGPGILQPSQTLSLTCSFSGFSLTTYGMG |
| | | | VGWIRQPSGKSLEWLANIWWTEDKYYNSALKSR |
| | | | LTISTDTSHNQVFLKISSVDTEDTATYYCAQMGP |
| | | | EVYSAMDYWGQGTSVTVSS |
| 244 | BL006-19-183G3-183K4 | variable region light chain | DIVMTPSQKFMSTTVGDRVSITCKASQNVGTAV |
| | | | AWYQQKPGHSPKLLIYSASNRNTGVPDRFTGSGS |
| | | | GTDFTLTISNMQSEDLADYFCHQYSRYPWTFGG |
| | | | GTKLEIK |
| 245 | BL006-2-19G13-19K15 | variable region heavy chain | QVQLQQPGSELVRPGASVKLSCKASGYKFTSYW |
| | | | MHWVKQRPGQGLEWIGSIYPGSGSTNYDEKFKR |
| | | | KATLTVDTFSSTASMQLSSLTSEDSAVYYCTREM |
| | | | AYWGQGTLVTVSA |
| 246 | BL006-2-19G13-19K15 | variable region light chain | DIVITQDELSNPVTSGESVSISCRSSKSLLYKDGKT |
| | | | YLNWFLQRPGQSPQLLVYWMSTRASGVSDRFSG |
| | | | SGSGTDFTLEISRVKAEDVGVYYCQQVVEYPFTF |
| | | | GTGTKLGIK |
| 247 | BL006-4-31G1-31K3 | variable region heavy chain | QVQLQQSGPELVKPGASVKISCKTSGFIFIDYYI |
| | | | NWVKQRPGQGLEWIGWIFPGSGSSFYSEIFKGQA |
| | | | TLTVDKSSSTAYMLLSSLTSEDSAVYFCARRNYY |
| | | | GYGAYAMDYWGQGTSVTVSS |
| 248 | BL006-4-31G1-31K3 | variable region light chain | DIQMNQSPSSLSASLGDTITITCRASQSINFWLSW |
| | | | YQQKPGNIPKLLIYKASNLHTGVPSRFSGSGSGTD |
| | | | FTLTISSLQPEDIATYYCLQGQSYPLTFGGGTKLEI |
| | | | K |
| 249 | BL006-9-1G1-1K1 | variable region heavy chain | EVKLVESGGGLVKPGGSLKLSCAASGFTFSSYAM |
| | | | SWVRQTPERGLEWVAAIQSNGGSTFYPDTVKDR |
| | | | FTISRDNAKNTLYLQMSSLRSEDTALYYCVRGPF |
| | | | DYWGQGTTLTVSS |
| 250 | BL006-9-1G1-1K1 | variable region light chain | QIVLTQSPAIMSASPGQKVTIPCSASSSVNYIHWF |
| | | | QQKLGSSPKLWIYDTSKLTPGVPARFSGSGSGTS |
| | | | YSLTISSMEAEDAASYFCHQWRSYPPTFGGGTKL |
| | | | EIK |
| 251 | BL006-9-15G1-15K5 | variable region heavy chain | QAYLQQSGAELVRPGASVKMSCKASGYTFTSYN |
| | | | MHWVKQTPRQGLEWIGAVIPGNGDSFYNQKFK |
| | | | GKATLTVDKSSSTAYMQLSSLTSEDSAVYFCGRS |
| | | | SSGCIAYWGQGTLVTVSA |
| 252 | BL006-9-15G1-15K5 | variable region light chain | DIVLTQSPASLAVSLGQRATISCRVSGSVDYDGD |
| | | | SYMNWYQQKPGQPPKLLIYAASNLESGIPARFSG |
| | | | SGSETDFTLNIHPVEEEDAATYYCLQSNEAPWTF |
| | | | GGGTKVEIK |
| 253 | BL006-19-21G1-21K1 | variable region heavy chain | QVQLQQPGAELVKPGASVKMSCKASGYTFISYW |
| | | | ITWVKQRPGQGLEWIGDIYPGSGHTNYNEKFKSK |
| | | | ATLTVDTSSSTAYMQLSSLTSEDSAVYYCARPTV |
| | | | EAMDYWGQGTSVTVSS |
| 254 | BL006-19-21G1-21K1 | variable region light chain | QIVLTQSPAIMSASPGEKVTMTCSASSSVNYMHW |
| | | | YQQRSGTSPKRWIYDTSKLASGVPARFSGSGSGT |
| | | | SYSLTISSMEAEDAATYYCQQWSNNPLTFGAGT |
| | | | KLELK |
| 255 | BL006-19-29G5-29K1 | variable region heavy chain | QVTLKESGPGILQSSQTLSLTCSFSGFSLSTFGMG |
| | | | VSWIRKPSGKGLEWLAHIFWDDDKRYNPSLKSR |
| | | | LTISKDTSSNQVFLMINSMDIADTATYYCTRGSN |
| | | | WFAYWGQGTLVTVSA |
| 256 | BL006-19-29G5-29K1 | variable region light chain | DIVLTQSPASLAVSLGQRATISCRASESVDSYGNS |
| | | | FMHWYQQKPGQPPKLLIYRASNLESGIPARFSGS |
| | | | GSRTDFTLTINPVEADDVATYYCQQSNEDPLTFG |
| | | | AGTKLELK |
| 257 | BL006-19-55G10-55K7 | variable region heavy chain | QVQLQQSGAEVVRPGTSVKMSCKAAGYTFTDN |
| | | | WIDWVKQRPGHGLDWIGNIFPGGNYTNYNEKFK |
| | | | GKATLTADTSSSTAYMQLINLTSEDSAIYYCVRA |
| | | | VADSQFAYWGQGTLVTVSA |
| 258 | BL006-19-55G10-55K7 | variable region light chain | QIVLTQSPAIMSVSPGEKVTMTCSASSRVTYMYW |
| | | | YQQKPGSSPKPWIYRTSNLASGVPVRFSGSGSGT |
| | | | SYSLTISSMEAEDAATYYCQQWSSNPLTFGAGTK |
| | | | LELK |
| 259 | BL006-19-190G4-190K6 | variable region heavy chain | EVQLVESGGGLVQPGGSLKLSCAASGFTFSNYG |
| | | | MSWVRQTPDKRLELVATINSNGGSTYYPDSVKG |
| | | | RFTISRDNAKNTLYLQMSSLKSEDTAMYYCTKR |
| | | | GAWYFDVWGTGTTVTVSS |
| 260 | BL006-19-190G4-190K6 | variable region light chain | DIQMTQSSSSFSVSLGDRVTITCKASEDIYNRLAW |
| | | | YQQKPGNAPRLLISSATSLETGVSSRFSGSGSGKD |
| | | | YTLSITSLQTEDVATYYCQQYWTTPTWTFGGGT |
| | | | KLEIK |
| 261 | BL006-19-352G9-352K8 | variable region heavy chain | QVQLQQPGAELVKPGASVKLSCKASGYTFTSYW |
| | | | MHWVKQRPGQGLEWIGMIHPNGDITDYNEKFKS |
| | | | KATLAVDESSSTAFMQLNSLTSEDSAFYYCARAS |
| | | | YYGSLYWGQGTTLTVSS |
| 262 | BL006-19-352G9-352K8 | variable region light chain | DIVMTQSQKFMSTSVGDRVSVTCKASQNVGIAV |
| | | | AWYQKKPGQSPKTLIYSASTRYTGVPDRFTGSGS |
| | | | GTDFTLTINDVQSEDLADYFCQQYGSSPLTFGAG |
| | | | TKLELK |
| 263 | BL006-19-363G1-363K1 | variable region heavy chain | QVQLQQPGAELVKPGASVKLSCKASGYTFTTYW |
| | | | IHWVKQRPGQGLEWIGMILPNSGTTIYNEKFRSQ |
| | | | ATLTVDKSSSAAYMQLSRLTSEDSAVYYCAVTG |
| | | | TNFDYWGQGTTLTVSS |
| 264 | BL006-19-363G1-363K1 | variable region light chain | QIVLTQSPAIMSASPGEKVTMTCSASSSVSYMYW |
| | | | YQQKPGSSPKPWIYRTSNLASGVPVRFSGSGFGT |
| | | | SYSLTISNMEAEDAATYYCQQWSSNPLTFGTGTK |
| | | | LELK |
| 265 | BL006-19-370G9-370K16 | variable region heavy chain | QVQLQQSGAELGRPGTSVKMSCKAAGYTFTNY |
| | | | WIGWVKQRPGHGLEWIGDIYPGGGYTNYNEKFK |
| | | | GKATLTADTSSSTAYIQLSSLTSEDSAIYYCARSG |
| | | | YYDGSPLAMDYWGQGTSVTVS |
| 266 | BL006-19-370G9-370K16 | variable region light chain | QIVLTQSPTIMSASPGEKVTMTCSASSSVSYMHW |
| | | | YQQKSGTSPKRWIYDTSKLASGVPARFSGSGSGT |
| | | | SYSLTISSMEAEDAATYYCQQWSSNPPTFGSGTK |
| | | | LEK |
| 267 | Human CD 96 | | MEKKWKYCAVYYIIQIHFVKGVWEKTVNTEENV |
| | variant 1 (long isoform) | | YATLGSDVNLTCQTQTVGFFVQMQWSKVTNKID |
| | | | LIAVYHPQYGFYCAYGRPCESLVTFTETPENGSK |
| | | | WTLHLRNMSCSVSGRYECMLVLYPEGIQTKIYN |
| | | | LLIQTHVTADEWNSNHTIEIEINQTLEIPCFQNSSS |
| | | | KISSEFTYAWSVENSSTDSWVLLSKGIKEDNGTQ |
| | | | ETLISQNHLISNSTLLKDRVKLGTDYRLHLSPVQI |
| | | | FDDGRKFSCHIRVGPNKILRSSTTVKVFAKPEIPVI |
| | | | VENNSTDVLVERRFTCLLKNVFPKANITWFIDGS |
| | | | FLHDEKEGIYITNEERKGKDGFLELKSVLTRVHS |
| | | | NKPAQSDNLTIWCMALSPVPGNKVWNISSEKITF |
| | | | LLGSEISSTDPPLSVTESTLDTQPSPASSVSPARYP |
| | | | ATSSVTLVDVSALRPNTTPQPSNSSMTTRGFNYP |
| | | | WTSSGTDTKKSVSRIPSETYSSSPSGAGSTLHDNV |
| | | | FTSTARAFSEVPTTANGSTKTNHVHITGIVVNKPK |
| | | | DGMSWPVIVAALLFCCMILFGLGVRKWCQYQKE |
| | | | IMERPPPFKPPPPPIKYTCIQEPNESDLPYHEMETL |
| 268 | Human CD 96, variant 2 | | MEKKWKYCAVYYIIQIHFVKGVWEKTVNTEENV |
| | (long isoform) | | YATLGSDVNLTCQTQTVGFFVQMQWSKVTNKID |
| | | | LIAVYHPQYGFYCAYGRPCESLVTFTETPENGSK |
| | | | WTLHLRNMSCSVSGRYECMLVLYPEGIQTKIYN |
| | | | LLIQTHVTADEWNSNHTIEIEINQTLEIPCFQNSSS |
| | | | KISSEFTYAWSVEDNGTQETLISQNHLISNSTLLK |
| | | | DRVKLGTDYRLHLSPVQIFDDGRKFSCHIRVGPN |
| | | | KILRSSTTVKVFAKPEIPVIVENNSTDVLVERRFT |
| | | | CLLKNVFPKANITWFIDGSFLHDEKEGIYITNEER |
| | | | KGKDGFLELKSVLTRVHSNKPAQSDNLTIWCMA |
| | | | LSPVPGNKVWNISSEKITFLLGSEISSTDPPLSVTE |
| | | | STLDTQPSPASSVSPARYPATSSVTLVDVSALRPN |
| | | | TTPQPSNSSMTTRGFNYPWTSSGTDTKKSVSRIPS |
| | | | ETYSSSPSGAGSTLHDNVFTSTARAFSEVPTTANG |
| | | | STKTNHVHITGIVVNKPKDGMSWPVIVAALLFCC |
| | | | MILFGLGVRKWCQYQKEIMERPPPFKPPPPPIKYT |
| | | | CIQEPNESDLPYHEMETL |
| 269 | Human CD 96, Ig-like | residues 38-125 of SEQ ID | GSDVNLTCQTQTVGFFVQMQWSKVTNKIDLIAV |
| | (V-type 1) domain 1 | NO: 267 | YHPQYGFYCAYGRPCESLVTFTETPENGSKWTL |
| | | | HLRNMSCSVSGRYECMLVLYPE |
| 270 | Human CD 96, IG-like | residues 156-238 of SEQ ID | NQTLEIPCFQNSSSKISSEFTYAWSVENSSTDSWV |
| | (V-type 2) domain 2 | NOD: 267 | LLSKGIKEDNGTQETLISQNHLISNSTLLKDRVKL |
| | | | GTDYRLHLSPVQI |
| 271 | Human CD96, Ig-like | residues 269-375 of SEQ ID | PEIPVIVENNSTDVLVERRFTCLLKNVFPKANITW |
| | (C2 type) domain 3 | NO: 267 | FIDGSFLHDEKEGIYITNEERKGKDGFLELKSVLT |
| | | | RVHSNKPAQSDNLTIWCMALSPVPGNKVWNISS |
| | | | EKIT |
| 272 | Rhesus CD96, | from NCBI accession | MEKKWKYCAVYYIIQIHFVKGVWGKPLNTEENI |
| | short isoform | number XP_00548247 | YATLGSDVNLTCQTQAKGFLVQMQWSKVTDKA |
| | | | DLIALYHPQYGFHCAYGSPCESLVTFTQTPENGS |
| | | | KWTLHLRNMSSSVSGRYECMLTLYPEGMQTKIY |
| | | | NLLIQTHVTPDEWKSNHTIEIEINQTLEIPCFQNSS |
| | | | SEISSEFTYAWLVEDNGTQQTLISQDHLISSSTLLK |
| | | | DRVKVGTDYRLHLSPVQIFDDGRKFSCHIRVGPD |
| | | | KILRSSTTIKVFAKPEIPMIVENNSTDVLVERTFTC |
| | | | LLTNVFPKANIIWFIDGSFLHDEKEGIYITNEERKG |
| | | | KDGFLELKSVLTRVHSDKPAQSDNLTIWCMALSP |
| | | | VPGNKVWNISSEKITFLLGSEMSTTDPPPSVTEST |
| | | | LDTQPSPASSVSPTRYPATSSVTLADVSALRPNTT |
| | | | PQSSSSSVTTQDFNYPWTSSGTDAKKSFSQIPSET |
| | | | YSLSPSGAGSTLHDNVFTSTTRALSEVPTTANGST |
| | | | KTNHVHITGIVVSKPKDGMSWPVIVAALLFCCMI |
| | | | LFVLGVRKWCQYQKEIMERPPPFKPPPPPIKYTCI |
| | | | QEPNESDPPYHEMETL |
| 273 | Human CD69, Ig-like | residues 156-222 of SEQ ID | NQTLEIPCFQNSSSKISSEFTYAWSVEDNGTQETLI |
| | (V-type 2) domain 2 | NO: 268 | SQNHLISNSTLLKDRVKLGTDYRLHLSPVQI |
| 274 | Human CD96, | residues 370-502 of SEQ ID | SSEKITFLLGSEISSTDPPLSVTESTLDTQPSPASSV |
| | Pro/Ser/Thr- | NO: 267 | SPARYPATSSVTLVDVSALRPNTTPQPSNSSMTTR |
| | rich domain 4 | | GFNYPWTSSGTDTKKSVSRIPSETYSSSPSGAGST |
| | | | LHDNVFTSTARAFSEVPTTANGSTKT |

### EXAMPLES

1. Characterization of the Human CD96

CD96 Expression on T, NK and Other Hematopoietic Cell Populations

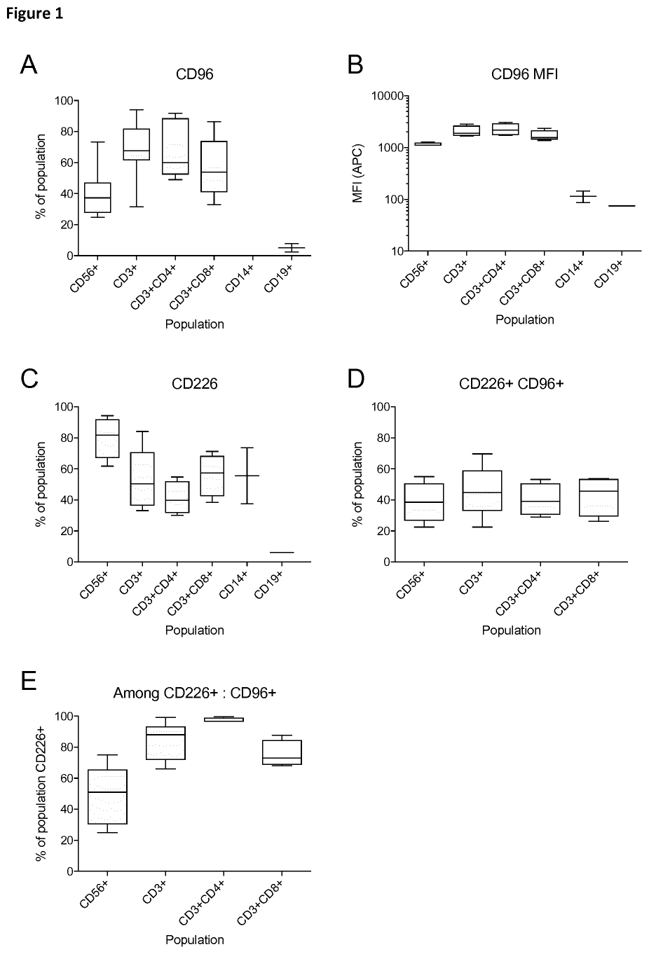

Expression of the human CD96 on the different populations of human PBMCs has been tested by using anti-CD96 clone 628211 (R&D Biotech) and NK92.39 (Fuchs, 2004) antibody. CD96 expression on CD4+ and CD8+ cells, NK cells and few B cells is demonstrated in FIG. 1A & 1B. NK, CD4+ and CD8+ T cells also express CD226 (FIG. 1C). Around 40% of NK cells and T cells co-express CD226 and CD96 (FIG. 1D). A higher percentage of T cells expressing CD226 co-express CD96 in comparison to NK cells (FIG. 1E).

More specifically, we have found that among peripheral blood mononuclear cells (PBMCs) of healthy donors, 38.5% (median, range from 55-22.5%, n=8) of NK cells, 39.1% (53.2-29%, n=4) of CD4⁺ T cells and 45.6% (53.8-26.2%, n=4) of CD8⁺ T cells co-express CD96 and CD226. Interestingly, 51% (75-25%, n=8) of NK cells, 96.8% (99.6-96.5%, n=4) of CD4⁺ T cells and 73.1% (87.7-68.1%, n=4) of CD8⁺ T cells expressing CD226 were found to co-express CD96. Of note, 27% (45-20%, n=4) of NK cells, 15.5% (20-8%, n=4) of CD4⁺ T cells, 28% (30-16%, n=4) of CD8⁺ T cells expressing CD226 and CD96 also co-express TIGIT (data not shown).

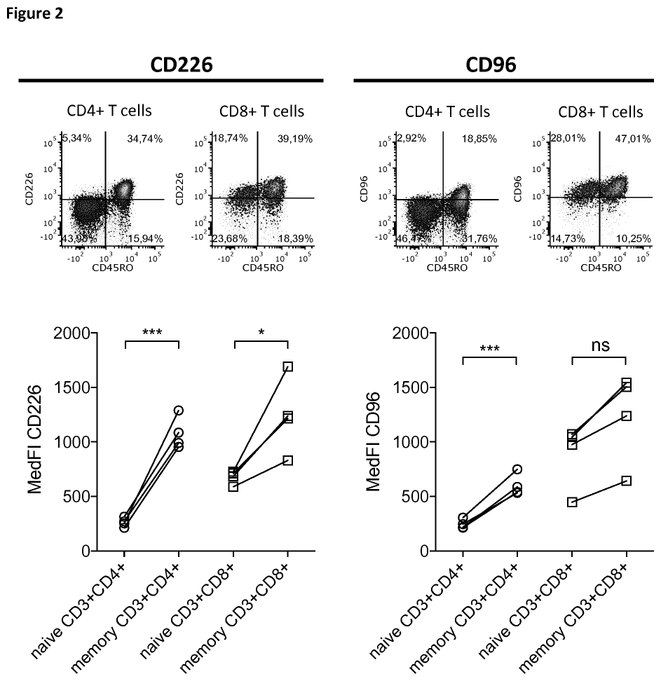

FCM analysis further showed that, like CD226, CD96 was more strongly expressed on CD45RO+ memory T cells than on resting naive T cells (FIG. 2).

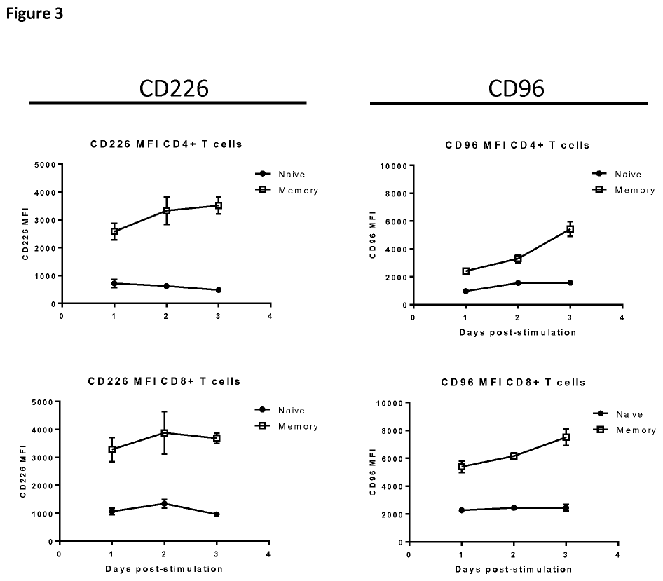

As observed for CD226 (Lozano et al. The TIGIT/CD226 Axis Regulates Human T Cell Function. J Immunol. 188:3869-3875 (2012)), the expression of CD96 was increased on both CD4+ and CD8+ memory T cells activated with anti-CD3 antibody (OKT3), but not on naive T cells in this activation condition (FIG. 3).

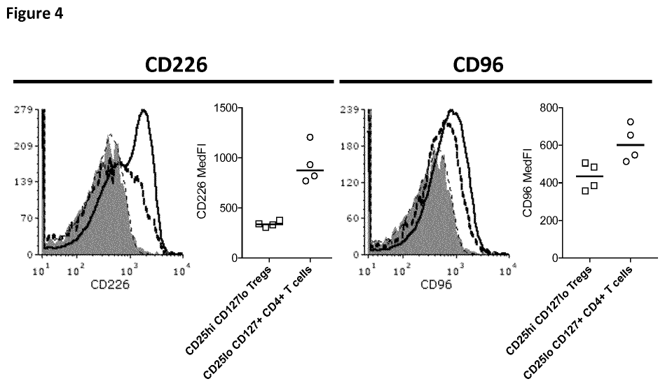

Finally, the expression of CD96 was investigated by FCM on circulating CD4+ regulatory T cells (Tregs) identified by the high expression of CD25 and low expression of CD127 markers. As shown in FIG. 4, CD96 was found to be less expressed on CD4+ Treg cells in comparison to conventional CD4+ T cells (CD25lo/CD127+ cells), similarly to CD226 (Fuhrman et al. Divergent Phenotypes of Human Regulatory T Cells Expressing the Receptors TIGIT and CD226. J Immunol. 195(1):145-155 (2015)). Therefore, it is expected that the use of an agonistic anti-CD96 therapeutic antibody will preferentially co-stimulate conventional T cells rather than regulatory T cells.

1.2 CD96 can Form Homodimers and Heterodimers with CD226

It has been described that CD226 can form cis-homodimers that are required for the efficient interaction of CD226 with CD155 and for the activation of cells expressing CD226. CD226 can also form cis-heterodimers with TIGIT that results in the disruption of CD226 homodimerization and thus CD226 activation, most likely through inhibition of CD226/CD155 interaction (Johnston et al. The Immunoreceptor TIGIT Regulates Antitumor and Antiviral CD8+ T Cell Effector Function. Cancer cell 26(6):923-937 (2014)). However, whether CD96 can form homodimers and/or heterodimers with CD226 is not known. We therefore investigated the homo and hetero-dimerization of hCD96 with hCD96 and hCD226 by using fluorescence resonance energy transfer (FRET) between antibodies tagged with the phycoerythrin (PE—excited by 488 nm laser) and the allophycocyanin (APC—detected using the emission filter 670 LP) (Batard et al. Use of phycoerythrin and allophycocyanin for fluorescence resonance energy transfer analyzed by flow cytometry: advantages and limitations. Cytometry 48(2):97-105 (2002)). We expressed transiently on CHO cells the hCD226-His tagged protein in combination or not with the hCD96-HA and/or with hTIGIT-Myc tagged proteins by transfection with the plasmids HG10565-NH, HG11202-NY, HG10917-NM respectively (SinoBiological). The CD226, CD96 and TIGIT were labelled with anti-hCD226, -hCD96, -hTIGIT, -His, -HA or -Myc mAbs coupled to PE and APC. As control, cells are separately labelled with either anti-Tag mAbs-PE or anti-Tag mAbs-APC, then pooled. Transfected cells were incubated at +4° C. for 30 minutes with mAbs, then after several washes, the interaction between molecules (FRET between PE-conjugated mAbs and APC-conjugated mAbs) was analyzed by flow cytometry on a FACSCanto II flow cytometer (BD biosciences). Of note, for each condition of transfected, four combinations of mAbs have been tested, ie. Anti-Tag-PE+ anti-Tag-APC; Anti-Tag-PE+ anti-receptor-APC, anti-receptor-PE+ anti-receptor-APC, and anti-receptor-PE+ anti-Tag-APC. As described by Batard et al, only some combinations of mAbs are able to allow energy transfer (data not shown) (Batard, 2002).

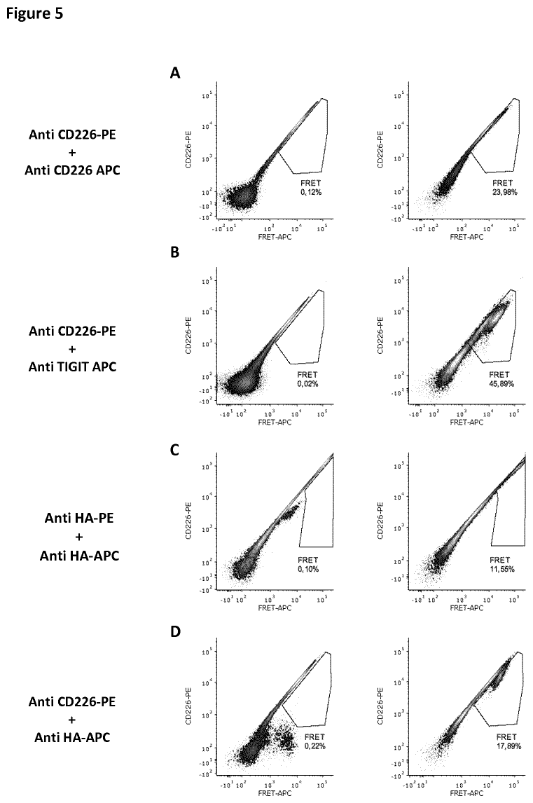

We confirmed the hCD226 formed hCD226-hCD226 homodimers (FIG. 5A) and heterodimers with hTIGIT (FIG. 5B). Of importance, we observed that hCD96 was able to form hCD96-hCD96 homodimers (FIG. 5C) as well as heterodimers with hCD226 (FIG. 5D). Thus, like TIGIT, CD96 can form a heterodimer with CD226 which may either lead to a new mechanism of inhibition of the CD226-CD155 interaction or represent a new mechanism of activation (co-stimulation) of the cells expressing CD226 and CD96 through interaction with CD155.

As already described in the literature, the expression of CD96 has been confirmed on circulating NK, T and NKT cells of healthy individuals. Moreover, CD96 was found to be co-expressed with CD226 on the majority of the NK, T and NKT cells. On circulating NK cells, we have now observed that CD96 is expressed on resting and activated CD56dim and CD56bright/CD1610 subsets, while TIGIT is not expressed on the CD56bright/CD1610 subset (data not shown). On circulating T cells, we have shown that CD96 is mostly expressed on CD4+ memory T cells and on both naive and memory CD8+ T cells, like CD226. The expression of CD96 is further increased on anti-CD3-activated memory T cells, like CD226. Finally, we have observed that the expression of CD96 is weaker on the circulating CD4+ Treg population compared to conventional CD4+ T cells.

We have also demonstrated that human CD96 can form homodimers as well as heterodimers with human CD226. This dimerization may play a key role in the functional activities of CD96 on cells expressing CD96 and CD226.

Altogether, these observations combined with our results showing that some of the disclosed anti-CD96 antibodies co-stimulate human T cells (see below), suggest that an agonistic anti-CD96 antibody preferentially stimulates conventional CD8+ and CD4+ T cells and thus finds use for increasing cellular immune responses in patients with diseases such as cancer. The stimulating activity of CD96 may be mediated by triggering of CD96 as a monomer or homodimer, and/or as heterodimer with the activating receptor CD226.

2. Generation of Mouse Anti-Human CD96 Antibodies

2.1 The human CD96 used for the assay, unless otherwise mentioned, is the shorter form of CD96 (isoform v2 lacking the exon 4), which is the major form expressed on human lymphocytes.

In order to generate mouse antibodies against human CD96, mice were immunized by applying a protocol comprising 4 DNA injections at 2 weeks' intervals followed by two final boosts at one-week interval and animal sacrifice according to sera screening results:
5 WT mice: Prime humanCD96 DNA+Boost humanCD96 DNA
5 WT mice: Prime humanCD96 DNA+Boost humanCD96 CHO (CHO cells transfected with hCD96 expression vector)

The presence and the titer of IgG binding to human CD96 was monitored in serum of immunized animals by ELISA (coating of hCD96-humanIgG1 fusion protein, hCD96-hFc, produced and purified in house) and by flow cytometry on CHO cells expressing hCD96. The presence of mAbs inhibiting the CD155-CD96 interaction was also tested using interference ELISA. Animals displaying strong anti-CD96 IgG titers were sacrificed. Their spleens and lymph nodes were extracted and the mononuclear cells (MNCs) were purified and frozen. The cells from the 5 mice boosted with humanCD96-CHO were pooled.

2.2 Single B Cell Screening Using the ISAAC Technology and Generation of Recombinant Anti-CD96 Antibodies

The ISAAC technology, described in WO2009/017226, is a unique method for detecting individual antibody secreting cells using microarray chips, which enables the analysis of live cells on a single-cell basis and offers a rapid, efficient and high-throughput (up to 234,000 individual cells) system for identifying and recovering the relevant cells.

An array of single live cells was prepared by applying mouse MNCs, previously purified from spleen and lymph nodes of immunized mice, to a microarray chip. The chip surface was previously coated with the target antigen (hCD96-hFc) and the anti-CD96 mouse antibodies secreted by an antibody secreting cell were trapped by the CD96 coated on the surface around the well. After washings, the presence of mouse IgG bound to the immobilized CD96 was detected by an anti-mouse IgG antibody coupled to Cy3 and fluorescence microscopy. Binding of the antigen to the specific antibodies formed distinct circular spots, which were easily distinguishable from nonspecific signals. CD96-specific antibody secreting single cells were then retrieved by micromanipulation, mRNA was recovered from single cells and cDNA sequences coding the variable regions of the heavy (VH) and light (VL) chain of IgG were amplified by RT-PCR. The VH and VL sequences were then cloned in expression vectors containing a mouse gamma-2a constant region (Fcγ2a) and a kappa constant region, respectively. Antibodies were also cloned in a mIgG2a format containing the D265A mutation (mIgG2a-D265A) that decreases the affinity of mIgG2a for Fcγ receptors.

After co-transfection of the H and L chain expression vectors in CHO cells, the recombinant antibodies were tested for confirmation of CD96 recognition and specificity by ELISA on plates coated with hCD96-hFc or with an unrelated human protein fused to hFc and produced and purified as for the hCD96-hFc protein. The inhibition of CD155-CD96 interaction was monitored by ELISA. Recognition of native CD96 and inhibition of CD96-CD155 interaction was confirmed by flow cytometry on CHO cells transfected with hCD96 or an irrelevant protein.

In total, 160 pairs of VH and VL were amplified and the resulting IgG antibodies were confirmed for specific binding to human CD96. Among them, 19 antibodies were produced and purified. From those 19 antibodies, 12 inhibited hCD155-hCD96 interaction.

In Vitro Characterization of Recombinant Mouse Anti-CD96 Antibodies

FACS Binding on CHO Cells Expressing Human CD96

The capacity of the identified mouse anti-CD96 antibodies to recognize the cell membrane expressed human CD96 was further analyzed by flow cytometry (FCM) by using CHO cells stably transfected with the CD96 short isoform v2 (CD96v2; Genbank accession number NM_005816, flanked in C-terminus with HA-tag) (SEQ ID NO: 268). The recognition of the long form of human CD96 (v1 isoform; CD96v1) (SEQ ID NO: 267) was also tested by flow cytometry with CHO cells transiently expressing the human CD96v1 (Genbank accession number NM_198196.2). The binding capacity of the 19 candidates was compared to the binding capacity of the 2 mIgG1 benchmark antibodies NK92.39 (Fuchs, 2004; Biolegend) and clone 628211 (R&D System).

Mouse anti-CD96 antibodies and isotype control antibodies were incubated at various concentrations from 10 μg/mL to 4.6 ng/mL with CHO cells expressing human CD96 at +4° C. for 30 minutes. After two washings, the presence of antibody bound to cell membrane CD96 was revealed by incubation with a PE-coupled anti-mouse antibody and analysis on an Accuri-C6 flow cytometer (BD-Biosciences). The differences between the mean fluorescence intensity (MFI) obtained for each of the antibody concentration and the intensity obtained in absence of primary antibody (delta-MFI), were calculated and plotted against the concentrations of antibodies. Negative control antibodies of appropriate isotype that did not recognize CD96 were tested in the same conditions to measure the background of antibody staining (non-specific staining).

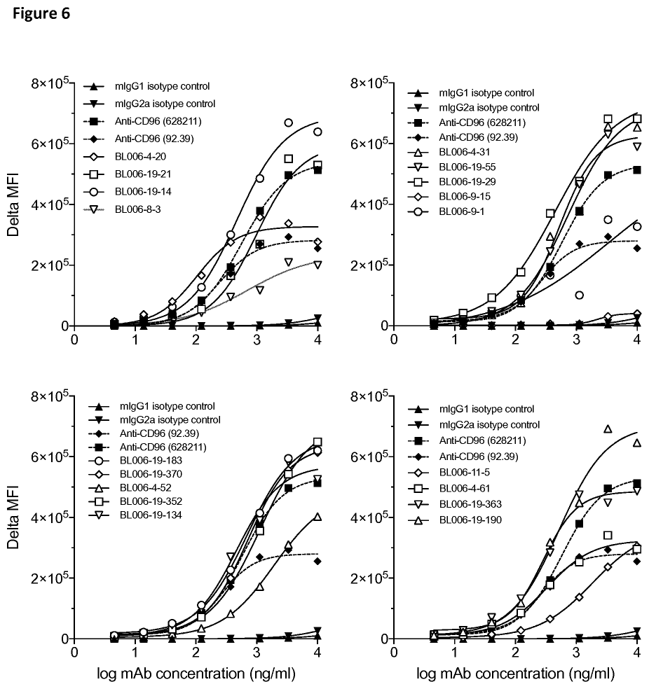

The binding results obtained for 19 mouse anti-hCD96 antibodies are shown in FIG. 6 and summarized in Table 6 and are compared with the results obtained for the benchmark antibodies.

Among the 19 antibodies tested, 11 candidates were found to bind strongly to hCD96v2 (BL006-4-31, BL006-19-183, BL006-19-190, BL006-19-134, BL006-19-21, BL006-19-55, BL006-19-352, BL006-19-363, BL006-19-370, BL006-19-14, BL006-19-29). Binding of these candidates to hCD96v2 was similar or superior to the one of clone 628211. Six candidates (BL006-4-20, BL006-4-52, BL006-4-61, BL006-11-5, BL006-8-3, BL006-9-1) bound weaker to CD96v2, as did benchmark NK92.39. Candidate BL006-9-15 bound even weaker to CD96v2. Finally, candidate BL006-2-19 showed very weak binding to CD96v2 by FCM (not shown).

Among the 19 antibodies tested, 11 candidates were also found to strongly bind to the long form of human CD96 (CD96v1) expressed on CHO cells by FCM (BL006-4-31; BL006-19-21; BL006-19-183, BL006-19-14, BL006-19-29, BL006-4-52; BL006-2-19, BL006-11-5, BL006-4-61, BL006-9-1, BL006-8-3, BL006-9-15). The rest of the candidates bound weaker to the long form of hCD96 (CD96v1), as did the 2 benchmarks NK92.39 and 628211.

TABLE 6
EC50 values and maximum binding capacity of anti-CD96
antibodies on hCD96-transfected CHO cells by flow cytometry
analysis (values of 1 representative experiment)

| | EC50 on hCD96 | Maximum binding | Recognition |
|---|---|---|---|
| | short form | on hCD96 short | hCD96 long |
| Antibodies | (nM) | (delta MFI) | form |
| 628211 | 3.75 | 543 390 | +/− |
| NK92.39 | 1.59 | 282 606 | +/− |
| BL006-4-20 | 0.69 | 327 585 | +/− |
| BL006-4-31 | 4.82 | 723 334 | + |
| BL006-19-21 | 6.53 | 598 872 | + |
| BL006-19-183 | 4.56 | 681 369 | + |
| BL006-19-190 | 3.39 | 710 819 | +/− |
| BL006-19-134 | 3.00 | 575 690 | +/− |
| BL006-19-55 | 3.30 | 635 919 | +/− |
| BL006-19-352 | 6.87 | 731 367 | +/− |
| BL006-19-363 | 1.69 | 494 147 | +/− |
| BL006-19-370 | 4.71 | 648 271 | +/− |
| BL006-19-14 | 3.10 | 693 852 | + |
| BL006-19-29 | 2.98 | 753 029 | + |
| BL006-4-52 | 13.41 | 492 761 | + |
| BL006-2-19 | Not determined* | Not determined* | ++ |
| BL006-11-5 | 12.83 | 373 445 | ++ |
| BL006-4-61 | 2.05 | 329 658 | ++ |
| BL006-9-1 | 17.46 | 513 596 | ++ |
| BL006-8-3 | 4.68 | 234 883 | ++ |
| BL006-9-15 | 14.26 | 42 028 | ++ |

*antibody BL006-2-19 bound very weakly on hCD96v2 by FCM. The EC50 and maximum binding values could not be calculated;
+/−: weak binding at 10 μg/ml antibody; +: binding at 10 μg/ml antibody; ++: strong binding at 10 μg/ml antibody.

3.2 Cross-Reactivity with Mouse and Rhesus CD96

The capacity of the identified mouse anti-human CD96 antibodies to recognize the CD96 protein from other species was further analyzed by flow cytometry by using CHO cells expressing the mouse or the rhesus CD96. The expression of the species-specific CD96 on CHO cell surface was confirmed by using staining with appropriate anti-CD96 antibodies and flow cytometry on non-fixed cells. The rat anti-mCD96 antibody clone #630612 (R&D Systems) and the candidate BL006-9-1 (positive by ELISA on rhesus CD96) were used to confirm the expression of rhesus CD96, while the clone 6A6 (rat IgG2a, e-Bioscience) was used to verify the expression of mouse CD96.

Mouse anti-CD96 antibodies and isotype control antibodies were incubated at 10 or 1 μg/mL with CHO cells expressing different CD96 species at +4° C. for 30 minutes. After 2 washings, the presence of antibody bound to cell membrane CD96 was revealed by incubation with a PE-coupled anti-mouse antibody and analysis on an Accuri-C6 flow cytometer (BD-Biosciences).

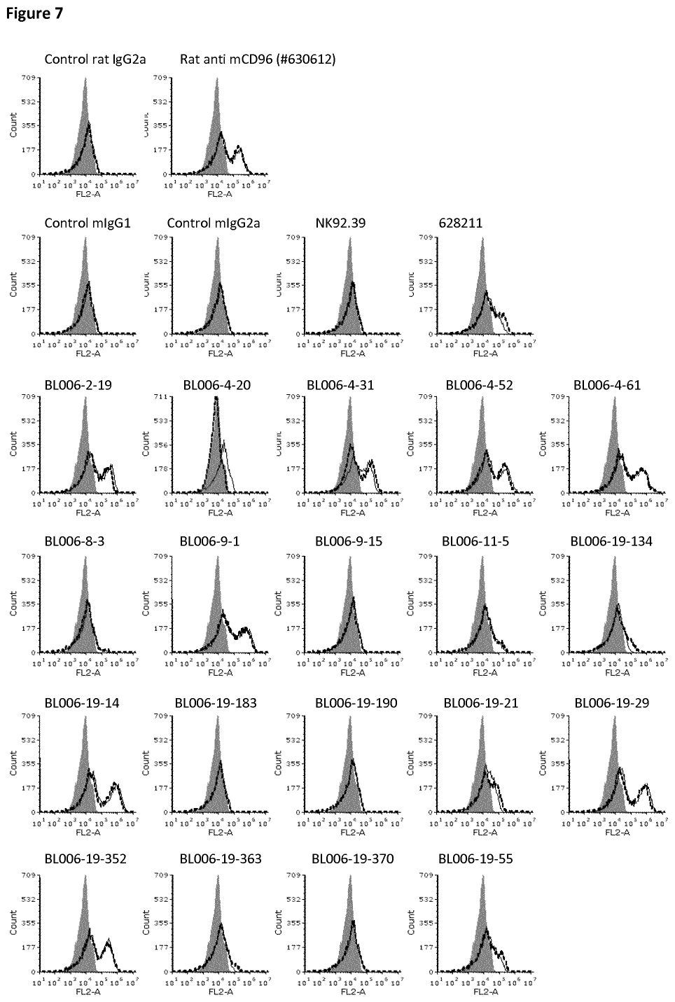

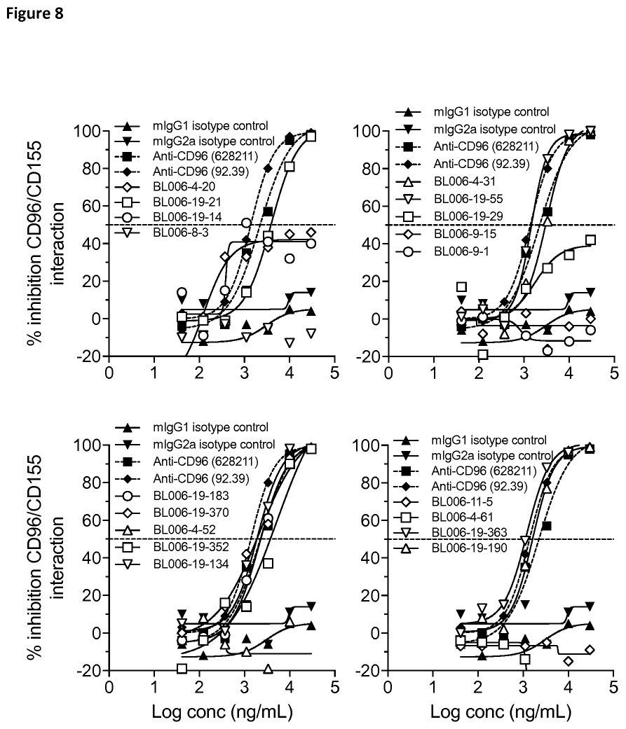

The results obtained for the 19 mouse anti-CD96 antibodies are shown in the FIG. 7 and compared with the results obtained with the benchmark antibodies. Among the 19 candidates tested, 8 antibodies strongly recognized rhesus CD96 (BL006-4-31, BL006-19-352, BL006-19-14, BL006-19-29, BL006-4-52, BL006-2-19, BL006-4-61, BL006-9-1). The 2 candidates BL006-19-21 and BL006-19-55 bound weaker to rhesus CD96, as did antibody 628211. The 9 remaining candidates and the benchmark NK92.39 did not bind to rhesus CD96. Of note, none of the 19 candidates recognized mouse CD96 by FCM (data not shown).

3.3 Inhibition of CD96/CD155 Interaction Measured by Flow Cytometry on CHO Cells

All candidates except BL006-2-19 that bound very weakly on hCD96v2 by FCM were tested for their capacity to inhibit the binding of human CD155 to hCD96v2 stably expressed on CHO cells. Human CD96-transfected CHO cells (3×10⁵ cells/well of 96-well plate) were incubated at +4° C. for 30 minutes with serial dilutions of anti-CD96 antibodies or isotype control antibody (in mIgG2a or mIgG2aD265A format) in the presence of 4 μg/mL of biotinylated hCD155-Fc (produced and biotinylated at BliNK). The cells were then washed twice and the binding of CD155-hFc to CHO cells was revealed by incubation with a FITC-conjugated Streptavidin (BD Biosciences) and flow cytometry analysis on an Accuri-C6 flow cytometer (BD biosciences).

The percentage of inhibition of hCD155 binding to hCD96 was calculated as follows:

% inhibition=(1−(MFI_wAb&CD155−MFI_CHO)/(MFI_wCD155−MFI_CHO))×100, where MFI_wAb&CD155 is the Mean Intensity Fluorescence (MFI) obtained with the hCD96-CHO cells incubated with the tested antibody and hCD155-hFc; MFI_CHO is the MFI obtained in the absence of hCD155 and mAbs; and MFI_wCD155 is the fluorescence with hCD155 but without pre-incubation with antibody (0% CD155 binding inhibition). The % of inhibition was plotted against antibody concentration and the IC50 values were calculated by using a nonlinear regression analysis model of the GraphPad Prism software.

The results illustrated in FIG. 8 and Table 7 show that 9 candidates strongly inhibited the binding of hCD155 to hCD96v2 expressed on CHO cells, with IC50 values ranging from 5.9 to 19.4 nM (candidates BL006-4-31, BL006-19-21, BL006-19-183, BL006-19-190, BL006-19-134, BL006-19-55, BL006-19-352, BL006-19-363, BL006-19-370). Those 9 anti-CD96 candidates were similar to the clone 628211 and NK92.39 antibodies. Candidates BL006-4-20, BL006-19-14 and BL006-19-29 also inhibited CD155 binding to CD96v2 but with a lower potency since they did not reach 50% of binding inhibition at 30 μg/mL (200 nM). Finally, 6 candidates BL006-4-52, BL006-4-61, BL006-11-5, BL006-8-3, BL006-9-1 and BL006-9-15 did not inhibit CD96v2/CD155 interaction at 30 μg/mL (200 nM).

TABLE 7
IC50 values of anti-CD96 antibodies in the CD96/hCD155 inhibition
assay with CD96v2-transfected CHO cells and flow cytometry
analysis (mean of n = 2 independent experiments)

| | | Maximal Inhibition Capacity |
|---|---|---|
| Antibody | IC50 (nM) | (%) |
| 628211 | 15.78 | 100 |
| NK92.39 | 10.45 | 100 |
| BL006-4-20 | 2.81* | 43.8 |
| BL006-4-31 | 16.78 | 100 |
| BL006-19-183 | 15.04 | 100 |
| BL006-19-190 | 11.53 | 98.1 |
| BL006-19-134 | 11.37 | 100 |
| BL006-19-21 | 19.42 | 100 |
| BL006-19-55 | 8.84 | 98.3 |
| BL006-19-352 | 14.68 | 100 |
| BL006-19-363 | 5.92 | 100 |
| BL006-19-370 | 10.85 | 100 |
| BL006-19-14 | 2.67* | 41.4 |
| BL006-19-29 | 5.24* | 43.1 |
| BL006-4-52 | nd | <5 |
| BL006-4-61 | nd | <5 |
| BL006-11-5 | nd | <5 |
| BL006-8-3 | nd | <5 |
| BL006-9-1 | nd | <5 |
| BL006-9-15 | nd | <5 |

*relative IC50;
nd: not determined

Binding of Anti-CD96 Candidates on Human PBMCs

The anti-CD96 mAbs candidates have been tested for their binding capacity on human PBMCs. The mAbs were previously biotinylated (EZ-link Sulfo-NHS-LC-Biotin). PBMCs from 3 healthy donors were stained with anti-CD3-PerCP (Clone SK7, BD biosciences), CD56-FITC (Clone REA196 Miltenyi), and anti-CD96-Biotinylated (10 μg/ml). The T cells population was gated as CD3pos, CD56neg (mean: 64.9%, SD: 9.4% of lymphocyte population), NK cell as CD3neg, CD56pos (mean: 11.1%, SD: 1.7% of lymphocyte population), and NKT cells, as CD3pos, CD56pos (mean: 4.2%, SD: 2.9% of lymphocyte population). Streptavidin-APC (Abcam) was used to detect anti-CD96-biotinylated mAbs.

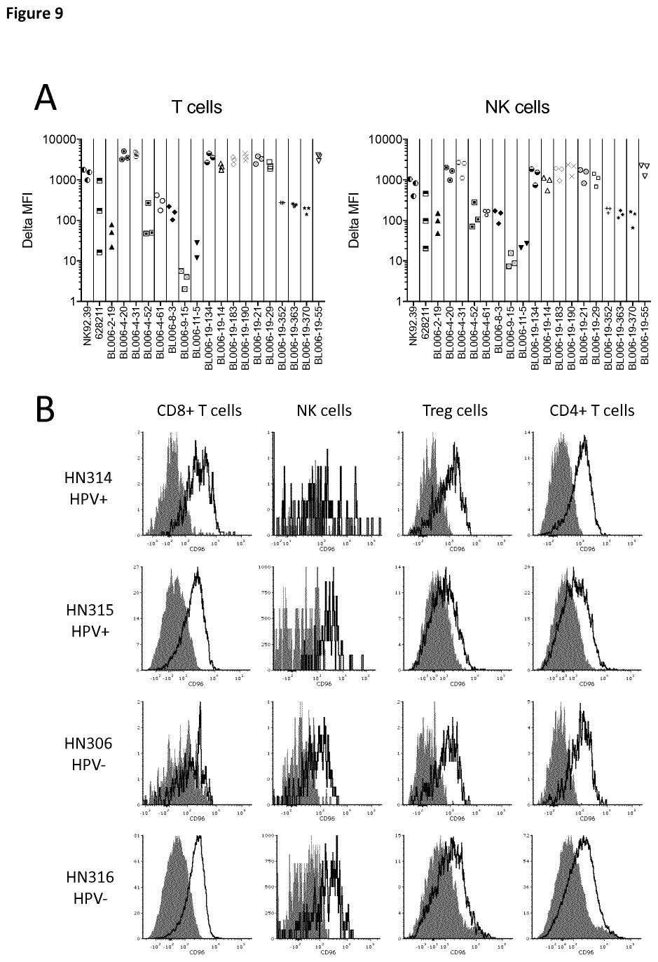

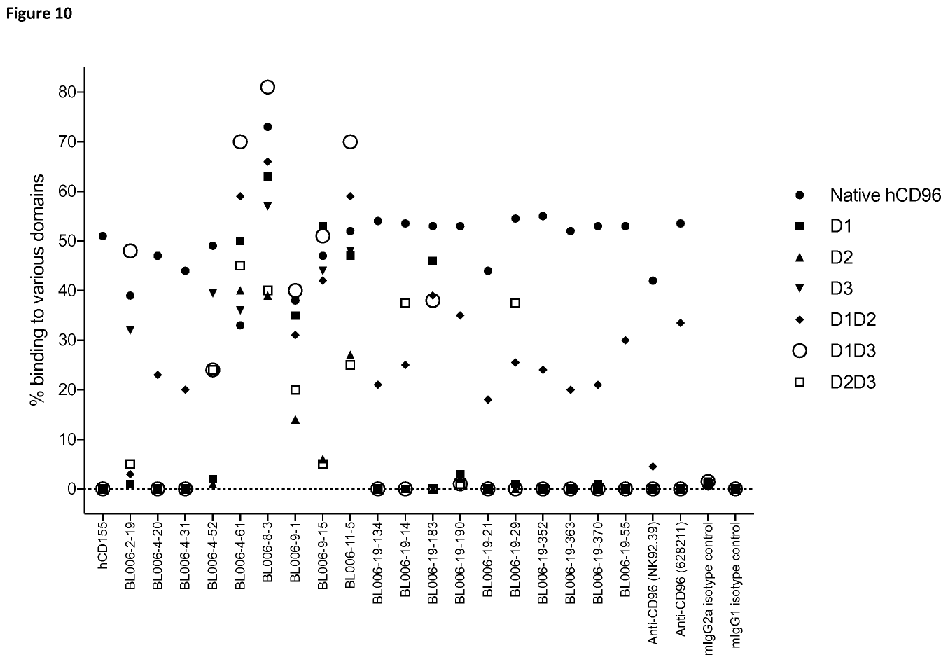

Among the 18 antibodies tested (BL006-9-1 was not tested)), 9 candidates (BL006-4-20, BL006-4-31, BL006-19-29, BL006-19-14, BL006-19-55, BL006-19-183, BL006-19-190, BL006-19-134, BL006-19-21) displayed strong binding on T and NK cells (FIG. 9A), as well as on NKT cells (not shown). Seven candidates (BL006-2-19, BL006-8-3, BL006-4-61, BL006-19-352, BL006-19-363, BL006-19-370, BL006-4-52) displayed a weaker binding on NK, T and NKT cells (FIG. 9A and not shown), and 2 showed very weak binding on NK, T and NKT cells (BL006-11-5 and BL006-9-15).

The expression of CD96 has been tested on human tumor infiltrating lymphocytes (TILs) obtained from head and neck tumors. To extract TILs, head and neck tumors were washed twice with serum free RPMI media and any blood clots removed with tweezers. Tumor samples were first mechanically disaggregated using sterile scalpel blade to dice into small pieces, then enzymatically digested using DNAse I (800 U/mL) and Liberase Dispase Low (0.15 WU/mL) for 20 minutes at 37° C. with continuous agitation. Enzyme reaction was quenched with 5 ml RMPI 10% FCS and disaggregated tumor samples were passed through a 100 μm cell strainer. Prior to counting, red blood cells were removed using red cell lysis buffer at room temperature for 5 minutes. TILs, obtained from 4 patients, were stained anti-CD3-eFluor450 (UCHT1, ebioscience), anti-CD56-PC7 (CMSSB, ebioscience), anti-CD4-APC-eFluor780 (RPA-T4, ebioscience), anti-CD8-FITC (RPA-T8, Biolegend), anti-FoxP3-PE (PCH101(CL), ebioscience) and anti-CD96-APC (FAB6199A, R&D Systems). CD96 was markedly expressed on CD3+CD8+ T cells (except for donor HN306), CD3+CD4+ conventional T cells, CD3-CD56+NK cells and CD3+CD4+FoxP3+ cells from 4 patients (FIG. 9B). Of note, the presence of CD96 on the various lymphocyte subsets tested, was not related to the HPV status of these tumors.

CD96 is not Expressed on Human Platelets and Our Anti-CD96 Candidates do not Bind on Platelets

CD226 has been described to be expressed on platelets and this may represent a potential side effect for the use of therapeutic anti-CD226 antibodies. In contrast, CD96 is not expressed on human platelets (Wang, 1992). The potential binding of our 19 anti-CD96 candidates on platelets was further tested by FCM. None of the candidates as well as benchmarks NK92.39 and 628211 show significant binding to human platelets (data not shown). Thus, in contrast to CD226, CD96 is not expressed on human platelets and our anti-CD96 candidates do not show reactivity on platelets. In conclusion, conversely to CD226, agonistic antibodies against CD96 can be safely developed without incurring the risk to trigger platelets aggregation.

Epitope Mapping by FCM on hCD96 Truncated Proteins

Expression vectors coding for truncated extracellular domains of hCD96v2 (short isoform v2) were constructed to map, within the 3 described immunoglobulin domains of hCD96v2, the epitopes recognized by the anti-hCD96 candidates. All expression vectors constructed contained the signal peptide, the D4 domain, the transmembrane, the intracellular chain of hCD6 plus a HA-Tag to monitor transfection efficiency of the various constructs. The expression vectors contained only D1, D2, D3 or combinations of those 3 domains (D1D2, D1D3, D2D3). The anti-hCD96 antibodies were tested by FCM for their capacity to recognize the truncated CD96 proteins transiently expressed on CHO-S cells. As shown in FIG. 10, all the anti-CD96 antibodies tested recognized the native hCD96v2 protein transiently expressed in CHO-S cells. Of the 19 antibodies tested, 9 candidates and the 2 reference anti-CD96 antibodies (NK92.39 and 628211) displayed specific binding to CHO-S cells expressing the construct of the fusion of D1 and D2 domains (BL006-4-20, BL006-4-31, BL006-19-134, BL006-19-190, BL006-19-21, BL006-19-352, BL006-19-363, BL006-19-370 and BL006-19-55), 2 candidates displayed specific binding to CHO-S cells expressing the construct of the fusion of D1 and D2 domains and the fusion of D2 and D3 domains (BL006-19-14 and BL006-19-29), 1 candidate displayed specific binding to CHO-S cells expressing the construct of D1 domain, the fusion of D1 and D2 domains and the fusion of D1 and D3 domains (BL006-19-183), 2 candidates displayed specific binding to CHO-S cells expressing the construct of D3 domain, the fusion of D1 and D3 domains and the fusion of D2 and D3 domains (BL006-4-52 and BL006-2-19), and 5 candidates displayed specific binding to CHO-S cells expressing all the constructs tested (BL006-4-61, BL006-8-3, BL006-9-1, BL006-9-15 and BL006-11-5). These results indicate that (i) both references anti-CD96 antibodies and candidates BL006-4-20, BL006-4-31, BL006-19-134, BL006-19-190, BL006-19-21, BL006-19-352, BL006-19-363, BL006-19-370 and BL006-19-55 recognize epitopes comprised within D1 and D2 domains; (ii) candidates BL006-19-14 and BL006-19-29 recognize epitopes comprised within the D2 domain; (iii) candidate BL006-19-183 recognizes an epitope comprised within the D1 domain; (iv) candidates BL006-4-52 and BL006-2-19 recognize epitopes comprised within the D3 domain; (v) while candidates BL006-4-61, BL006-8-3, BL006-9-1, BL006-9-15 and BL006-11-5 are likely to recognize epitopes within the D4 domain.

Affinity of the Best Mouse Anti-CD96 Candidates

The kinetic constants (Kₒₙ and K_{off}) and the dissociation constant (KD) of the anti-hCD96 candidates were measured by surface plasmon resonance (SPR) on Biacore using hCD96-hFc protein (in house production) captured by anti-huFc antibodies coated on the chip and compared to benchmark. Results are shown in Table 8 below. Most of candidates displayed an affinity in the nM range with a KD value between 0.18 and 42.3 nM, except for candidate BL006-2-19 that showed no binding in this setting by Biacore. Ten candidates have better affinity than NK92.39 clone.

TABLE 8
Association (Kₒₙ), dissociation (K_{off}) and affinity (KD) constants
of anti-CD96 antibodies as measured by SPR (Biacore).

| Antibody | KD (M) | Kon (1/Ms) | Koff (1/s) |
|---|---|---|---|
| NK92.39 | 3.24E−09 | 1.07E+05 | 3.46E−04 |
| BL006-4-20 | 8.25E−10 | 6.91E+05 | 5.70E−04 |
| BL006-4-31 | 1.32E−08 | 5.31E+04 | 7.00E−04 |
| BL006-19-183 | 4.50E−10 | 6.56E+05 | 2.96E−04 |
| BL006-19-190 | 2.97E−09 | 1.50E+05 | 4.44E−04 |
| BL006-19-134 | 7.07E−10 | 1.40E+05 | 9.88E−05 |
| BL006-19-21 | 6.75E−09 | 1.46E+05 | 9.88E−04 |
| BL006-19-55 | 3.45E−09 | 1.45E+05 | 5.01E−04 |
| BL006-19-352 | 2.34E−08 | 3.41E+04 | 7.96E−04 |
| BL006-19-363 | 3.50E−09 | 1.67E+05 | 5.85E−04 |
| BL006-19-370 | 4.23E−08 | 4.55E+04 | 1.93E−03 |
| BL006-19-14 | 2.54E−10 | 6.09E+05 | 1.55E−04 |
| BL006-19-29 | 1.82E−10 | 8.96E+05 | 1.63E−04 |
| BL006-4-52 | 2.83E−09 | 1.28E+05 | 3.63E−04 |
| BL006-4-61 | 3.64E−10 | 4.20E+05 | 1.53E−04 |
| BL006-11-5 | 3.47E−09 | 1.67E+05 | 5.79E−04 |
| BL006-8-3 | 3.51E−10 | 5.48E+05 | 1.92E−04 |
| BL006-9-1 | 1.98E−10 | 8.10E+05 | 1.60E−04 |
| BL006-9-15 | 4.60E−09 | 5.98E+05 | 2.75E−03 |

Anti-CD96 Antibody Candidates can Co-Stimulate Human CD4+ and CD8+ T Cells Activation and Proliferation

The biological activity of the anti-CD96 candidates was further evaluated on human T cells co-stimulated with anti-CD3 antibody OKT3. PBMC were isolated from healthy individuals, stained with CFSE fluorescent dye and cultured with soluble or plate-bound anti-CD96 antibodies (mIgG2a-D265A format) in the presence or the absence of different concentrations of soluble anti-CD3 antibody (OKT3, mIgG2a). The cells were collected at different times of culture, and the activation and proliferation of CD4+ and CD8+ T cells was analyzed by FCM by measuring the expression of the CD25 activation marker and the CFSE fluorescence dilution, respectively.

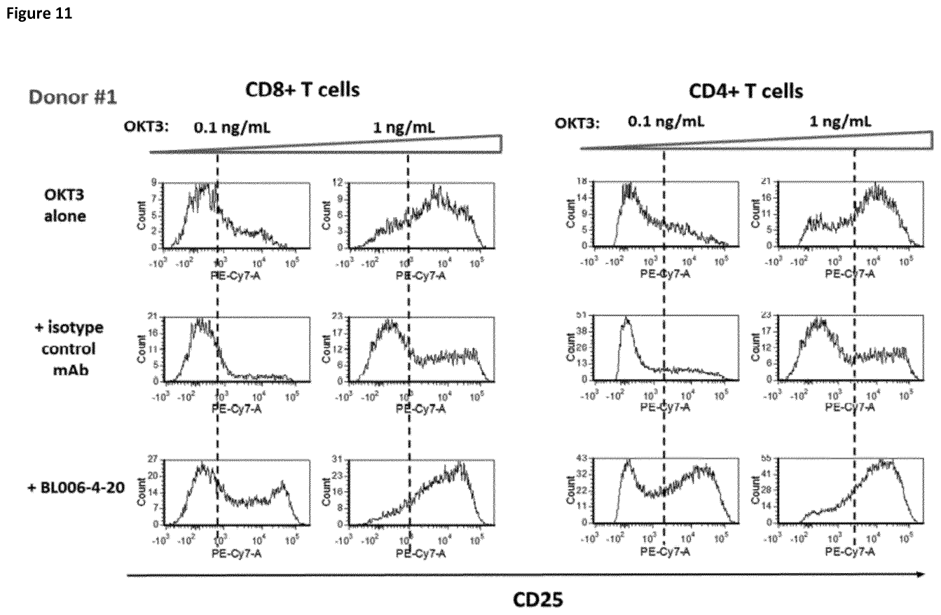

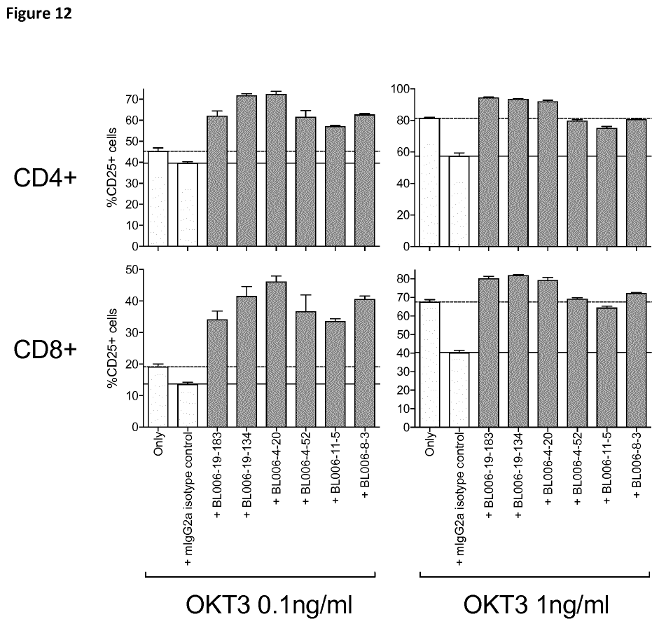

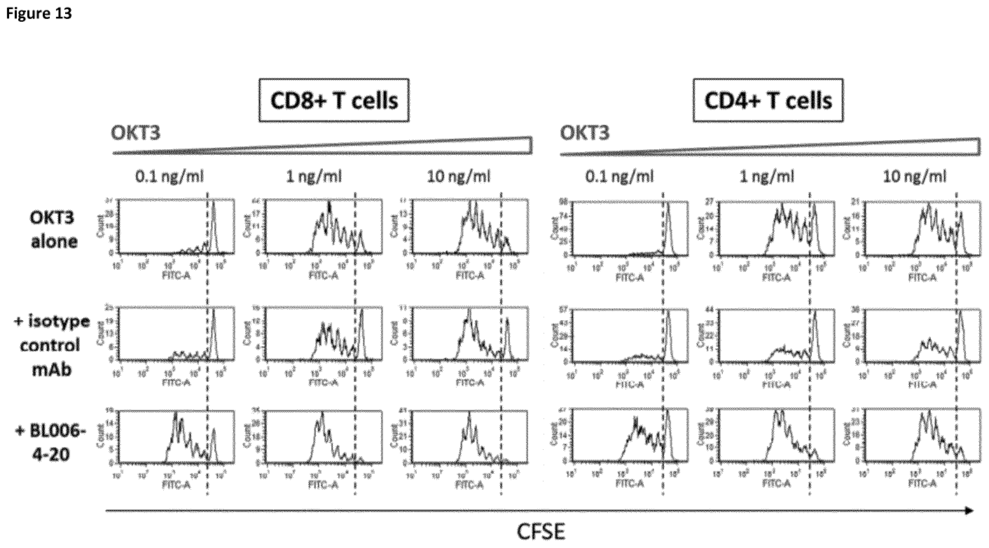

The results reported in FIGS. 11 to 17 below show that, immobilized anti-hCD96 candidates were capable to co-activate both CD4+ and CD8+ T cells with a suboptimal concentration of anti-CD3 antibody. The percentage of activated cells was measured by the increased expression of CD25 (FIGS. 11 and 12) and the dilution of the CFSE staining (FIGS. 13 to 17) that is correlated to the number of dividing cells.

Example of increased CD25 expression on T cells is shown in FIG. 11 for the candidate BL006-4-20 in combination with 0.1 or 1 ng/mL OKT3. The co-stimulation activity of anti-CD96 candidates was already observed for very low concentrations of anti-CD3 antibody (0.1 ng/mL). In comparison with the result obtained with the mIgG2a isotype control, all the 6 candidates tested in the first set of experiments (BL006-4-20, BL006-19-183, BL006-19-134, BL006-4-52, BL006-11-5, BL006-8-3) were found to increase the expression of CD25 on both CD4+ and CD8+ T cells co-stimulated with anti-CD3 (FIG. 12), The BL006-4-20, BL006-19-183 and BL006-19-134 candidates were the most potent at increasing the expression of CD25 on both CD4+ and CD8+ T cells co-stimulated with anti-CD3 (FIG. 12). Similar results were obtained with PBMC purified from 2 additional healthy donors (not shown).

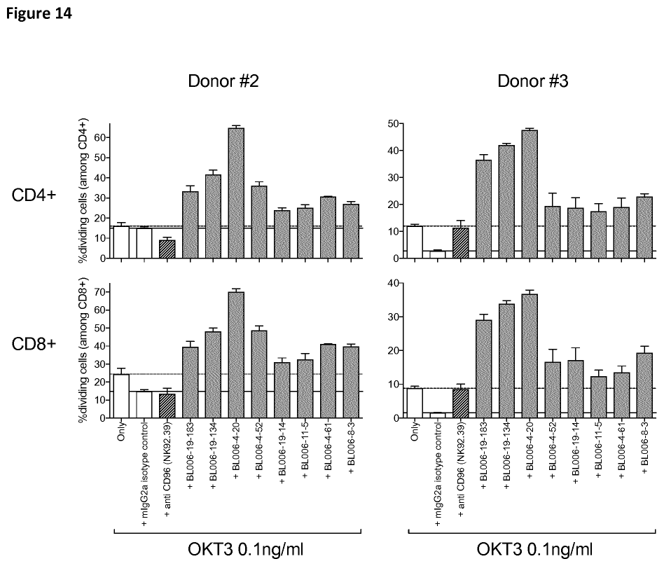

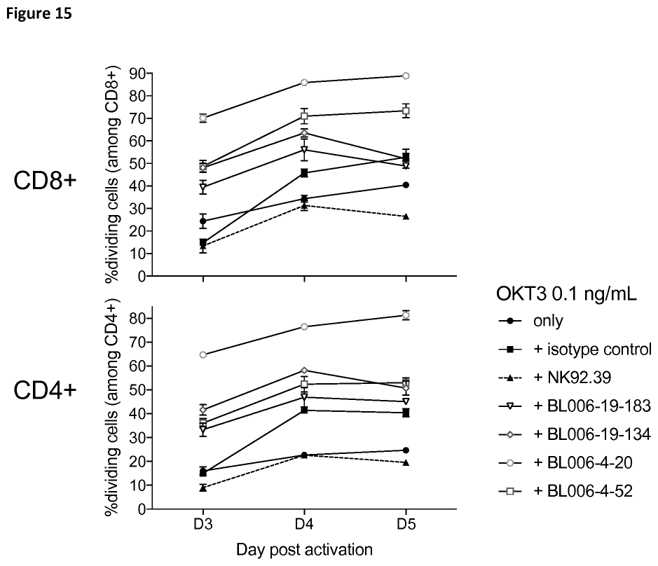

In a second set of experiments, 8 anti-CD96 candidates were tested for their capacity to co-stimulate T cells from 2 donors (#2 and #3) and were compared with the benchmark anti-hCD96 antibody (NK92.39). Example of CFSE dilution measured by FCM is shown in FIG. 13 for the candidate BL006-4-20 in combination with 0.1, 1 or 10 ng/mL OKT3. In comparison to controls (OKT3 only or plus mIgG2a isotype control), all the 8 candidates tested were found to increase the percentage of dividing CD4+ and CD8+ T cells sub-optimally activated with a low concentration of OKT3 (FIG. 14). The candidates BL006-19-183, BL006-19-134 and BL006-4-20 were the most potent at increasing the % of dividing cells in such conditions (FIG. 14). Co-stimulation levels reported as % of CD4+ and CD8+ T cells showing CFSE dilution (as determined by FCM) were less significant for higher OKT3 concentrations (not shown) although the relative proportion of cells having divided 4, 5 or 6 times was higher in presence of the candidate BL-006-4-20 in comparison to controls (FIG. 13). Of importance, the benchmark antibody NK92.39 did not display co-stimulation activity with any of the OKT3 concentrations tested on both donors (FIG. 14). The co-stimulation of CD4+ and CD8+ T cell proliferation by anti-CD96 candidates, but not by NK92.39, was observed for all the time points tested between day 3 to day 5 (FIG. 15).

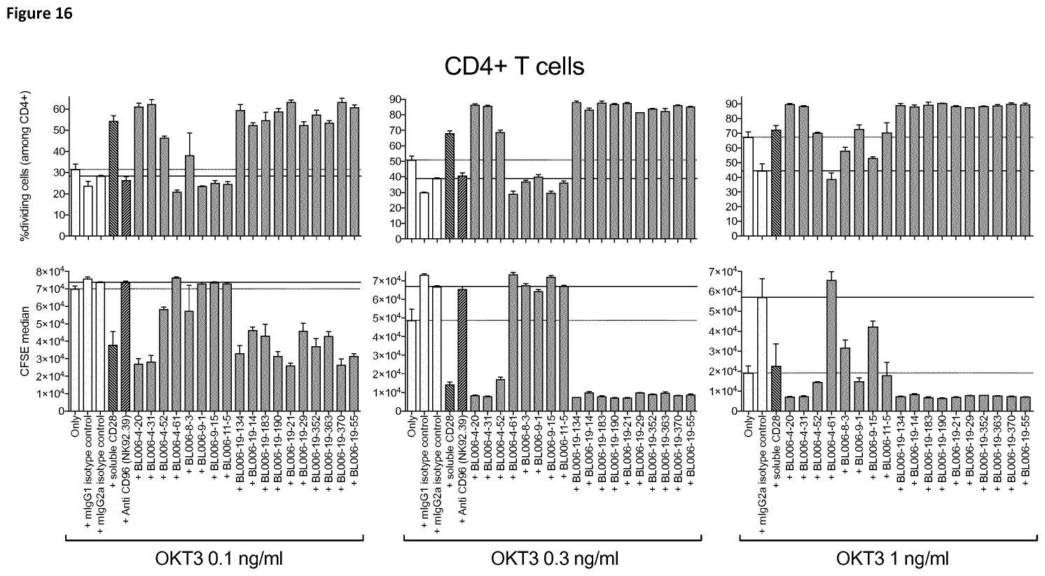

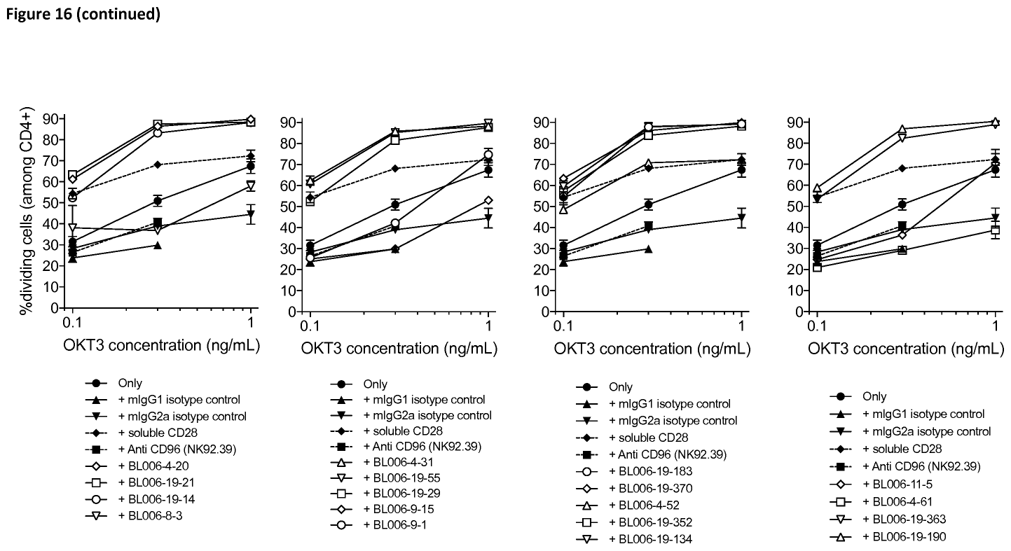

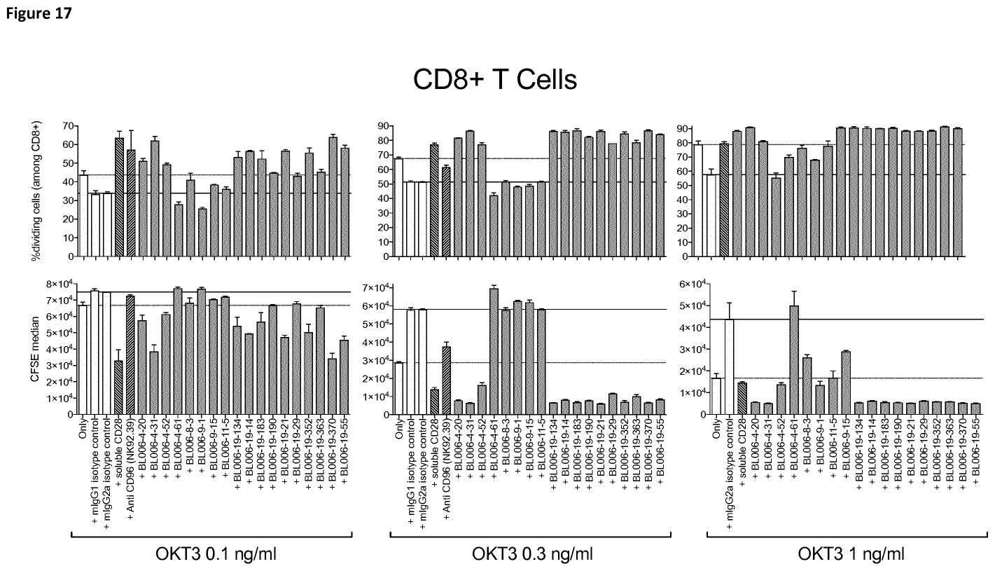

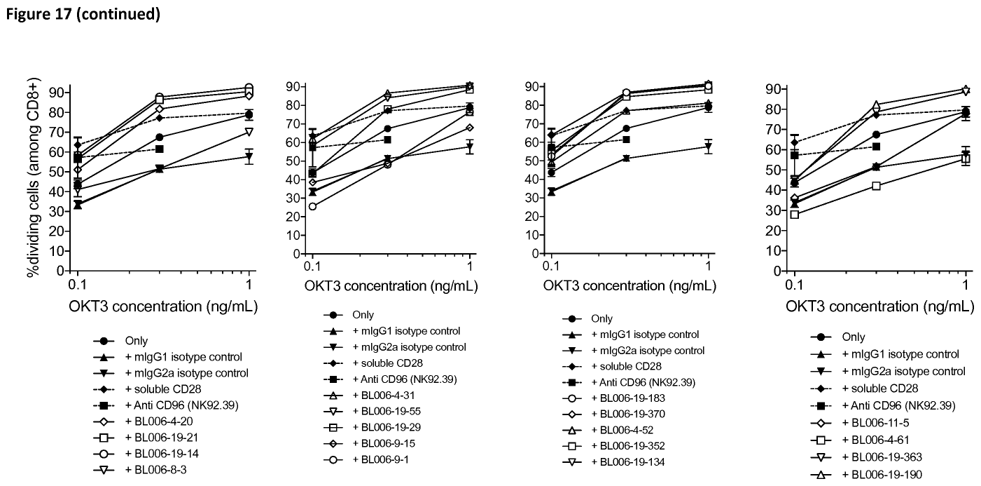

All candidate antibodies, except BL006-2-19, were tested in parallel for co-stimulatory effects on OKT3-sub-optimally activated T cell proliferation. The percentage of dividing cells (cells showing decreased CFSE staining, upper panels) as well as the CFSE median of fluorescence intensities (middle panels) were reported. Lower panels report the percentage of dividing cells for each antibody candidate according to the concentration of OKT3 used for sub-optimal stimulation. Results indicate that candidates BL006-4-20, BL006-4-31, BL006-19-183, BL006-19-134, BL006-19-14, BL006-19-190, BL006-19-21, BL006-19-55, BL006-19-370, BL006-19-363, BL006-19-352, BL006-19-29 and BL006-4-52 co-stimulated CD4+(FIG. 16) and CD8+ T cells (FIG. 17) proliferation, thus confirming previous results for candidates BL006-19-183, BL006-19-134 and BL006-4-20. According to the results observed with controls (OKT3 alone or in presence of isotypes controls), the co-stimulation effect was best observed at 0.1 and 0.3 ng/mL for CD4+ T cells (FIG. 16) and at 0.3 ng/mL for CD8+ T cells (FIG. 17). No significant co-stimulation was observed again with the benchmark anti-hCD96 antibody NK92.39. Of note, the anti-hCD96 candidates were found to co-stimulate T cell proliferation to similar levels to that obtained with an anti-CD28 antibody.

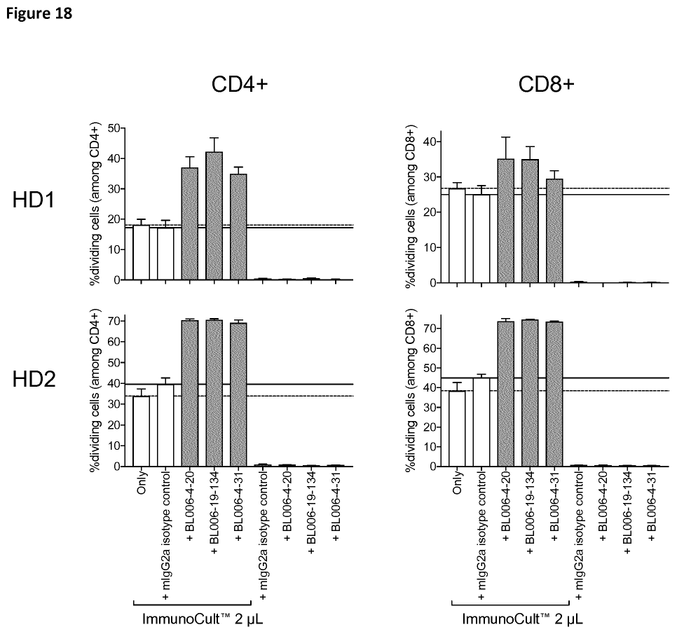

The co-stimulation properties of 3 candidates were tested on purified CD4+ and CD8+ T cells (magnetic cell sorting) in order to evaluate the role of accessory cells present in PBMC on the observed phenomenon. CFSE-stained purified T cells were sub-optimally activated with 2 μL/mL of tetrameric CD3/CD28 complex (ImmunoCult™, Stemcell). The activity of anti-CD96 candidates (10 μg/mL) on these T cells was evaluated 5 days later by FCM. Results indicate that candidates BL006-4-20, BL006-19-134 and BL006-4-31 co-stimulated purified CD4+ and CD8+ T cells (FIG. 18). CD96 binding by these 3 candidates in absence of sub-optimal pre-activation was unable to trigger purified T cells proliferation. This observation confirms the co-stimulatory nature of CD96 signaling in human lymphocytes.

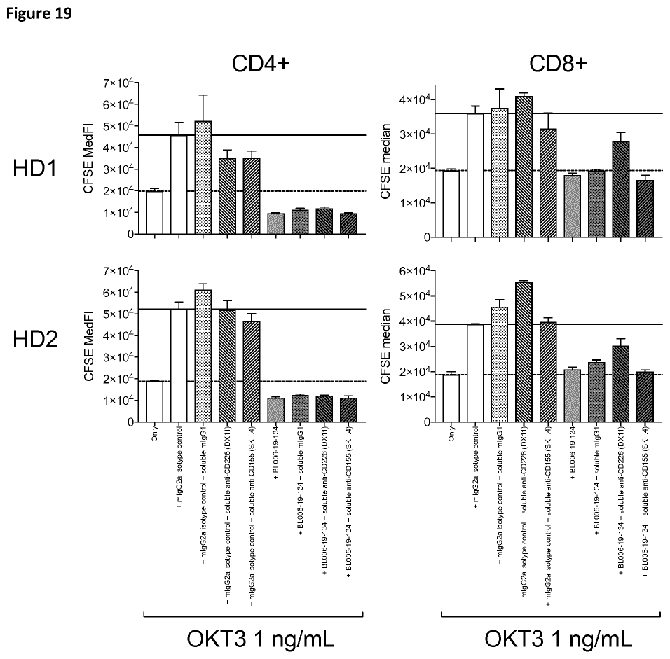

CD96 and CD226 (DNAM-1) are both able to interact with CD155. Engagement of CD226 by CD155 provides a stimulatory signal for NK and T cells. CD155 is expressed on monocytes and activated T cells (data not shown). To rule out the possibility that CD96 masking by candidate antibodies could favor interaction between CD226 and CD155, a co-stimulation experiment of PBMCs was performed in presence of either a blocking antibody to CD226 or a blocking antibody to CD155. Results reported as the median of CFSE fluorescence after 4 days of co-stimulation, indicate that candidate BL006-19-134 was able to induce CD4+ and CD8+ T cells proliferation independently of an interaction between CD226 and CD155 (FIG. 19).

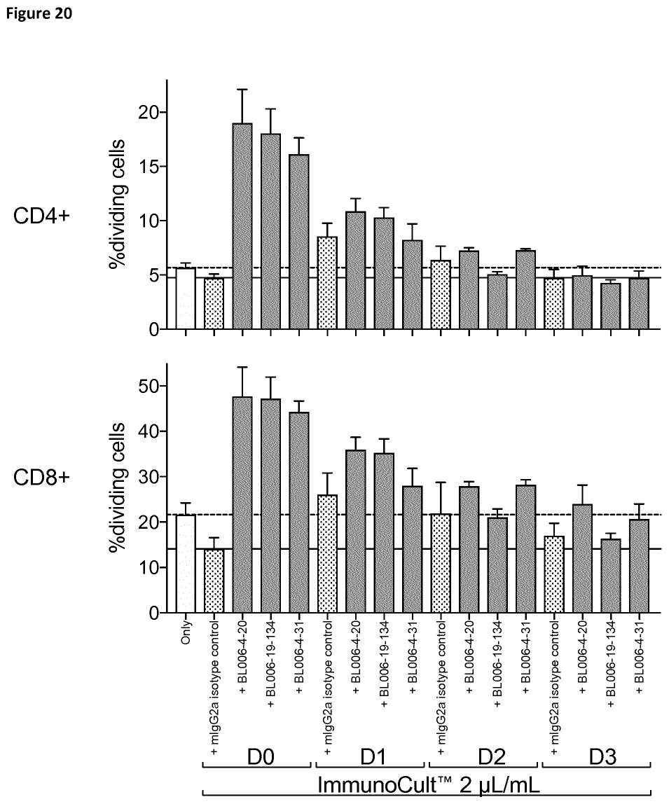

The optimal timing for co-stimulation induced by candidates BL006-4-20, BL006-19-134 and BL006-4-31 was evaluated. Purified CD4+ or CD8+ T cells were sub-optimally activated with 2 μL/mL of tetrameric CD3/CD28 complex (ImmunoCult™, Stemcell). Co-stimulation with anti-CD96 antibodies was either introduced simultaneously or delayed 24 h, 48 h or 72 h. Results indicate that CD96 engagement must be concomitant with sub-optimal CD3 stimulation to obtain the maximal co-stimulation effect (FIG. 20).

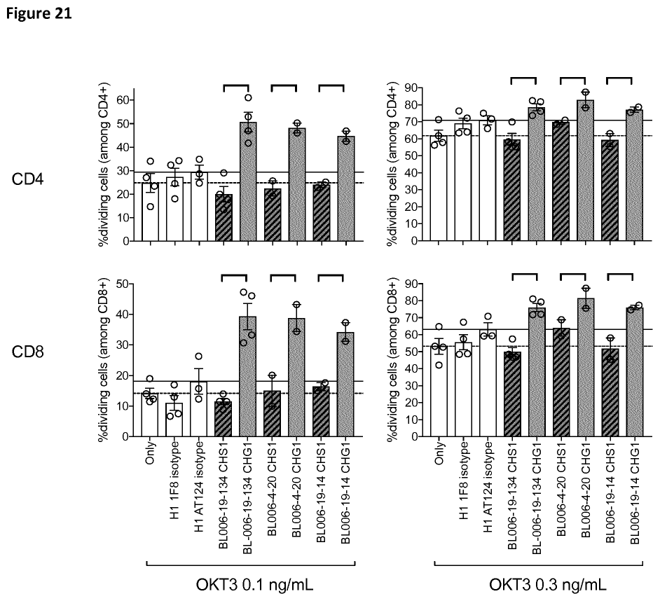

Chimeric antibodies were generated for candidates BL006-4-20, BL006-19-134 and BL006-19-14 on a human IgG1 backbone (CHG1). For each candidate, a Fc silent version (CHS1) was also constructed to evaluate the impact of interaction with Fc receptors on the activity of soluble anti-CD96 candidate antibodies. The effect of these various soluble constructs (1 μg/mL), on proliferation of sub-optimally activated (0.1 and 0.3 ng/mL OKT3) CD4+ and CD8+ T cells was evaluated as previously described. Results indicate that candidates BL006-4-20, BL006-19-134 and BL006-19-14 introduced as soluble human IgG1 in a co-stimulation experiment, are able to co-stimulate CD4+ and CD8+ T cells (FIG. 21). The effect is more marked with the lowest concentration of OKT3. The Fc-silent version of each candidate was unable to induce co-stimulation of CD4+ and CD8+ T cells, indicating that cross-linking by human Fc receptors present on accessory cells in PBMCs was essential to the observed effect.

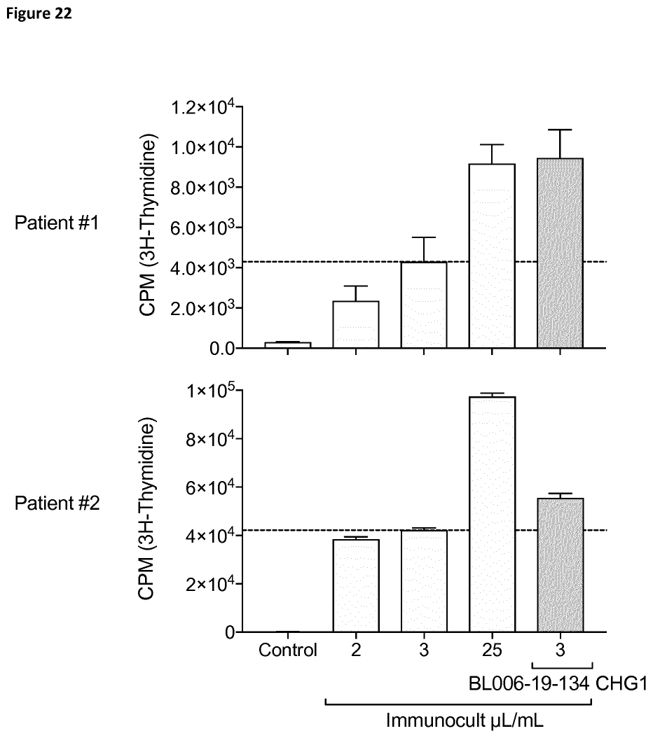

Tumor infiltrating lymphocytes were extracted from surgically removed head and neck cancers from 2 patients who gave their informed consent. This work was approved by the local ethical committee of the hospital of Southampton (UK). The effect of the plate-bound BL006-19-134CHG1 candidate (5 μg/mL) on the proliferation of sub-optimally activated (3 μL/mL Immunocult™) TILs was evaluated after a 5-days co-culture by a 16-hours long pulse of ³[H] Thymidine. Results indicate that candidate BL006-19-134CHG1 introduced as plate-bound human IgG1 in a co-stimulation experiment, is able to co-stimulate and re-invigorate ex vivo TILs from head and neck tumor cells (FIG. 22). The effect was more marked in one of the two experiments where the basal sub-optimal proliferation of TILs was low. In this case, BL006-19-134CHG1-induced co-stimulation induced comparable proliferation to that of an optimal dose of Immunocult™ (25 μL/mL).

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Although the present invention and its advantages have been described in detail, it should be understood that various changes, substitutions and alterations can be made herein without departing from the spirit and scope of the invention as defined in the appended claims.

### REFERENCES

Batard P., Szollosi J., Luescher I., Cerottini J.-C., MacDonald R. and Romero P., Use of phycoerythrin and allophycocyanin for fluorescence resonance energy transfer analyzed by flow cytometry: advantages and limitations, Cytometry, 2002, 48:97-105
Blake S. J., Dougall W. C., Miles J. J., Teng M. W. L, and Smyth M. J., Molecular pathways: Targeting CD96 and TIGIT for cancer immunotherapy, Clinical Cancer Research, 2016, 22(21): 5183-8
Chan C. J., Martinet L., Gilfillan S., Souza-Fonseca-Guimaraes F., Chow M., Town L., Ritchie D. S., Colonna M., Andrews D. M. & Smyth M. J., The receptors CD96 and CD226 oppose each other in the regulation of natural killer cell functions, Nature Immunology, 2014, 15(5):431-8
Chothia C., Novotny J., Bruccoleri R. and Karplus M., Domain association in immunoglobulin molecules. The packing of variable domains, J. Mol. Biol., 1985, 186:651-63
Evan G. I., Lewis G. K., Ramsay G., et al., Isolation of monoclonal antibodies specific for human c-myc proto-oncogene product, Mol. Cell. Biol., 1985, 5(12):3610-6
Fuchs A., Cella M., Giurisato E., Shaw A. S., and Colonna M. Cutting Edge: CD96 (Tactile) Promotes NK Cell-Target Cell Adhesion by Interacting with the Poliovirus Receptor (CD155), Journal of Immunology, 2004, 172:3994-8
Fuhrman C. A., Yeh W.-I., Seay H. R., Saikumar Lakshmi P., Chopra G., Zhang L., Perry D. J., McClymont S. A., Yadav M., Lopez M.-C., Baker H. V., Zhang Y., Li Y., Whitley M., von Schack D., Atkinson M. A., Bluestone J. A. and Brusko T. M., Divergent phenotypes of human regulatory T cells expressing the receptors TIGIT and CD226, The Journal of Immunology, 2015, 195:145-55
Gao J., Chen K., Miao Z., Ren G., Chen X., Gambhir S. S. and Cheng Z., Affibody-based nanoprobes for HER2-expressing cell and tumor imaging, Biomaterials, 2011, 32(8):2141-8
Johnston R. J., Comps-Agrar L., Hackney J., Yu X., Huseni M., Yang Y., Park S., Javinal V., Chiu H., Irving B., Eaton D. L. and Grogan J. L., The immunoreceptor TIGIT regulates antitumor and antiviral CD8+ T cell effector function, Cancer Cell, 2014, 26:923-37
Jokerst J. V., Miao Z., Zavaleta C., Cheng Z., and Gambhir S. S., Affibody-functionalized gold-silica nanoparticles for raman molecular imaging of the epidermal growth factor receptor, Small., 2011, 7(5):625-33
Kennedy J., Vicari A. P., Saylor V., Zurawski, S. M., Copeland N. G., Gilber D. J., Jenkins N. A. and Zlotnik A., A molecular analysis of NKT cells: identification of a class-I restricted T cell-associated molecule (CRTAM), Journal of Leukocyte Biology, 2000, 67:725-34
Novotny J. and Haber E., Structural invariants of antigen binding: comparison of immunoglobulin VL-VH and VL-VL domain dimers., Proc. Natl. Acad. Sci. U.S.A., 1985, 82:4592-96
Lozano E., Dominguez-Villar M., Kuchroo V. and Hafler D. A., The TIGIT/CD226 axis regulates human T cell function, The Journal of Immunology, 2012, 188:3869-75
Melero et al. Therapeutic vaccines for cancer: an overview of clinical trials. Nature reviews. Clinical oncology. 11(9):509-524 (2014)
Paborsky L. R., Fendly B. M., Fisher K. L., et al., Mammalian cell transient expression of tissue factor for the production of antigen, Protein Engineering, 1990, 3(6):547-53
Page D. B., Postow M. A., Callahan M. K., Allison J. P. and Wolchok, J., Immune modulation in cancer with antibodies, Annual Review of Medicine, 2014, 65:185-202
Sanmamed et al. Agonists of co-stimulation in cancer immunotherapy directed against CD137, OX40, GITR, CD27, CD28 and ICOS. Seminars in oncology. 42(4):640-655 (2015)
Saxena A. and Wu, D., Advances in therapeutic Fc engineering—Modulation of IgG-associated effector functions and serum half-life, Frontiers in Immunology, 2016, 12(7):580
Seth S., Maier M. K., Qiu Q., Ravens I., Kremmer E., Förster, R. and Bernhardt G., The murine pan T cell marker CD96 is an adhesion receptor for CD155 and nectin-1, Biochemical and Biophysical Research Communications, 2007, 364:959-65
Shibuya A., Campbell D., Hannum C., Yssel H., Franz-Bacon K., McClanahan T., Kitamura T., Nicholl J., Sutherland G. R., Lanier L. L. and Phillips J. H., DNAM-1, a novel adhesion molecule involved in the cytolytic function of T lymphocytes, Immunity, 1996, 4:573-81
Stanietsky N., Simic H., Arapovic J., Toporik A., Levy O., Novik A., Levine Z., Beiman M., Dassa L., Achdout H., Stern-Ginossar N., Tsukerman P., Jonjic S. and Mandelboim O., The interaction of TIGIT with PVR and PVRL2 inhibits human NK cell cytotoxicity, PNAS, 2009, 106(42): 17858-63
Thakor A. S., Luong R., Paulmurugan R., et al. et al., The fate and toxicity of raman-active silica-gold nanoparticles in mice, Sci. Transl. Med., 2011, 3(79):79ra33
Wang P., O'Farrell S., Clayberger C., Krensky A. Identification and molecular cloning of tactile. A Novel Human T Cell Activation Antigen that Is a Member of the Ig Gene Superfamily, Journal of Immunology, 1992, 148:2600-8
Yu X., Harden K., Gonzales L. C., Francesco M., Chiang E., Irving B., Tom I., Ivejla S., Refino C. J., Clark H., Eaton D. and Grogan J. L., The surface protein TIGIT suppresses T cell activation by promoting the generation of mature immunoregulatory dendritic cells, Nature Immunology, 2009, 10(1):48-57

1.-32. (canceled)

33. A CD96-binding agent, wherein the agent stimulates signaling via CD96, wherein the agent is capable of stimulating activation and/or proliferation of T cells upon binding to CD96, and wherein the agent specifically binds to at least one epitope in an extracellular domain D1 (SEQ ID NO: 269), domain D2 v1 or v2 (SEQ ID NO: 270 or SEQ ID NO: 273), or domain D3 (SEQ ID NO: 271).

34. The CD96-binding agent of claim 33, wherein the CD96-binding agent is capable of stimulating activation and/or proliferation of T cells in combination with a T cell co-stimulatory agent and/or a T cell proliferation agent.

35. The CD96-binding agent of claim 33, wherein binding of the CD96-binding agent to CD96 induces dimerization or multimerization of CD96.

36. The CD96-binding agent of claim 33, wherein the CD96-binding agent is a chimeric, humanized, or fully human anti-CD96 antibody or fragment thereof.

37. The CD96-binding agent of claim 36, wherein the antibody is an F(ab′)2 or a Fab fragment.

38. The CD96-binding agent of claim 36, wherein the antibody comprises the following combinations of 3 heavy light chain CDR sequences with 3 light chain CDR sequences:
a. SEQ ID NOs: 1-3 in combination with SEQ ID NOs: 7-9 (Kabat annotation) or SEQ ID NOs: 4-6 in combination with SEQ ID NOs: 10-12 (IMGT annotation);
b. SEQ ID NOs: 13-15 in combination with SEQ ID NOs: 19-21 (Kabat annotation) or SEQ ID NOs: 16-18 in combination with SEQ ID NOs: 22-24 (IMGT annotation);
c. SEQ ID NOs: 61-63 in combination with SEQ ID NOs: 67-69 (Kabat annotation) or SEQ ID NOs: 64-66 in combination with SEQ ID NOs: 70-72 (IMGT annotation);
d. SEQ ID NOs: 73-75 in combination with SEQ ID NOs: 79-81 (Kabat annotation) or SEQ ID NOs: 76-78 in combination with SEQ ID NOs: 82-84 (IMGT annotation);
e. SEQ ID NOs: 85-87 in combination with SEQ ID NOs: 91-93 (Kabat annotation) or SEQ ID NOs: 88-90 in combination with SEQ ID NOs: 94-96 (IMGT annotation);
f. SEQ ID NOs: 109-111 in combination with SEQ ID NOs: 115-117 (Kabat annotation) or SEQ ID NOs: 112-114 in combination with SEQ ID NOs: 118-120 (IMGT annotation);
g. SEQ ID NOs: 145-147 in combination with SEQ ID NOs: 151-153 (Kabat annotation) or SEQ ID NOs: 148-150 in combination with SEQ ID NOs: 154-156 (IMGT annotation);
h. SEQ ID NOs: 157-159 in combination with SEQ ID NOs: 163-165 (Kabat annotation) or SEQ ID NOs: 160-162 in combination with SEQ ID NOs: 166-168 (IMGT annotation);
i. SEQ ID NOs: 196-171 in combination with SEQ ID NOs: 175-177 (Kabat annotation) or SEQ ID NOs: 172-174 in combination with SEQ ID NOs: 178-180 (IMGT annotation);
j. SEQ ID NOs: 181-183 in combination with SEQ ID NOs: 187-189 (Kabat annotation) or SEQ ID NOs: 184-186 in combination with SEQ ID NOs: 190-192 (IMGT annotation);
k. SEQ ID NOs: 193-195 in combination with SEQ ID NOs: 199-201 (Kabat annotation) or SEQ ID NOs: 196-198 in combination with SEQ ID NOs: 202-204 (IMGT annotation);
l. SEQ ID NOs: 205-207 in combination with SEQ ID NOs: 211-213 (Kabat annotation) or SEQ ID NOs: 208-210 in combination with SEQ ID NOs: 214-216 (IMGT annotation); or
m. SEQ ID NOs: 217-219 in combination with SEQ ID NOs: 223-225 (Kabat annotation) or SEQ ID NOs: 220-222 in combination with SEQ ID NOs: 226-228 (IMGT annotation).

39. The CD96-binding agent of claim 38, wherein the antibody comprises a heavy chain variable domain and a light chain variable domain comprising an amino acid sequence set forth in any one of SEQ ID NOs: 229-244, 247-248, or 253-266.

40. An isolated nucleic acid encoding the amino acid sequence of the CD96-binding agent of claim 38.

41. The CD96-binding agent of claim 33, wherein the T cells are CD4⁺ and/or CD8⁺ cells.

42. The CD96-binding agent of claim 33, wherein the CD96-binding agent binds to human CD96 variant 1 (SEQ ID NO: 267) and/or to human CD96 variant 2 (SEQ ID NO: 268) and/or to rhesus CD96 (SEQ ID NO: 272).

43. The CD96-binding agent of claim 33, wherein the CD96-binding agent is capable of binding to human CD96 expressed on T-cells with a dissociation constant (K_{D}) of less than 50 nM.

44. An isolated cell that produces the CD96-binding agent of claim 33.

45. A pharmaceutical composition comprising the CD96-binding agent of claim 33 and a pharmaceutical acceptable excipient or carrier.

46. A method of treating cancer or an infectious disease, the method comprising administering a therapeutically effective amount of the CD96-binding agent of claim 33 to a patient in need thereof.

47. The method of claim 46, wherein the CD96-binding agent is administered as a monotherapy.

48. The method of claim 46, wherein the CD96-binding agent is administered as a combination therapy.

49. The method of claim 46, wherein the CD96-binding agent is administered in combination with a co-stimulating agent or an immune checkpoint inhibitor.

50. A method of stimulating activation and/or proliferation of T cells upon binding to CD96, the method comprising administering a therapeutically effective amount of the CD96-binding agent of claim 33 to a patient in need thereof.

51. The method of claim 50, wherein the CD96-binding agent is administered at a dosage effective to proliferate T cells when administered in combination with a T cell co-stimulatory agent and/or a T cell proliferation agent.

52. The method of claim 50, wherein the CD96-binding agent is a CD96 agonist.

53. The method of claim 50, wherein binding of the CD96-binding agent to CD96 induces dimerization or multimerization of CD96.

54. The method of claim 50, wherein the T cells are CD4⁺ and/or CD8⁺ cells.

55. A prophylactic or therapeutic vaccine comprising an adjuvant, wherein the adjuvant comprises the CD96-binding agent of claim 33.


SEQUENCE LISTING (274 sequences)

SEQ ID NO: 1 - protein, 5 aa, Artificial Sequence
  heavy chain CDR2-Kabat
SYGMS

SEQ ID NO: 2 - protein, 17 aa, Artificial Sequence
  heavy chain, CDR2,-Kabat
TITHGGSYIYYPDSVKG

SEQ ID NO: 3 - protein, 9 aa, Artificial Sequence
  heavy chain, CDR3-Kabat
HADYARFAY

SEQ ID NO: 4 - protein, 8 aa, Artificial Sequence
  heavy chain, CDR1-IMGT
GFTFSSYG

SEQ ID NO: 5 - protein, 8 aa, Artificial Sequence
  heavy chain, CDR2-IMGT
ITHGGSYI

SEQ ID NO: 6 - protein, 11 aa, Artificial Sequence
  heavy chain, CDR3-IMGT
IRHADYARFAY

SEQ ID NO: 7 - protein, 11 aa, Artificial Sequence
  light chain, CDR1-Kabat
RPSQDISNYLN

SEQ ID NO: 8 - protein, 7 aa, Artificial Sequence
  light chain, CDR2-Kabat
YTSRLHS

SEQ ID NO: 9 - protein, 9 aa, Artificial Sequence
  light chain, CDR3-Kabat
QQGNTLPWT

SEQ ID NO: 10 - protein, 6 aa, Artificial Sequence
  light chain, CDR1-IMGT
QDISNY

SEQ ID NO: 11 - protein, 4 aa, Artificial Sequence
  light chain, CDR2-IMGT
  misc_feature (1)..(1): Xaa can be any naturally occurring amino acid
XYTS

SEQ ID NO: 12 - protein, 9 aa, Artificial Sequence
  light chain, CDR3-IMGT
QQGNTLPWT

SEQ ID NO: 13 - protein, 5 aa, Artificial Sequence
  heavy chain, CDR1-Kabat
SYWMH

SEQ ID NO: 14 - protein, 17 aa, Artificial Sequence
  heavy chain, CDR2-Kabat
QIDPSDSYTNYNQKFKG

SEQ ID NO: 15 - protein, 13 aa, Artificial Sequence
  heavy chain, CDR3-Kabat
GNYGPYLGNYFDY

SEQ ID NO: 16 - protein, 8 aa, Artificial Sequence
  heavy chain, CDR1-IMGT
GYSFTSYW

SEQ ID NO: 17 - protein, 8 aa, Artificial Sequence
  heavy chain, CDR2-IMGT
IDPSDSYT

SEQ ID NO: 18 - protein, 15 aa, Artificial Sequence
  heavy chain, CDR3-IMGT
ASGNYGPYLGNYFDY

SEQ ID NO: 19 - protein, 17 aa, Artificial Sequence
  light chain, CDR1-Kabat
KSSQSLLHSTNQKNYLA

SEQ ID NO: 20 - protein, 7 aa, Artificial Sequence
  light chain, CDR2-Kabat
WASTRES

SEQ ID NO: 21 - protein, 9 aa, Artificial Sequence
  light chain, CDR3-Kabat
QQYYNYPYT

SEQ ID NO: 22 - protein, 12 aa, Artificial Sequence
  light chain, CDR1-IMGT
QSLLHSTNQKNY

SEQ ID NO: 23 - protein, 4 aa, Artificial Sequence
  light chain, CDR2-IMGT
  misc_feature (1)..(1): Xaa can be any naturally occurring amino acid
XWAS

SEQ ID NO: 24 - protein, 9 aa, Artificial Sequence
  light chain, CDR3-IMGT
QQYYNYPYT

SEQ ID NO: 25 - protein, 5 aa, Artificial Sequence
  heavy chain, CDR1-Kabat
NYWMN

SEQ ID NO: 26 - protein, 19 aa, Artificial Sequence
  heavy chain, CDR2-Kabat
QIRFKSDNYATHYAESVKG

SEQ ID NO: 27 - protein, 4 aa, Artificial Sequence
  heavy chain, CDR3-Kabat
  misc_feature (1)..(1): Xaa can be any naturally occurring amino acid
XFAY

SEQ ID NO: 28 - protein, 8 aa, Artificial Sequence
  heavy chain, CDR1-IMGT
GFTFSNYW

SEQ ID NO: 29 - protein, 10 aa, Artificial Sequence
  heavy chain, CDR2-IMGT
IRFKSDNYAT

SEQ ID NO: 30 - protein, 5 aa, Artificial Sequence
  heavy chain, CDR3-IMGT
TEFAY

SEQ ID NO: 31 - protein, 12 aa, Artificial Sequence
  light chain, CDR1-Kabat
SASSRVNSRFLN

SEQ ID NO: 32 - protein, 7 aa, Artificial Sequence
  light chain, CDR2-Kabat
GTSNLAS

SEQ ID NO: 33 - protein, 10 aa, Artificial Sequence
  light chain, CDR3-Kabat
QQYQSDPPIT

SEQ ID NO: 34 - protein, 7 aa, Artificial Sequence
  light chain, CDR1-IMGT
SRVNSRF

SEQ ID NO: 35 - protein, 4 aa, Artificial Sequence
  light chain, CDR2-IMGT
  misc_feature (1)..(1): Xaa can be any naturally occurring amino acid
XGTS

SEQ ID NO: 36 - protein, 10 aa, Artificial Sequence
  light chain, CDR3-IMGT
QQYQSDPPIT

SEQ ID NO: 37 - protein, 5 aa, Artificial Sequence
  heavy chain, CDR1-Kabat
DYEME

SEQ ID NO: 38 - protein, 17 aa, Artificial Sequence
  heavy chain, CDR2-Kabat
AIDPETGGTAYNQKFKG

SEQ ID NO: 39 - protein, 9 aa, Artificial Sequence
  heavy chain, CDR3-Kabat
EGDGSYGAY

SEQ ID NO: 40 - protein, 8 aa, Artificial Sequence
  heavy chain, CDR1-IMGT
GYRFTDYE

SEQ ID NO: 41 - protein, 8 aa, Artificial Sequence
  heavy chain, CDR2-IMGT
IDPETGGT

SEQ ID NO: 42 - protein, 11 aa, Artificial Sequence
  heavy chain, CDR3-IMGT
TREGDGSYGAY

SEQ ID NO: 43 - protein, 16 aa, Artificial Sequence
  light chain, CDR1-Kabat
RSSQSIVYSNGNTYLE

SEQ ID NO: 44 - protein, 7 aa, Artificial Sequence
  light chain, CDR2-Kabat
KVSNRFS

SEQ ID NO: 45 - protein, 9 aa, Artificial Sequence
  light chain, CDR3-Kabat
FQGSHAPWT

SEQ ID NO: 46 - protein, 11 aa, Artificial Sequence
  light chain, CDR1-IMGT
QSIVYSNGNTY

SEQ ID NO: 47 - protein, 4 aa, Artificial Sequence
  light chain, CDR2-IMGT
  misc_feature (1)..(1): Xaa can be any naturally occurring amino acid
XKVS

SEQ ID NO: 48 - protein, 9 aa, Artificial Sequence
  light chain, CDR3-IMGT
FQGSHAPWT

SEQ ID NO: 49 - protein, 5 aa, Artificial Sequence
  heavy chain, CDR1-Kabat
SYAMS

SEQ ID NO: 50 - protein, 17 aa, Artificial Sequence
  heavy chain, CDR2-Kabat
AINTDGASTYFPATVKD

SEQ ID NO: 51 - protein, 10 aa, Artificial Sequence
  heavy chain, CDR3-Kabat
PLEPGSWFAY

SEQ ID NO: 52 - protein, 8 aa, Artificial Sequence
  heavy chain, CDR1-IMGT
GFTFSSYA

SEQ ID NO: 53 - protein, 8 aa, Artificial Sequence
  heavy chain, CDR2-IMGT
INTDGAST

SEQ ID NO: 54 - protein, 12 aa, Artificial Sequence
  heavy chain, CDR3-IMGT
ARPLEPGSWFAY

SEQ ID NO: 55 - protein, 16 aa, Artificial Sequence
  light chain, CDR1-Kabat
RSSQSIVHSNGNTYLE

SEQ ID NO: 56 - protein, 7 aa, Artificial Sequence
  light chain, CDR2-Kabat
KVSNRFS

SEQ ID NO: 57 - protein, 9 aa, Artificial Sequence
  light chain, CDR3-Kabat
FQGSHVPYT

SEQ ID NO: 58 - protein, 11 aa, Artificial Sequence
  light chain, CDR1-IMGT
QSIVHSNGNTY

SEQ ID NO: 59 - protein, 4 aa, Artificial Sequence
  light chain, CDR2-IMGT
  misc_feature (1)..(1): Xaa can be any naturally occurring amino acid
XKVS

SEQ ID NO: 60 - protein, 9 aa, Artificial Sequence
  light chain, CDR3-IMGT
FQGSHVPYT

SEQ ID NO: 61 - protein, 7 aa, Artificial Sequence
  heavy chain, CDR1-Kabat
TFGMGVG

SEQ ID NO: 62 - protein, 16 aa, Artificial Sequence
  heavy chain, CDR2-Kabat
HIWWDDDKFYNPALKS

SEQ ID NO: 63 - protein, 9 aa, Artificial Sequence
  heavy chain, CDR3-Kabat
YYGSLSFDV

SEQ ID NO: 64 - protein, 10 aa, Artificial Sequence
  heavy chain, CDR1-IMGT
GFSLDTFGMG

SEQ ID NO: 65 - protein, 7 aa, Artificial Sequence
  heavy chain, CDR2-IMGT
IWWDDDK

SEQ ID NO: 66 - protein, 11 aa, Artificial Sequence
  heavy chain, CDR3-IMGT
AHYYGSLSFDV

SEQ ID NO: 67 - protein, 16 aa, Artificial Sequence
  light chain, CDR1-Kabat
KSSQSLLDSDGKTYLN

SEQ ID NO: 68 - protein, 7 aa, Artificial Sequence
  light chain, CDR2-Kabat
LVSKLDS

SEQ ID NO: 69 - protein, 9 aa, Artificial Sequence
  light chain, CDR3-Kabat
LQATHFPWT

SEQ ID NO: 70 - protein, 11 aa, Artificial Sequence
  light chain, CDR1-IMGT
QSLLDSDGKTY

SEQ ID NO: 71 - protein, 4 aa, Artificial Sequence
  light chain, CDR2-IMGT
  misc_feature (1)..(1): Xaa can be any naturally occurring amino acid
XLVS

SEQ ID NO: 72 - protein, 9 aa, Artificial Sequence
  light chain, CDR3-IMGT
LQATHFPWT

SEQ ID NO: 73 - protein, 5 aa, Artificial Sequence
  heavy chain, CDR1-Kabat
TYWMH

SEQ ID NO: 74 - protein, 17 aa, Artificial Sequence
  heavy chain, CDR2-Kabat
MIHPNSDTTLYNEKFRS

SEQ ID NO: 75 - protein, 7 aa, Artificial Sequence
  heavy chain, CDR3-Kabat
TGTNFDY

SEQ ID NO: 76 - protein, 8 aa, Artificial Sequence
  heavy chain, CDR1-IMGT
DYTFTTYW

SEQ ID NO: 77 - protein, 8 aa, Artificial Sequence
  heavy chain, CDR2-IMGT
IHPNSDTT

SEQ ID NO: 78 - protein, 9 aa, Artificial Sequence
  heavy chain, CDR3-IMGT
VVTGTNFDY

SEQ ID NO: 79 - protein, 10 aa, Artificial Sequence
  light chain, CDR1-Kabat
SATSSVSYMY

SEQ ID NO: 80 - protein, 7 aa, Artificial Sequence
  light chain, CDR2-Kabat
RTSNLAS

SEQ ID NO: 81 - protein, 9 aa, Artificial Sequence
  light chain, CDR3-Kabat
QQWSSNPLT

SEQ ID NO: 82 - protein, 5 aa, Artificial Sequence
  light chain, CDR1-IMGT
SSVSY

SEQ ID NO: 83 - protein, 4 aa, Artificial Sequence
  light chain, CDR2-IMGT
  misc_feature (1)..(1): Xaa can be any naturally occurring amino acid
XRTS

SEQ ID NO: 84 - protein, 9 aa, Artificial Sequence
  light chain, CDR3-IMGT
QQWSSNPLT

SEQ ID NO: 85 - protein, 7 aa, Artificial Sequence
  heavy chain, CDR1-Kabat
TYGMGVG

SEQ ID NO: 86 - protein, 16 aa, Artificial Sequence
  heavy chain, CDR2-Kabat
NIWWTEDKYYNSALKS

SEQ ID NO: 87 - protein, 11 aa, Artificial Sequence
  heavy chain, CDR3-Kabat
MGPEVYSAMDY

SEQ ID NO: 88 - protein, 10 aa, Artificial Sequence
  heavy chain, CDR1-IMGT
GFSLTTYGMG

SEQ ID NO: 89 - protein, 7 aa, Artificial Sequence
  heavy chain, CDR2-IMGT
IWWTEDK

SEQ ID NO: 90 - protein, 13 aa, Artificial Sequence
  heavy chain, CDR3-IMGT
AQMGPEVYSAMDY

SEQ ID NO: 91 - protein, 11 aa, Artificial Sequence
  light chain, CDR1-Kabat
KASQNVGTAVA

SEQ ID NO: 92 - protein, 7 aa, Artificial Sequence
  light chain, CDR2-Kabat
SASNRNT

SEQ ID NO: 93 - protein, 9 aa, Artificial Sequence
  light chain, CDR3-Kabat
HQYSRYPWT

SEQ ID NO: 94 - protein, 6 aa, Artificial Sequence
  light chain, CDR1-IMGT
QNVGTA

SEQ ID NO: 95 - protein, 4 aa, Artificial Sequence
  light chain, CDR2-IMGT
  misc_feature (1)..(1): Xaa can be any naturally occurring amino acid
XSAS

SEQ ID NO: 96 - protein, 9 aa, Artificial Sequence
  light chain, CDR3-IMGT
HQYSRYPWT

SEQ ID NO: 97 - protein, 5 aa, Artificial Sequence
  heavy chain, CDR1-Kabat
SYWMH

SEQ ID NO: 98 - protein, 17 aa, Artificial Sequence
  heavy chain, CDR2-Kabat
SIYPGSGSTNYDEKFKR

SEQ ID NO: 99 - protein, 4 aa, Artificial Sequence
  heavy chain, CDR3-Kabat
EMAY

SEQ ID NO: 100 - protein, 8 aa, Artificial Sequence
  heavy chain, CDR1-IMGT
GYKFTSYW

SEQ ID NO: 101 - protein, 8 aa, Artificial Sequence
  heavy chain, CDR2-IMGT
IYPGSGST

SEQ ID NO: 102 - protein, 6 aa, Artificial Sequence
  heavy chain, CDR3-IMGT
TREMAY

SEQ ID NO: 103 - protein, 16 aa, Artificial Sequence
  light chain, CDR1-Kabat
RSSKSLLYKDGKTYLN

SEQ ID NO: 104 - protein, 7 aa, Artificial Sequence
  light chain, CDR2-Kabat
WMSTRAS

SEQ ID NO: 105 - protein, 9 aa, Artificial Sequence
  light chain, CDR3-Kabat
QQVVEYPFT

SEQ ID NO: 106 - protein, 11 aa, Artificial Sequence
  light chain, CDR1-IMGT
KSLLYKDGKTY

SEQ ID NO: 107 - protein, 4 aa, Artificial Sequence
  light chain, CDR2-IMGT
  misc_feature (1)..(1): Xaa can be any naturally occurring amino acid
XWMS

SEQ ID NO: 108 - protein, 9 aa, Artificial Sequence
  light chain, CDR3-IMGT
QQVVEYPFT

SEQ ID NO: 109 - protein, 5 aa, Artificial Sequence
  heavy chain, CDR1-Kabat
DYYIN

SEQ ID NO: 110 - protein, 17 aa, Artificial Sequence
  heavy chain, CDR2-Kabat
WIFPGSGSSFYSEIFKG

SEQ ID NO: 111 - protein, 13 aa, Artificial Sequence
  heavy chain, CDR3-Kabat
RNYYGYGAYAMDY

SEQ ID NO: 112 - protein, 8 aa, Artificial Sequence
  heavy chain, CDR1-IMGT
GFTFTDYY

SEQ ID NO: 113 - protein, 8 aa, Artificial Sequence
  heavy chain, CDR2-IMGT
IFPGSGSS

SEQ ID NO: 114 - protein, 15 aa, Artificial Sequence
  heavy chain, CDR3-IMGT
ARRNYYGYGAYAMDY

SEQ ID NO: 115 - protein, 11 aa, Artificial Sequence
  light chain, CDR1-Kabat
RASQSINFWLS

SEQ ID NO: 116 - protein, 7 aa, Artificial Sequence
  light chain, CDR2-Kabat
KASNLHT

SEQ ID NO: 117 - protein, 9 aa, Artificial Sequence
  light chain, CDR3-Kabat
LQGQSYPLT

SEQ ID NO: 118 - protein, 6 aa, Artificial Sequence
  light chain, CDR1-IMGT
QSINFW

SEQ ID NO: 119 - protein, 4 aa, Artificial Sequence
  light chain, CDR2-IMGT
  misc_feature (1)..(1): Xaa can be any naturally occurring amino acid
XYTS

SEQ ID NO: 120 - protein, 9 aa, Artificial Sequence
  light chain, CDR3-IMGT
LQGQSYPLT

SEQ ID NO: 121 - protein, 5 aa, Artificial Sequence
  heavy chain, CDR1-Kabat
SYAMS

SEQ ID NO: 122 - protein, 17 aa, Artificial Sequence
  heavy chain, CDR2-Kabat
AIQSNGGSTFYPDTVKD

SEQ ID NO: 123 - protein, 5 aa, Artificial Sequence
  heavy chain, CDR3-Kabat
GPFDY

SEQ ID NO: 124 - protein, 8 aa, Artificial Sequence
  heavy chain, CDR1-IMGT
GFTFSSYA

SEQ ID NO: 125 - protein, 8 aa, Artificial Sequence
  heavy chain, CDR2-IMGT
IQSNGGST

SEQ ID NO: 126 - protein, 7 aa, Artificial Sequence
  heavy chain, CDR3-IMGT
VRGPFDY

SEQ ID NO: 127 - protein, 10 aa, Artificial Sequence
  light chain, CDR1-Kabat
SASSSVNYIH

SEQ ID NO: 128 - protein, 7 aa, Artificial Sequence
  light chain, CDR2-Kabat
DTSKLTP

SEQ ID NO: 129 - protein, 9 aa, Artificial Sequence
  light chain, CDR3-Kabat
HQWRSYPPT

SEQ ID NO: 130 - protein, 5 aa, Artificial Sequence
  light chain, CDR1-IMGT
SSVNY

SEQ ID NO: 131 - protein, 4 aa, Artificial Sequence
  light chain, CDR2-IMGT
  misc_feature (1)..(1): Xaa can be any naturally occurring amino acid
XDTS

SEQ ID NO: 132 - protein, 9 aa, Artificial Sequence
  light chain, CDR3-IMGT
HQWRSYPPT

SEQ ID NO: 133 - protein, 5 aa, Artificial Sequence
  heavy chain, CDR1-Kabat
SYNMH

SEQ ID NO: 134 - protein, 17 aa, Artificial Sequence
  heavy chain, CDR2-Kabat
AVIPGNGDSFYNQKFKG

SEQ ID NO: 135 - protein, 8 aa, Artificial Sequence
  heavy chain, CDR3-Kabat
SSSGCIAY

SEQ ID NO: 136 - protein, 8 aa, Artificial Sequence
  heavy chain, CDR1-IMGT
GYTFTSYN

SEQ ID NO: 137 - protein, 8 aa, Artificial Sequence
  heavy chain, CDR2-IMGT
VIPGNGDS

SEQ ID NO: 138 - protein, 9 aa, Artificial Sequence
  heavy chain, CDR3-IMGT
RSSSGCIAY

SEQ ID NO: 139 - protein, 15 aa, Artificial Sequence
  light chain, CDR1-Kabat
RVSGSVDYDGDSYMN

SEQ ID NO: 140 - protein, 7 aa, Artificial Sequence
  light chain, CDR2-Kabat
AASNLES

SEQ ID NO: 141 - protein, 9 aa, Artificial Sequence
  light chain, CDR3-Kabat
LQSNEAPWT

SEQ ID NO: 142 - protein, 10 aa, Artificial Sequence
  light chain, CDR1-IMGT
GSVDYDGDSY

SEQ ID NO: 143 - protein, 4 aa, Artificial Sequence
  light chain, CDR2-IMGT
  misc_feature (1)..(1): Xaa can be any naturally occurring amino acid
XAAS

SEQ ID NO: 144 - protein, 9 aa, Artificial Sequence
  light chain, CDR3-IMGT
LQSNEAPWT

SEQ ID NO: 145 - protein, 5 aa, Artificial Sequence
  heavy chain, CDR1-Kabat
SYWIT

SEQ ID NO: 146 - protein, 17 aa, Artificial Sequence
  heavy chain, CDR2-Kabat
DIYPGSGHTNYNEKFKS

SEQ ID NO: 147 - protein, 8 aa, Artificial Sequence
  heavy chain, CDR3-Kabat
PTVEAMDY

SEQ ID NO: 148 - protein, 8 aa, Artificial Sequence
  heavy chain, CDR1-IMGT
GYTFISYW

SEQ ID NO: 149 - protein, 8 aa, Artificial Sequence
  heavy chain, CDR2-IMGT
IYPGSGHT

SEQ ID NO: 150 - protein, 10 aa, Artificial Sequence
  heavy chain, CDR3-IMGT
ARPTVEAMDY

SEQ ID NO: 151 - protein, 10 aa, Artificial Sequence
  light chain, CDR1-Kabat
SASSSVNYMH

SEQ ID NO: 152 - protein, 7 aa, Artificial Sequence
  light chain, CDR2-Kabat
DTSKLAS

SEQ ID NO: 153 - protein, 9 aa, Artificial Sequence
  light chain, CDR3-Kabat
QQWSNNPLT

SEQ ID NO: 154 - protein, 5 aa, Artificial Sequence
  light chain, CDR1-IMGT
SSVNY

SEQ ID NO: 155 - protein, 4 aa, Artificial Sequence
  light chain, CDR2-IMGT
  misc_feature (1)..(1): Xaa can be any naturally occurring amino acid
XDTS

SEQ ID NO: 156 - protein, 9 aa, Artificial Sequence
  light chain, CDR3-IMGT
QQWSNNPLT

SEQ ID NO: 157 - protein, 7 aa, Artificial Sequence
  heavy chain, CDR1-Kabat
TFGMGVS

SEQ ID NO: 158 - protein, 16 aa, Artificial Sequence
  heavy chain, CDR2-Kabat
HIFWDDDKRYNPSLKS

SEQ ID NO: 159 - protein, 7 aa, Artificial Sequence
  heavy chain, CDR3-Kabat
GSNWFAY

SEQ ID NO: 160 - protein, 10 aa, Artificial Sequence
  heavy chain, CDR1-IMGT
GFSLSTFGMG

SEQ ID NO: 161 - protein, 7 aa, Artificial Sequence
  heavy chain, CDR2-IMGT
IFWDDDK

SEQ ID NO: 162 - protein, 9 aa, Artificial Sequence
  heavy chain, CDR3-IMGT
TRGSNWFAY

SEQ ID NO: 163 - protein, 15 aa, Artificial Sequence
  light chain, CDR1-Kabat
RASESVDSYGNSFMH

SEQ ID NO: 164 - protein, 7 aa, Artificial Sequence
  light chain, CDR2-Kabat
RASNLES

SEQ ID NO: 165 - protein, 9 aa, Artificial Sequence
  light chain, CDR3-Kabat
QQSNEDPLT

SEQ ID NO: 166 - protein, 10 aa, Artificial Sequence
  light chain, CDR1-IMGT
ESVDSYGNSF

SEQ ID NO: 167 - protein, 4 aa, Artificial Sequence
  light chain, CDR2-IMGT
  misc_feature (1)..(1): Xaa can be any naturally occurring amino acid
XRAS

SEQ ID NO: 168 - protein, 9 aa, Artificial Sequence
  light chain, CDR3-IMGT
QQSNEDPLT

SEQ ID NO: 169 - protein, 5 aa, Artificial Sequence
  heavy chain, CDR1-Kabat
DNWID

SEQ ID NO: 170 - protein, 17 aa, Artificial Sequence
  heavy chain, CDR2-Kabat
NIFPGGNYTNYNEKFKG

SEQ ID NO: 171 - protein, 9 aa, Artificial Sequence
  heavy chain, CDR3-Kabat
AVADSQFAY

SEQ ID NO: 172 - protein, 8 aa, Artificial Sequence
  heavy chain, CDR1-IMGT
GYTFTDNW

SEQ ID NO: 173 - protein, 8 aa, Artificial Sequence
  heavy chain, CDR2-IMGT
IFPGGNYT

SEQ ID NO: 174 - protein, 11 aa, Artificial Sequence
  heavy chain, CDR3-IMGT
VRAVADSQFAY

SEQ ID NO: 175 - protein, 10 aa, Artificial Sequence
  light chain, CDR1-Kabat
SASSRVTYMY

SEQ ID NO: 176 - protein, 7 aa, Artificial Sequence
  light chain, CDR2-Kabat
RTSNLAS

SEQ ID NO: 177 - protein, 9 aa, Artificial Sequence
  light chain, CDR3-Kabat
QQWSSNPLT

SEQ ID NO: 178 - protein, 5 aa, Artificial Sequence
  light chain, CDR1-IMGT
SRVTY

SEQ ID NO: 179 - protein, 4 aa, Artificial Sequence
  light chain, CDR2-IMGT
  misc_feature (1)..(1): Xaa can be any naturally occurring amino acid
XRTS

SEQ ID NO: 180 - protein, 9 aa, Artificial Sequence
  light chain, CDR3-IMGT
QQWSSNPLT

SEQ ID NO: 181 - protein, 5 aa, Artificial Sequence
  heavy chain, CDR1-Kabat
NYGMS

SEQ ID NO: 182 - protein, 17 aa, Artificial Sequence
  heavy chain, CDR2-Kabat
TINSNGGSTYYPDSVKG

SEQ ID NO: 183 - protein, 8 aa, Artificial Sequence
  heavy chain, CDR3-Kabat
RGAWYFDV

SEQ ID NO: 184 - protein, 8 aa, Artificial Sequence
  heavy chain, CDR1-IMGT
GFTFSNYG

SEQ ID NO: 185 - protein, 8 aa, Artificial Sequence
  heavy chain, CDR2-IMGT
INSNGGST

SEQ ID NO: 186 - protein, 9 aa, Artificial Sequence
  heavy chain, CDR3-IMGT
KRGAWYFDV

SEQ ID NO: 187 - protein, 11 aa, Artificial Sequence
  light chain, CDR1-Kabat
KASEDIYNRLA

SEQ ID NO: 188 - protein, 7 aa, Artificial Sequence
  light chain, CDR2-Kabat
SATSLET

SEQ ID NO: 189 - protein, 10 aa, Artificial Sequence
  light chain, CDR3-Kabat
QQYWTTPTWT

SEQ ID NO: 190 - protein, 6 aa, Artificial Sequence
  light chain, CDR1-IMGT
EDIYNR

SEQ ID NO: 191 - protein, 4 aa, Artificial Sequence
  light chain, CDR2-IMGT
  misc_feature (1)..(1): Xaa can be any naturally occurring amino acid
XSAT

SEQ ID NO: 192 - protein, 10 aa, Artificial Sequence
  light chain, CDR3-IMGT
QQYWTTPTWT

SEQ ID NO: 193 - protein, 5 aa, Artificial Sequence
  heavy chain, CDR1-Kabat
SYWMH

SEQ ID NO: 194 - protein, 17 aa, Artificial Sequence
  heavy chain, CDR2-Kabat
MIHPNGDITDYNEKFKS

SEQ ID NO: 195 - protein, 8 aa, Artificial Sequence
  heavy chain, CDR3-Kabat
ASYYGSLY

SEQ ID NO: 196 - protein, 8 aa, Artificial Sequence
  heavy chain, CDR1-IMGT
GYTFTSYW

SEQ ID NO: 197 - protein, 8 aa, Artificial Sequence
  heavy chain, CDR2-IMGT
IHPNGDIT

SEQ ID NO: 198 - protein, 10 aa, Artificial Sequence
  heavy chain, CDR3-IMGT
ARASYYGSLY

SEQ ID NO: 199 - protein, 11 aa, Artificial Sequence
  light chain, CDR1-Kabat
KASQNVGIAVA

SEQ ID NO: 200 - protein, 7 aa, Artificial Sequence
  light chain, CDR2-Kabat
SASTRYT

SEQ ID NO: 201 - protein, 6 aa, Artificial Sequence
  light chain, CDR3-Kabat
QNVGIA

SEQ ID NO: 202 - protein, 6 aa, Artificial Sequence
  light chain, CDR1-IMGT
QNVGIA

SEQ ID NO: 203 - protein, 4 aa, Artificial Sequence
  light chain, CDR2-IMGT
  misc_feature (1)..(1): Xaa can be any naturally occurring amino acid
XSAS

SEQ ID NO: 204 - protein, 9 aa, Artificial Sequence
  light chain, CDR3-IMGT
QQYGSSPLT

SEQ ID NO: 205 - protein, 5 aa, Artificial Sequence
  heavy chain, CDR1-Kabat
TYWIH

SEQ ID NO: 206 - protein, 17 aa, Artificial Sequence
  heavy chain, CDR2-Kabat
MILPNSGTTIYNEKFRS

SEQ ID NO: 207 - protein, 7 aa, Artificial Sequence
  heavy chain, CDR3-Kabat
TGTNFDY

SEQ ID NO: 208 - protein, 8 aa, Artificial Sequence
  heavy chain, CDR1-IMGT
GYTFTTYW

SEQ ID NO: 209 - protein, 8 aa, Artificial Sequence
  heavy chain, CDR2-IMGT
ILPNSGTT

SEQ ID NO: 210 - protein, 9 aa, Artificial Sequence
  heavy chain, CDR3-IMGT
AVTGTNFDY

SEQ ID NO: 211 - protein, 10 aa, Artificial Sequence
  light chain, CDR1-Kabat
SASSSVSYMY

SEQ ID NO: 212 - protein, 7 aa, Artificial Sequence
  light chain, CDR2-Kabat
RTSNLAS

SEQ ID NO: 213 - protein, 9 aa, Artificial Sequence
  light chain, CDR3-Kabat
QQWSSNPLT

SEQ ID NO: 214 - protein, 5 aa, Artificial Sequence
  light chain, CDR1-IMGT
SSVSY

SEQ ID NO: 215 - protein, 4 aa, Artificial Sequence
  light chain, CDR2-IMGT
  misc_feature (1)..(1): Xaa can be any naturally occurring amino acid
XRTS

SEQ ID NO: 216 - protein, 9 aa, Artificial Sequence
  light chain, CDR3-IMGT
QQWSSNPLT

SEQ ID NO: 217 - protein, 5 aa, Artificial Sequence
  heavy chain, CDR1-Kabat
NYWIG

SEQ ID NO: 218 - protein, 17 aa, Artificial Sequence
  heavy chain, CDR2-Kabat
DIYPGGGYTNYNEKFKG

SEQ ID NO: 219 - protein, 13 aa, Artificial Sequence
  heavy chain, CDR3-Kabat
SGYYDGSPLAMDY

SEQ ID NO: 220 - protein, 8 aa, Artificial Sequence
  heavy chain, CDR1-IMGT
GYTFTNYW

SEQ ID NO: 221 - protein, 8 aa, Artificial Sequence
  heavy chain, CDR2-IMGT
IYPGGGYT

SEQ ID NO: 222 - protein, 15 aa, Artificial Sequence
  heavy chain, CDR3-IMGT
ARSGYYDGSPLAMDY

SEQ ID NO: 223 - protein, 10 aa, Artificial Sequence
  light chain, CDR1-Kabat
SASSSVSYMH

SEQ ID NO: 224 - protein, 7 aa, Artificial Sequence
  light chain, CDR2-Kabat
DTSKLAS

SEQ ID NO: 225 - protein, 9 aa, Artificial Sequence
  light chain, CDR3-Kabat
QQWSSNPPT

SEQ ID NO: 226 - protein, 5 aa, Artificial Sequence
  light chain, CDR1-IMGT
SSVSY

SEQ ID NO: 227 - protein, 4 aa, Artificial Sequence
  light chain, CDR2-IMGT
  misc_feature (1)..(1): Xaa can be any naturally occurring amino acid
XDTS

SEQ ID NO: 228 - protein, 9 aa, Artificial Sequence
  light chain, CDR3-IMGT
QQWSSNPPT

SEQ ID NO: 229 - protein, 118 aa, Artificial Sequence
  variable region heavy chain
EVQLAESGGDLVTPGGSLKLSCAASGFTFSSYGMSWVRQTPDKRLEWVATITHGGSYIYYPDSVKGRFTLSRDNAKNTLYLQMSSLKSEDTAMYYCIRHADYARFAYWGQGTLVTVSA

SEQ ID NO: 230 - protein, 107 aa, Artificial Sequence
  variable region light chain
DIQMTQTTSSLSASLGDRVTISCRPSQDISNYLNWYQQKPDGTVKLLIYYTSRLHSGVPSRFSGSGSGTDYSLTISNLEQEDIATYFCQQGNTLPWTFGGGTKLEIK

SEQ ID NO: 231 - protein, 122 aa, Artificial Sequence
  variable region heavy chain
QVQLQQPGAELVKPGASVKLSCKASGYSFTSYWMHWVKQRPGQGLEWIGQIDPSDSYTNYNQKFKGKATLTVDKSSSTAYMQLSSLTSEDSAVYYCASGNYGPYLGNYFDYWGQGTTLTVPS

SEQ ID NO: 232 - protein, 113 aa, Artificial Sequence
  variable region light chain
DIVMSQSPSSLVVSVGEKVTMSCKSSQSLLHSTNQKNYLAWYQQKPGQSPKLLIYWASTRESGVPDRFTGSGSGTDFTLTISSVKAEDLVVYYCQQYYNYPYTFGGGTKLEIK

SEQ ID NO: 233 - protein, 114 aa, Artificial Sequence
  variable region heavy chain
EVKLEESGGGLVQPGGSMKLSCVASGFTFSNYWMNWVRQSPEKGLEWVAQIRFKSDNYATHYAESVKGRFTISRDDSKSSVYLQMHNLRAEDTGIYYCTEFAYWGQGTLVTVSA

SEQ ID NO: 234 - protein, 109 aa, Artificial Sequence
  variable region light chain
QILLTQSPAIMSASPGEKVTMTCSASSRVNSRFLNWYQQKSGVSPKLWIYGTSNLASGVPARFSGSGSGTSYSLTISSVEAEDVATYYCQQYQSDPPITFGAGTRLELI

SEQ ID NO: 235 - protein, 118 aa, Artificial Sequence
  variable region heavy chain
QVQLQQSGAELVRPGASVKLSCKASGYRFTDYEMEWVKQTPVHGLEWIGAIDPETGGTAYNQKFKGKATVTADKSSRTAYMELRSLTSEDSAVYYCTREGDGSYGAYWGQGTLVTVSA

SEQ ID NO: 236 - protein, 112 aa, Artificial Sequence
  variable region light chain
DVMMTQTPLSLPVSLGDQASISCRSSQSIVYSNGNTYLEWYLQKPGQSPKLLMYKVSNRFSGVPDRFSGSGSGTDFTLKISRVEAEDLGVYYCFQGSHAPWTFGGGTKLEIK

SEQ ID NO: 237 - protein, 119 aa, Artificial Sequence
  variable region heavy chain
EVQLVESGGGLVKPGGSLKLSCAASGFTFSSYAMSWVRQTPEKRLEWVAAINTDGASTYFPATVKDRFTISRDNAKNTLYLQMSSLRSEDTALYYCARPLEPGSWFAYWGQGTLVTVSA

SEQ ID NO: 238 - protein, 112 aa, Artificial Sequence
  variable region light chain
DVLMTQTPLSLPVSLGDQASISCRSSQSIVHSNGNTYLEWYLQKPGQSPKVLIYKVSNRFSGVPDRISGSGSGTDFTLKISRVEAGDLGVYYCFQGSHVPYTFGGGTKLEIK

SEQ ID NO: 239 - protein, 119 aa, Artificial Sequence
  variable region heavy chain
QVTLKESGPGILQPSQTLSLTCSFSGFSLDTFGMGVGWIRQSSGKGLEWLAHIWWDDDKFYNPALKSRLTVSKDTSKNQVFLKIANVDTADTATYYCAHYYGSLSFDVWGTGTTVTVSS

SEQ ID NO: 240 - protein, 112 aa, Artificial Sequence
  variable region light chain
DVVMTQTPLTLSVTIGQPASISCKSSQSLLDSDGKTYLNWLLQRPGESPKLLIYLVSKLDSGVPDRFTGSGSGTDFTLKISRVEAEDLGVYYCLQATHFPWTFGGGTKLEIK

SEQ ID NO: 241 - protein, 116 aa, Artificial Sequence
  variable region heavy chain
QVQLQQPGAELVKPGASVKLSCKASDYTFTTYWMHWVKQRPGQGLEWIGMIHPNSDTTLYNEKFRSQATLTVDKSSSTAYIQLSRLTSEDSAVYYCVVTGTNFDYWGQGTTLAVSS

SEQ ID NO: 242 - protein, 106 aa, Artificial Sequence
  variable region light chain
QVVLTQSPAIMSASPGEKVTMTCSATSSVSYMYWYQQKPGSSPKPWIYRTSNLASGVPVRFSGSGSGTSYSLTISSMEAEDAATYYCQQWSSNPLTFGGGTKLELK

SEQ ID NO: 243 - protein, 121 aa, Artificial Sequence
  variable region heavy chain
QVTLNESGPGILQPSQTLSLTCSFSGFSLTTYGMGVGWIRQPSGKSLEWLANIWWTEDKYYNSALKSRLTISTDTSHNQVFLKISSVDTEDTATYYCAQMGPEVYSAMDYWGQGTSVTVSS

SEQ ID NO: 244 - protein, 107 aa, Artificial Sequence
  variable region light chain
DIVMTPSQKFMSTTVGDRVSITCKASQNVGTAVAWYQQKPGHSPKLLIYSASNRNTGVPDRFTGSGSGTDFTLTISNMQSEDLADYFCHQYSRYPWTFGGGTKLEIK

SEQ ID NO: 245 - protein, 113 aa, Artificial Sequence
  variable region heavy chain
QVQLQQPGSELVRPGASVKLSCKASGYKFTSYWMHWVKQRPGQGLEWIGSIYPGSGSTNYDEKFKRKATLTVDTFSSTASMQLSSLTSEDSAVYYCTREMAYWGQGTLVTVSA

SEQ ID NO: 246 - protein, 112 aa, Artificial Sequence
  variable region light chain
DIVITQDELSNPVTSGESVSISCRSSKSLLYKDGKTYLNWFLQRPGQSPQLLVYWMSTRASGVSDRFSGSGSGTDFTLEISRVKAEDVGVYYCQQVVEYPFTFGTGTKLGIK

SEQ ID NO: 247 - protein, 122 aa, Artificial Sequence
  variable region heavy chain
QVQLQQSGPELVKPGASVKISCKTSGFTFTDYYINWVKQRPGQGLEWIGWIFPGSGSSFYSEIFKGQATLTVDKSSSTAYMLLSSLTSEDSAVYFCARRNYYGYGAYAMDYWGQGTSVTVSS

SEQ ID NO: 248 - protein, 107 aa, Artificial Sequence
  variable region light chain
DIQMNQSPSSLSASLGDTITITCRASQSINFWLSWYQQKPGNIPKLLIYKASNLHTGVPSRFSGSGSGTDFTLTISSLQPEDIATYYCLQGQSYPLTFGGGTKLEIK

SEQ ID NO: 249 - protein, 114 aa, Artificial Sequence
  variable region heavy chain
EVKLVESGGGLVKPGGSLKLSCAASGFTFSSYAMSWVRQTPERGLEWVAAIQSNGGSTFYPDTVKDRFTISRDNAKNTLYLQMSSLRSEDTALYYCVRGPFDYWGQGTTLTVSS

SEQ ID NO: 250 - protein, 106 aa, Artificial Sequence
  variable region light chain
QIVLTQSPAIMSASPGQKVTIPCSASSSVNYIHWFQQKLGSSPKLWIYDTSKLTPGVPARFSGSGSGTSYSLTISSMEAEDAASYFCHQWRSYPPTFGGGTKLEIK

SEQ ID NO: 251 - protein, 117 aa, Artificial Sequence
  variable region heavy chain
QAYLQQSGAELVRPGASVKMSCKASGYTFTSYNMHWVKQTPRQGLEWIGAVIPGNGDSFYNQKFKGKATLTVDKSSSTAYMQLSSLTSEDSAVYFCGRSSSGCIAYWGQGTLVTVSA

SEQ ID NO: 252 - protein, 111 aa, Artificial Sequence
  variable region light chain
DIVLTQSPASLAVSLGQRATISCRVSGSVDYDGDSYMNWYQQKPGQPPKLLIYAASNLESGIPARFSGSGSETDFTLNIHPVEEEDAATYYCLQSNEAPWTFGGGTKVEIK

SEQ ID NO: 253 - protein, 117 aa, Artificial Sequence
  variable region heavy chain
QVQLQQPGAELVKPGASVKMSCKASGYTFISYWITWVKQRPGQGLEWIGDIYPGSGHTNYNEKFKSKATLTVDTSSSTAYMQLSSLTSEDSAVYYCARPTVEAMDYWGQGTSVTVSS

SEQ ID NO: 254 - protein, 106 aa, Artificial Sequence
  variable region light chain
QIVLTQSPAIMSASPGEKVTMTCSASSSVNYMHWYQQRSGTSPKRWIYDTSKLASGVPARFSGSGSGTSYSLTISSMEAEDAATYYCQQWSNNPLTFGAGTKLELK

SEQ ID NO: 255 - protein, 117 aa, Artificial Sequence
  variable region heavy chain
QVTLKESGPGILQSSQTLSLTCSFSGFSLSTFGMGVSWIRKPSGKGLEWLAHIFWDDDKRYNPSLKSRLTISKDTSSNQVFLMINSMDIADTATYYCTRGSNWFAYWGQGTLVTVSA

SEQ ID NO: 256 - protein, 111 aa, Artificial Sequence
  variable region light chain
DIVLTQSPASLAVSLGQRATISCRASESVDSYGNSFMHWYQQKPGQPPKLLIYRASNLESGIPARFSGSGSRTDFTLTINPVEADDVATYYCQQSNEDPLTFGAGTKLELK

SEQ ID NO: 257 - protein, 118 aa, Artificial Sequence
  variable region heavy chain
QVQLQQSGAEVVRPGTSVKMSCKAAGYTFTDNWIDWVKQRPGHGLDWIGNIFPGGNYTNYNEKFKGKATLTADTSSSTAYMQLINLTSEDSAIYYCVRAVADSQFAYWGQGTLVTVSA

SEQ ID NO: 258 - protein, 106 aa, Artificial Sequence
  variable region light chain
QIVLTQSPAIMSVSPGEKVTMTCSASSRVTYMYWYQQKPGSSPKPWIYRTSNLASGVPVRFSGSGSGTSYSLTISSMEAEDAATYYCQQWSSNPLTFGAGTKLELK

SEQ ID NO: 259 - protein, 117 aa, Artificial Sequence
  variable region heavy chain
EVQLVESGGGLVQPGGSLKLSCAASGFTFSNYGMSWVRQTPDKRLELVATINSNGGSTYYPDSVKGRFTISRDNAKNTLYLQMSSLKSEDTAMYYCTKRGAWYFDVWGTGTTVTVSS

SEQ ID NO: 260 - protein, 108 aa, Artificial Sequence
  variable region light chain
DIQMTQSSSSFSVSLGDRVTITCKASEDIYNRLAWYQQKPGNAPRLLISSATSLETGVSSRFSGSGSGKDYTLSITSLQTEDVATYYCQQYWTTPTWTFGGGTKLEIK

SEQ ID NO: 261 - protein, 117 aa, Artificial Sequence
  variable region heavy chain
QVQLQQPGAELVKPGASVKLSCKASGYTFTSYWMHWVKQRPGQGLEWIGMIHPNGDITDYNEKFKSKATLAVDESSSTAFMQLNSLTSEDSAFYYCARASYYGSLYWGQGTTLTVSS

SEQ ID NO: 262 - protein, 107 aa, Artificial Sequence
  variable region light chain
DIVMTQSQKFMSTSVGDRVSVTCKASQNVGIAVAWYQKKPGQSPKTLIYSASTRYTGVPDRFTGSGSGTDFTLTINDVQSEDLADYFCQQYGSSPLTFGAGTKLELK

SEQ ID NO: 263 - protein, 116 aa, Artificial Sequence
  variable region heavy chain
QVQLQQPGAELVKPGASVKLSCKASGYTFTTYWIHWVKQRPGQGLEWIGMILPNSGTTIYNEKFRSQATLTVDKSSSAAYMQLSRLTSEDSAVYYCAVTGTNFDYWGQGTTLTVSS

SEQ ID NO: 264 - protein, 106 aa, Artificial Sequence
  variable region light chain
QIVLTQSPAIMSASPGEKVTMTCSASSSVSYMYWYQQKPGSSPKPWIYRTSNLASGVPVRFSGSGFGTSYSLTISNMEAEDAATYYCQQWSSNPLTFGTGTKLELK

SEQ ID NO: 265 - protein, 121 aa, Artificial Sequence
  variable region heavy chain
QVQLQQSGAELGRPGTSVKMSCKAAGYTFTNYWIGWVKQRPGHGLEWIGDIYPGGGYTNYNEKFKGKATLTADTSSSTAYIQLSSLTSEDSAIYYCARSGYYDGSPLAMDYWGQGTSVTVS

SEQ ID NO: 266 - protein, 106 aa, Artificial Sequence
  variable region light chain
QIVLTQSPTIMSASPGEKVTMTCSASSSVSYMHWYQQKSGTSPKRWIYDTSKLASGVPARFSGSGSGTSYSLTISSMEAEDAATYYCQQWSSNPPTFGSGTKLEIK

SEQ ID NO: 267 - protein, 585 aa, Artificial Sequence
  Human CD96, variant 1 (long isoform)
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQYQKEIMERPPPFKPPPPPIKYTCIQEPNESDLPYHEMETL

SEQ ID NO: 268 - protein, 569 aa, Artificial Sequence
  Human CD96, variant 2 (long isoform)
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQYQKEIMERPPPFKPPPPPIKYTCIQEPNESDLPYHEMETL

SEQ ID NO: 269 - protein, 88 aa, Artificial Sequence
  extracellular domain D1 of human CD96, variant 1 (long isoform)
GSDVNLTCQTQTVGFFVQMQWSKVTNKIDLIAVYHPQYGFYCAYGRPCESLVTFTETPENGSKWTLHLRNMSCSVSGRYECMLVLYPE

SEQ ID NO: 270 - protein, 83 aa, Artificial Sequence
  extracellular domain D2 of human CD96, variant 1 (long isoform)
NQTLEIPCFQNSSSKISSEFTYAWSVENSSTDSWVLLSKGIKEDNGTQETLISQNHLISNSTLLKDRVKLGTDYRLHLSPVQI

SEQ ID NO: 271 - protein, 107 aa, Artificial Sequence
  extracellular domain D3 of human CD96, variant 1 (long isoform)
PEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKIT

SEQ ID NO: 272 - protein, 569 aa, Artificial Sequence
  rhesus CD96, from NCBI accession number XP_ 00548247
MEKKWKYCAVYYIIQIHFVKGVWGKPLNTEENIYATLGSDVNLTCQTQAKGFLVQMQWSKVTDKADLIALYHPQYGFHCAYGSPCESLVTFTQTPENGSKWTLHLRNMSSSVSGRYECMLTLYPEGMQTKIYNLLIQTHVTPDEWKSNHTIEIEINQTLEIPCFQNSSSEISSEFTYAWLVEDNGTQQTLISQDHLISSSTLLKDRVKVGTDYRLHLSPVQIFDDGRKFSCHIRVGPDKILRSSTTIKVFAKPEIPMIVENNSTDVLVERTFTCLLTNVFPKANIIWFIDGSFLHDEKEGIYITNEERKGKDGFLELKSVLTRVHSDKPAQSDNLTIWCMALSPVPGNKVWNISSEKITFLLGSEMSTTDPPPSVTESTLDTQPSPASSVSPTRYPATSSVTLADVSALRPNTTPQSSSSSVTTQDFNYPWTSSGTDAKKSFSQIPSETYSLSPSGAGSTLHDNVFTSTTRALSEVPTTANGSTKTNHVHITGIVVSKPKDGMSWPVIVAALLFCCMILFVLGVRKWCQYQKEIMERPPPFKPPPPPIKYTCIQEPNESDPPYHEMETL

SEQ ID NO: 273 - protein, 67 aa, Artificial Sequence
  extracellular domain D2 of human CD96, variant 2 (long isoform)
NQTLEIPCFQNSSSKISSEFTYAWSVEDNGTQETLISQNHLISNSTLLKDRVKLGTDYRLHLSPVQI

SEQ ID NO: 274 - protein, 133 aa, Artificial Sequence
  extracellular domain D4 of human CD96, variant 1 (long isoform)
SSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKT